(19) United States
(12) Patent Application Publication
(10) Pub. No.: US 2025/0366310 A1
(43) Pub. Date: Nov. 27, 2025

(54) ORGANIC COMPOUND AND APPLICATION THEREOF

(71) Applicants: Wuhan Tianma Microelectronics Co., Ltd., Wuhan (CN); Wuhan Tianma Microelectronics Co., Ltd. Shanghai Branch, Shanghai (CN)

(72) Inventors: Wei GAO, Wuhan (CN); Wenpeng DAI, Wuhan (CN)

(73) Assignees: Wuhan Tianma Microelectronics Co., Ltd., Wuhan (CN); Wuhan Tianma Microelectronics Co., Ltd. Shanghai Branch, Shanghai (CN)

(21) Appl. No.: 19/043,160

(22) Filed: Jan. 31, 2025

(30) Foreign Application Priority Data
Jun. 27, 2024 (CN) 202410850407.7

(51) Int. Cl. H10K 50/844; C07D 209/08; C07D 251/22; C07D 311/80; C09K 11/06

(52) U.S. Cl. CPC H10K 50/844; C07D 209/08; C07D 251/22; C07D 311/80; C09K 11/06; C09K 2211/1007; C09K 2211/1029

(57) ABSTRACT

Provided are an organic compound and an application thereof. The organic compound has a structure represented by Formula I. Through a design of a molecular structure, the organic compound has a low refractive index, no absorption within a visible light wavelength range, a low extinction coefficient within a wavelength range of 400-600 nm and an excellent light extraction effect, thereby significantly improving the luminescence efficiency of an element as a material of a capping layer. Moreover, the organic compound has a relatively high glass transition temperature and excellent thermal stability, thereby meeting a processing requirement of evaporation. In addition, a small difference is between refractive indexes of the organic compound in different light colors, and when display is performed at multiple angles, the color cast can be effectively improved, thereby giving a more excellent light emission and display effect to the element.

### CROSS-REFERENCE TO RELATED APPLICATIONS

This application claims priority to Chinese Patent Application No. 202410850407.7, filed on Jun. 27, 2024, the contents of which are incorporated herein in its entirety by reference.

### TECHNICAL FIELD

The present disclosure belongs to the technical field of organic electroluminescence and specifically relates to an organic compound and an application thereof.

### BACKGROUND

An organic light-emitting diode (OLED), which has advantages such as an ultra-thin thickness, self-luminescence, a wide viewing angle, flexibility, a fast response, high efficiency, good temperature adaptability, a simple process and a low drive voltage, has made great progress in recent years and has been widely used in industries such as flat-panel display, flexible display, solid-state lighting and in-vehicle display. After decades of development, although the internal quantum efficiency of the OLED element is close to 100%, the external quantum efficiency is only about 20%. Most light is confined inside a light-emitting element due to factors such as a loss of a substrate mode, a surface plasmon loss and a waveguide effect, resulting in a loss of a large amount of energy.

To improve the luminescence efficiency of the OLED element, in a top emitting element, an organic capping layer (CPL) is deposited through evaporation on a translucent metal electrode for adjusting an optical interference distance, suppressing the reflection of external light and suppressing the extinction caused by the movement of surface plasmon, thereby improving light extraction efficiency and luminescence efficiency.

To meet performance requirements of the element, a CPL material usually needs to meet the following requirements: no absorption in a visible light wavelength region (400 to 700 nm), a high refractive index (generally, the refractive index n>2.1) or a low refractive index (generally, the refractive index n is 1.5 to 1.7), a low extinction coefficient (k≤0.00) within a wavelength range of 400-600 nm, a high glass transition temperature and molecular thermal stability and evaporation without the occurrence of thermal decomposition. However, many problems still exist in a CPL material in the related art, for example, a requirement for a refractive index cannot be met, an improvement effect on luminescence efficiency is not apparent, and apparent color cast exists in an element, thereby affecting a display effect. Therefore, how to develop more types of CPL materials with higher performance to improve the luminescence efficiency and color cast of the OLED element is an urgent problem to be solved in the art.

### SUMMARY

To improve the efficiency of an OLED element, improve the color cast and develop more types of CPL materials with higher performance, a first object of the present disclosure is to provide an organic compound. The organic compound has a structure represented by Formula I:

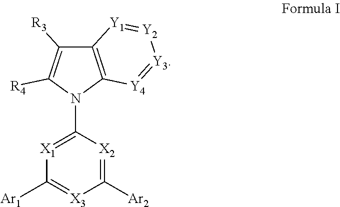

In Formula I, Y₁, Y₂, Y₃ and Y₄ are each independently selected from CR₁ or N.

In Formula I, X₁, X₂ and X₃ are each independently selected from CR₂ or N.

R₁, R₂, R₃ and R₄ are each independently selected from any one of hydrogen, deuterium, halogen, cyano, isocyano, substituted or unsubstituted C1 to C20 linear or branched alkyl, substituted or unsubstituted C1 to C20 alkoxy, substituted or unsubstituted C1 to C20 alkylthio, substituted or unsubstituted C3 to C20 cycloalkyl, substituted or unsubstituted C6 to C30 aryl or substituted or unsubstituted C6 to C30 arylsilyl.

In Formula I, Ar₁ and Ar₂ are each independently selected from any one of halogen, cyano, isocyano, substituted or unsubstituted C1 to C20 linear or branched alkyl, substituted or unsubstituted C1 to C20 alkoxy, substituted or unsubstituted C1 to C20 alkylthio, substituted or unsubstituted C3 to C20 cycloalkyl, substituted or unsubstituted C6 to C30 aryl, substituted or unsubstituted C3 to C30 heteroaryl, substituted or unsubstituted C6 to C30 ketoaryl, substituted or unsubstituted C3 to C30 ketoheteroaryl, substituted or unsubstituted C6 to C30 aryl sulfonyl, substituted or unsubstituted C3 to C30 heteroaryl sulfonyl, substituted or unsubstituted C6 to C30 aryl phosphorus oxygen or substituted or unsubstituted C3 to C30 heteroaryl phosphorus oxygen.

Substituents for substitutions in R₁, R₂, R₃, R₄, Ar₁ and Ar₂ are each independently selected from at least one of deuterium, halogen, cyano, isocyano, unsubstituted or halogenated C1 to C12 linear or branched alkyl, C3 to C12 cycloalkyl, unsubstituted or halogenated C1 to C12 alkoxy, unsubstituted or halogenated C1 to C12 alkylthio, C6 to C20 aryl, C3 to C20 heteroaryl, C6 to C20 aryl ester or C6 to C20 arylsilyl.

The organic compound provided in the present disclosure has the structure represented by Formula I. Through a design of a molecular structure, the organic compound has a low refractive index, no absorption within a visible light wavelength (400 to 700 nm) range, a low extinction coefficient within a wavelength range of 400-600 nm, an excellent light extraction effect, a relatively high glass transition temperature and excellent thermal stability. Moreover, an extreme small difference is between refractive indexes of the organic compound in different light colors, and when display is performed at multiple angles, the color cast can be effectively improved, thereby improving the luminescence efficiency of an element.

A second object of the present disclosure is to provide a light extraction material including the organic compound as described in the first object.

A third object of the present disclosure is to provide an organic electroluminescent element including an anode, a cathode and an organic layer disposed between the anode and the cathode, where a first capping layer is further disposed on a side of the cathode facing away from the anode, and the first capping layer includes the organic compound as described in the first object.

A fourth object of the present disclosure is to provide another organic electroluminescent element including an anode, a cathode and an organic layer disposed between the anode and the cathode, where the organic layer includes at least one organic compound as described in the first object.

A fifth object of the present disclosure is to provide a display device including the organic electroluminescent element as described in the third object or the fourth object.

Compared with the related art, the present disclosure has the beneficial effects described below.

The organic compound provided in the present disclosure has the molecular structure represented by Formula I. Through the design of the molecular structure, the organic compound has the low refractive index, no absorption within the visible light wavelength range, the low extinction coefficient within the wavelength range of 400-600 nm and the excellent light extraction effect, thereby significantly improving the luminescence efficiency of the element as a material of the capping layer. Moreover, the organic compound has the relatively high glass transition temperature and the excellent thermal stability, thereby meeting a processing requirement of evaporation. In addition, the small difference is between the refractive indexes of the organic compound in the different light colors, and when the display is performed at the multiple angles, the color cast can be effectively improved, thereby giving a more excellent light emission and display effect to the element.

### BRIEF DESCRIPTION OF DRAWINGS

FIG. 1 is a structure diagram of an organic electroluminescent element according to an embodiment of the present disclosure.

FIG. 2 is a structure diagram of an organic electroluminescent element according to another embodiment of the present disclosure.

### REFERENCE LIST

110 anode
120 hole transport region
121 hole injection layer
122 first hole transport layer
123 second hole transport layer
130 light-emitting layer
140 electron transport region
141 first electron transport layer
142 second electron transport layer
150 cathode
160 second capping layer
170 first capping layer
180 substrate

### DETAILED DESCRIPTION

Technical solutions of the present disclosure are further described below through specific examples. It is to be understood by those skilled in the art that the examples described below are used for a better understanding of the present disclosure and are not to be construed as specific limitations to the present disclosure.

In the present disclosure, a feature defined as a “first” feature or a “second” feature may explicitly or implicitly include one or more of such features to distinguish and describe features regardless of order or weight. In the description of the present disclosure, unless otherwise noted, the phrase of “a plurality of” means two or more.

In the present disclosure, it is found through studies that in an OLED element, since a requirement for a refractive index of an existing CPL material cannot be met, the CPL does not have an apparent improvement effect on the luminescence efficiency of the element. The luminescence efficiency of the element is still insufficient, and the problem of apparent color cast exists, thereby affecting a display effect.

To improve the efficiency of the OLED element, improve the color cast and develop more types of CPL materials with higher performance, an embodiment of the present disclosure provides an organic compound. The organic compound has a structure represented by Formula I:

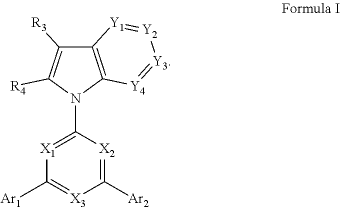

In Formula I, Y₁, Y₂, Y₃ and Y₄ are each independently selected from CR₁ or N.

In Formula I, X₁, X₂ and X₃ are each independently selected from CR₂ or N.

R₁, R₂, R₃ and R₄ are each independently selected from any one of hydrogen, deuterium, halogen, cyano, isocyano, substituted or unsubstituted C1 to C20 linear or branched alkyl, substituted or unsubstituted C1 to C20 alkoxy, substituted or unsubstituted C1 to C20 alkylthio, substituted or unsubstituted C3 to C20 cycloalkyl, substituted or unsubstituted C6 to C30 aryl or substituted or unsubstituted C6 to C30 arylsilyl.

In Formula I, Ar₁ and Ar₂ are each independently selected from any one of halogen, cyano, isocyano, substituted or unsubstituted C1 to C20 linear or branched alkyl, substituted or unsubstituted C1 to C20 alkoxy, substituted or unsubstituted C1 to C20 alkylthio, substituted or unsubstituted C3 to C20 cycloalkyl, substituted or unsubstituted C6 to C30 aryl, substituted or unsubstituted C3 to C30 heteroaryl, substituted or unsubstituted C6 to C30 ketoaryl, substituted or unsubstituted C3 to C30 ketoheteroaryl, substituted or unsubstituted C6 to C30 aryl sulfonyl, substituted or unsubstituted C3 to C30 heteroaryl sulfonyl, substituted or unsubstituted C6 to C30 aryl phosphorus oxygen or substituted or unsubstituted C3 to C30 heteroaryl phosphorus oxygen.

In the present disclosure, the organic compound contains an indole/azaindole structure. An N atom is joined to a six-membered aromatic ring where X₁ is located by a single bond, and meta-substituted Ar₁ and Ar₂ are further joined to the six-membered aromatic ring. In this manner, a molecular framework of the compound is formed. Through a design of the molecular structure, the organic compound has a relatively low refractive index (the refractive index n is 1.5 to 1.7), no absorption within a visible light wavelength range (400 to 700 nm) and a low extinction coefficient (k≤0.00) within a wavelength range of 400-600 nm and can achieve an excellent light extraction effect. Moreover, the organic compound has a relatively high glass transition temperature and good molecular thermal stability, thereby meeting a processing requirement of evaporation without the occurrence of thermal decomposition. In particular, an extremely small difference is between refractive indexes of the organic compound in different light colors, and when display is performed at multiple angles, the color cast can be effectively improved, thereby significantly improving the luminescence efficiency of the element and giving more excellent light emission and display performance to the element.

As a material of a capping layer of the organic electroluminescent element (OLED), the organic compound provided in the present disclosure is particularly suitable for matching with a CPL material (for example, n>1.9) with a high refractive index so that the element has higher luminescence efficiency and lower color cast, thereby improving accuracy of color display.

In the present disclosure, the halogen may be F, Cl, Br or I.

In the present disclosure, the C1 to C20 linear or branched alkyl may be, for example, C1, C2, C3, C4, C5, C6, C7, C8, C9, C10, C11, C12, C13, C14, C15, C16, C17, C18 or C20 linear or branched alkyl, preferably C1 to C12 linear or branched alkyl. The C1 to C20 linear or branched alkyl exemplarily includes, but is not limited to, methyl, ethyl, n-propyl, isopropyl, n-butyl, isobutyl, tert-butyl, n-pentyl, isopentyl, neopentyl, n-hexyl, n-octyl, n-heptyl, n-nonyl, n-decyl or the like.

In the present disclosure, the C1 to C20 alkoxy may be, for example, C1, C2, C3, C4, C5, C6, C7, C8, C9, C10, C11, C12, C13, C14, C15, C16, C17, C18 or C20 linear or branched alkoxy, preferably C1 to C12 alkoxy. A specific example of the C1 to C20 alkoxy is a monovalent group formed after O is joined to the linear or branched alkyl listed above.

In the present disclosure, the C1 to C20 alkylthio may be, for example, C1, C2, C3, C4, C5, C6, C7, C8, C9, C10, C11, C12, C13, C14, C15, C16, C17, C18 or C20 linear or branched alkylthio, preferably C1 to C12 alkylthio. A specific example of the C1 to C20 alkylthio is a monovalent group formed after S is joined to the linear or branched alkyl listed above.

In the present disclosure, the C3 to C20 cycloalkyl may be, for example, C3, C4, C5, C6, C7, C8, C9, C10, C11, C12, C13, C14, C15, C16, C17, C18 or C20 cycloalkyl, including monocycloalkyl or polycycloalkyl. The C3 to C20 cycloalkyl exemplarily includes, but is not limited to, cyclopropyl, cyclobutyl, cyclopentyl, cyclohexyl, adamantyl or the like.

In the present disclosure, the C6 to C30 aryl may be, for example, C6, C8, C9, C10, C12, C13, C14, C15, C16, C18, C20, C22, C24, C26 or C28 monocyclic aryl or fused-ring aryl. The C6 to C30 aryl exemplarily includes, but is not limited to, phenyl, biphenyl, terphenyl, naphthyl, anthryl, phenanthryl, indenyl, fluorenyl and derivatives thereof (9,9-dimethylfluorenyl, 9,9-diethylfluorenyl, 9,9-diphenylfluorenyl, 9,9-dinaphthylfluorenyl, spirobifluorenyl, benzofluorenyl or the like), fluoranthenyl, triphenylene, pyrenyl, perylenyl, chrysenyl, naphthacenyl or the like. It is to be noted that a compound formed after monocyclic aryl and fused-ring aryl are joined by a single bond also falls within a range of aryl, for example, phenylnaphthyl, naphthylphenyl or binaphthyl.

In the present disclosure, the C3 to C30 heteroaryl may be, for example, C3, C4, C5, C6, C8, C9, C10, C12, C13, C14, C15, C16, C18, C20, C22, C24, C26 or C28 monocyclic heteroaryl or fused-ring heteroaryl, where a heteroatom in the heteroaryl includes O, S, N, P, B or the like. The C3 to C30 heteroaryl exemplarily includes, but is not limited to, furanyl, thienyl, pyrrolyl, pyridyl, pyrazinyl, pyridazinyl, pyrimidinyl, triazinyl, quinolinyl, isoquinolinyl, quinazolinyl, quinoxalinyl, cinnolinyl, ortho-phenanthrolinyl, imidazolyl, thiazolyl, oxazolyl, benzimidazolyl, benzothiazolyl, benzoxazolyl, benzofuranyl, benzothienyl, indolyl, dibenzofuranyl, dibenzothienyl, carbazolyl and derivatives thereof (N-phenylcarbazolyl, N-naphthylcarbazolyl, benzocarbazolyl, dibenzocarbazolyl, indolocarbazolyl, azacarbazolyl or the like), phenothiazinyl, phenoxazinyl, hydroacridinyl or the like. The heteroaryl further includes a monovalent group formed after the heteroaryl and aryl listed above are joined by a single bond.

In the present disclosure, the C6 to C30 arylsilyl may be, for example, C6, C8, C9, C10, C12, C13, C14, C15, C16, C18, C20, C22, C24, C26 or C28 arylsilyl, preferably C6 to C20 arylsilyl. A specific example of the C6 to C30 arylsilyl is a monovalent group formed after at least one H in —SiH₃ is substituted with the aryl listed above.

In the present disclosure, the C6 to C30 ketoaryl may be, for example, C6, C8, C9, C10, C12, C13, C14, C15, C16, C18, C20, C22, C24, C26 or C28 ketoaryl. A specific example of the C6 to C30 ketoaryl is a monovalent group formed after the aryl listed above is joined to a ketone group

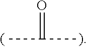

The C3 to C30 ketoheteroaryl may be, for example, C3, C4, C5, C6, C8, C9, C10, C12, C13, C14, C15, C16, C18, C20, C22, C24, C26 or C28 ketoheteroaryl. A specific example of the C3 to C30 ketoheteroaryl is a monovalent group formed after the heteroaryl listed above is joined to a ketone group

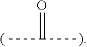

In the present disclosure, the C6 to C30 aryl sulfonyl may be, for example, C6, C8, C9, C10, C12, C13, C14, C15, C16, C18, C20, C22, C24, C26 or C28 aryl sulfonyl. A specific example of the C6 to C30 aryl sulfonyl is a monovalent group formed after the aryl listed above is joined to a sulfonyl group

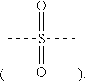

The C3 to C30 heteroaryl sulfonyl may be, for example, C3, C4, C5, C6, C8, C9, C10, C12, C13, C14, C15, C16, C18, C20, C22, C24, C26 or C28 heteroaryl sulfonyl. A specific example of the C3 to C30 heteroaryl sulfonyl is a monovalent group formed after the heteroaryl listed above is joined to a sulfonyl group

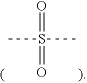

In the present disclosure, the substituted or unsubstituted C6 to C30 aryl phosphorus oxygen may be, for example, C6, C8, C9, C10, C12, C13, C14, C15, C16, C18, C20, C22, C24, C26 or C28 aryl phosphorus oxygen. A specific example of the substituted or unsubstituted C6 to C30 aryl phosphorus oxygen is a monovalent group formed after the aryl listed above is joined to a phosphorus oxygen group

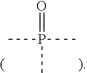

The C3 to C30 heteroaryl phosphorus oxygen may be, for example, C3, C4, C5, C6, C8, C9, C10, C12, C13, C14, C15, C16, C18, C20, C22, C24, C26 or C28 heteroaryl phosphorus oxygen. A specific example of the C3 to C30 heteroaryl phosphorus oxygen is a monovalent group formed after the heteroaryl listed above is joined to a phosphorus oxygen group

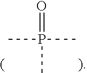

In an embodiment, substituents for substitutions in R₁, R₂, R₃, R₄, Ar₁ and Ar₂ are each independently selected from at least one of deuterium, halogen, cyano, isocyano, unsubstituted or halogenated C1 to C12 (for example, C1, C2, C3, C4, C5, C6, C7, C8, C9, C10, C11 or C12) linear or branched alkyl, C3 to C12 (for example, C3, C4, C5, C6, C7, C8, C9, C10, C11 or C12) cycloalkyl, unsubstituted or halogenated C1 to C12 (for example, C1, C2, C3, C4, C5, C6, C7, C8, C9, C10, C11 or C12) alkoxy, unsubstituted or halogenated C1 to C12 (for example, C1, C2, C3, C4, C5, C6, C7, C8, C9, C10, C11 or C12) alkylthio, C6 to C20 (for example, C6, C8, C9, C10, C12, C13, C14, C15, C16, C18 or the like) aryl, C3 to C20 (for example, C3, C4, C5, C6, C8, C9, C10, C12, C13, C14, C15, C16, C18 or the like) heteroaryl, C6 to C20 (for example, C6, C8, C9, C10, C12, C13, C14, C15, C16, C18 or the like) aryl ester or C6 to C20 (for example, C6, C8, C9, C10, C12, C13, C14, C15, C16, C18 or the like) arylsilyl.

In the present disclosure, the “substituted or unsubstituted” group may include one substituent or multiple substituents. In the presence of multiple (at least two) substituents, the multiple (at least two) substituents are the same group or different groups; where the substituent may be joined at any position of the group where the substituent can be joined. The same expressions involved below all have the same meanings. If no special description is provided, the selection range of the substituents for the substitutions is shown above and is not repeated.

In an embodiment, Y₁, Y₂, Y₃ and Y₄ are each independently selected from CR₁ or N, that is, the number of N in Y₁, Y₂, Y₃ and Y₄ is 0, 1, 2 or 3.

In some optional embodiments of the present disclosure, Y₁, Y₂, Y₃ and Y₄ are each independently selected from CR₁ or N, and the number of N≤2.

In some optional embodiments of the present disclosure, Y₁, Y₂, Y₃ and Y₄ are each independently selected from CR₁ or N, and the number of N is 0 or 1.

In some optional embodiments of the present disclosure, the organic compound has a structure represented by any one of Formula II-1, Formula II-2, Formula II-3, Formula II-4 or Formula II-5:

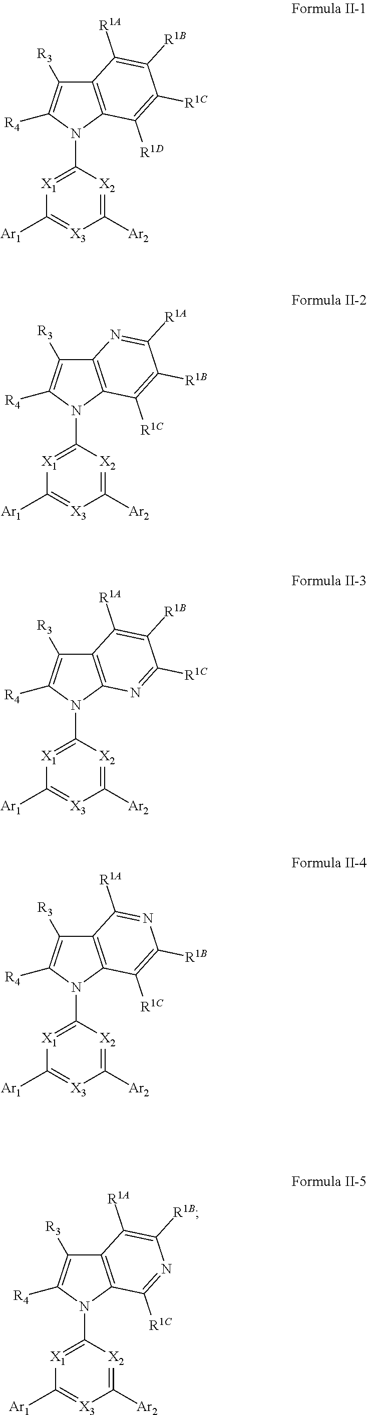

where X₁, X₂, X₃, R₃, R₄, Ar₁ and Ar₂ have the same defined range as Formula I.

R^{1A}, R^{1B}, R^{1C} and R^{1D} are each independently selected from any one of hydrogen, deuterium, halogen, cyano, isocyano, substituted or unsubstituted C1 to C20 linear or branched alkyl, substituted or unsubstituted C1 to C20 alkoxy, substituted or unsubstituted C1 to C20 alkylthio, substituted or unsubstituted C3 to C20 cycloalkyl, substituted or unsubstituted C6 to C30 aryl, substituted or unsubstituted C3 to C30 heteroaryl or substituted or unsubstituted C6 to C30 arylsilyl.

Substituents for substitutions in RIA, R^{1B}, R^{1C} and R^{1D} are each independently selected from at least one of deuterium, halogen, cyano, isocyano, unsubstituted or halogenated C1 to C12 linear or branched alkyl, C3 to C12 cycloalkyl, unsubstituted or halogenated C1 to C12 alkoxy, unsubstituted or halogenated C1 to C12 alkylthio, C6 to C20 aryl, C3 to C20 heteroaryl, C6 to C20 aryl ester or C6 to C20 arylsilyl.

In an embodiment, X₁, X₂ and X₃ are each independently selected from CR₂ or N, that is, the number of N in X₁, X₂ and X₃ is 0, 1, 2 or 3.

In some optional embodiments of the present disclosure, X₁, X₂ and X₃ are each independently selected from CH or N, and the number of N is 0, 1, 2 or 3.

In some optional embodiments of the present disclosure, R₁, R₃ and R₄ are each independently selected from any one of hydrogen, deuterium, halogen, cyano, unsubstituted or halogenated C1 to C12 (for example, C1, C2, C3, C4, C5, C6, C7, C8, C9, C10, C11 or C12) linear or branched alkyl, C3 to C12 (for example, C3, C4, C5, C6, C7, C8, C9, C10, C11 or C12) cycloalkyl, unsubstituted or halogenated C1 to C12 (for example, C1, C2, C3, C4, C5, C6, C7, C8, C9, C10, C11 or C12) alkoxy or unsubstituted or halogenated C1 to C12 (for example, C1, C2, C3, C4, C5, C6, C7, C8, C9, C10, C11 or C12) alkylthio.

In some optional embodiments of the present disclosure, R₁, R₃ and R₄ are each independently selected from any one of hydrogen, deuterium, halogen, cyano, unsubstituted or halogenated C1 to C6 linear or branched alkyl, C3 to C10 cycloalkyl, unsubstituted or halogenated C1 to C6 alkoxy or unsubstituted or halogenated C1 to C6 alkylthio.

In some optional embodiments of the present disclosure, R₁, R₃ and R₄ are each independently selected from any one of hydrogen, deuterium, halogen, cyano, methyl, trifluoromethyl, adamantyl, methoxy or trifluoromethoxy.

In some optional embodiments of the present disclosure, R^{1A}, R^{1B}, R^{1C}, R^{1D}, R₃ and R₄ are each independently selected from any one of hydrogen, deuterium, halogen, cyano, unsubstituted or halogenated C1 to C12 (for example, C1, C2, C3, C4, C5, C6, C7, C8, C9, C10, C11 or C12) linear or branched alkyl, C3 to C12 (for example, C3, C4, C5, C6, C7, C8, C9, C10, C11 or C12) cycloalkyl, unsubstituted or halogenated C1 to C12 (for example, C1, C2, C3, C4, C5, C6, C7, C8, C9, C10, C11 or C12) alkoxy or unsubstituted or halogenated C1 to C12 (for example, C1, C2, C3, C4, C5, C6, C7, C8, C9, C10, C11 or C12) alkylthio.

In some optional embodiments of the present disclosure, R^{1A}, R^{1B}, R^{1C}, R^{1D}, R₃ and R₄ are each independently selected from any one of hydrogen, deuterium, halogen, cyano, unsubstituted or halogenated C1 to C6 linear or branched alkyl, C3 to C10 cycloalkyl, unsubstituted or halogenated C1 to C6 alkoxy or unsubstituted or halogenated C1 to C6 alkylthio.

In some optional embodiments of the present disclosure, R^{1A}, R^{1B}, R^{1C}, R^{1D}, R₃ and R₄ are each independently selected from any one of hydrogen, deuterium, halogen, cyano, methyl, trifluoromethyl, adamantyl, methoxy or trifluoromethoxy.

In some optional embodiments of the present disclosure, in Formula II-1, at least two (for example, two, three or four) of RIA, R^{1B}, R^{1C} and R^{1D} are hydrogen.

In some optional embodiments of the present disclosure, in Formula II-2, Formula II-3, Formula II-4 or Formula II-5, at least one (for example, one, two or three) of RIA, R^{1B} or R^{1C} is hydrogen.

In some optional embodiments of the present disclosure, Ar₁ and Ar₂ are each independently selected from any one of substituted or unsubstituted C6 to C30 aryl, substituted or unsubstituted C3 to C30 heteroaryl, substituted or unsubstituted C6 to C30 ketoaryl, substituted or unsubstituted C3 to C30 ketoheteroaryl, substituted or unsubstituted C6 to C30 aryl sulfonyl or substituted or unsubstituted C6 to C30 aryl phosphorus oxygen.

In some optional embodiments of the present disclosure, Ar₁ and Ar₂ are each independently selected from any one of substituted or unsubstituted C6 to C20 (for example, C6, C9, C10, C12, C14, C15, C16, C18 or the like) aryl, substituted or unsubstituted C3 to C20 (for example, C3, C4, C5, C6, C9, C10, C12, C14, C15, C16, C18 or the like) heteroaryl, substituted or unsubstituted C6 to C20 (for example, C6, C9, C10, C12, C14, C15, C16, C18 or the like) ketoaryl, substituted or unsubstituted C3 to C20 (for example, C3, C4, C5, C6, C9, C10, C12, C14, C15, C16, C18 or the like) ketoheteroaryl, substituted or unsubstituted C6 to C20 (for example, C6, C9, C10, C12, C14, C15, C16, C18 or the like) aryl sulfonyl or substituted or unsubstituted C6 to C20 (for example, C6, C9, C10, C12, C14, C15, C16, C18 or the like) aryl phosphorus oxygen.

In some optional embodiments of the present disclosure, substituents for substitutions in Ar₁ and Ar₂ are each independently selected from at least one of deuterium, halogen, cyano, isocyano, unsubstituted or halogenated C1 to C12 linear or branched alkyl, C3 to C12 cycloalkyl, unsubstituted or halogenated C1 to C12 alkoxy, unsubstituted or halogenated C1 to C12 alkylthio, C6 to C20 aryl, C3 to C20 heteroaryl, C6 to C20 aryl ester or C6 to C20 arylsilyl.

In some optional embodiments of the present disclosure, Ar₁ and Ar₂ are each independently selected from any one of the following substituted or unsubstituted groups:

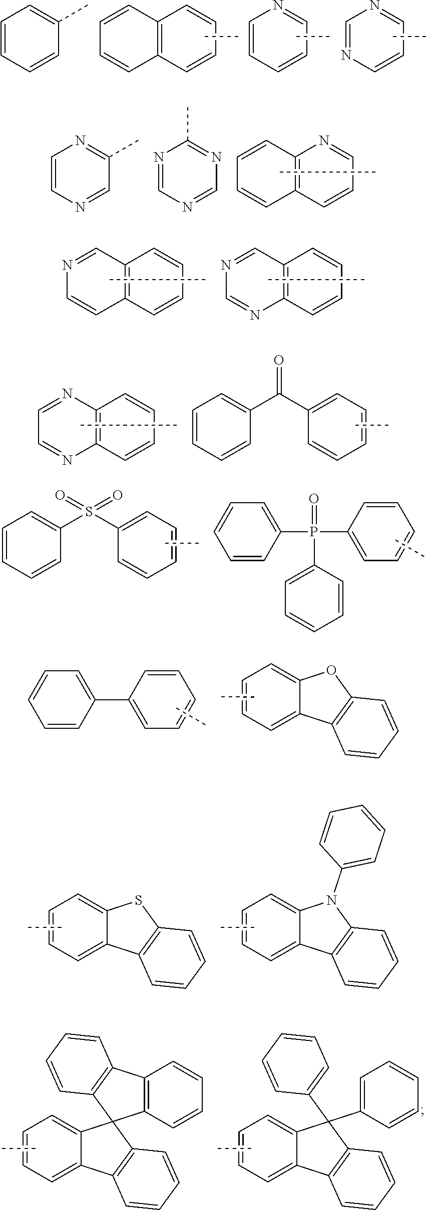

where the dashed line represents a linkage site of the group.

Substituents for substitutions in Ar₁ and Ar₂ are each independently selected from at least one of deuterium, halogen, cyano, isocyano, unsubstituted or halogenated C1 to C12 (for example, C1, C2, C3, C4, C5, C6, C7, C8, C9, C10, C11 or C12) linear or branched alkyl, C3 to C12 (for example, C3, C4, C5, C6, C7, C8, C9, C10, C11 or C12) cycloalkyl, unsubstituted or halogenated C1 to C12 (for example, C1, C2, C3, C4, C5, C6, C7, C8, C9, C10, C11 or C12) alkoxy, unsubstituted or halogenated C1 to C12 (for example, C1, C2, C3, C4, C5, C6, C7, C8, C9, C10, C11 or C12) alkylthio, C6 to C20 (for example, C6, C8, C9, C10, C12, C13, C14, C15, C16, C18 or the like) aryl, C6 to C20 (for example, C6, C8, C9, C10, C12, C13, C14, C15, C16, C18 or the like) aryl ester or C6 to C20 (for example, C6, C8, C9, C10, C12, C13, C14, C15, C16, C18 or the like) arylsilyl.

In some optional embodiments of the present disclosure, the substituents for the substitutions in Ar₁ and Ar₂ are each independently selected from at least one of deuterium, fluorine, cyano, isocyano, unsubstituted or fluorinated C1 to C6 (for example, C1, C2, C3, C4, C5 or C6) linear or branched alkyl, cyclohexyl, adamantyl, unsubstituted or fluorinated C1 to C6 (for example, C1, C2, C3, C4, C5 or C6) alkoxy, phenyl, naphthyl, phenyl ester or triphenylsilyl.

In some optional embodiments of the present disclosure, at least one of Ar₁ or Ar₂ includes an electron withdrawing group.

In the present disclosure, the term “electron withdrawing group” refers to a group capable of reducing an electron cloud density of a benzene ring. The electron withdrawing group exemplarily includes, but is not limited to, C6 to C30 aryl substituted with halogen, C6 to C30 aryl substituted with haloalkyl (halogenated C1 to C12 linear or branched alkyl), C6 to C30 aryl substituted with haloalkoxy (halogenated C1 to C12 alkoxy), C3 to C30 nitrogen-containing heteroaryl, C6 to C30 ketoaryl, C3 to C30 ketoheteroaryl, C6 to C30 aryl sulfonyl, C3 to C30 heteroaryl sulfonyl, C6 to C30 aryl phosphorus oxygen or C3 to C30 heteroaryl phosphorus oxygen.

In the present disclosure, at least one (one or two) of Ar₁ or Ar₂ including the electron withdrawing group is conductive to increasing a molecular polarization ratio, regulating the refractive index of the organic compound and making the difference between the refractive indexes of the organic compound in the different light colors smaller, thereby improving the color cast.

In some optional embodiments of the present disclosure, at least one (one or two) of Ar₁ or Ar₂ is selected from any one of the following groups:

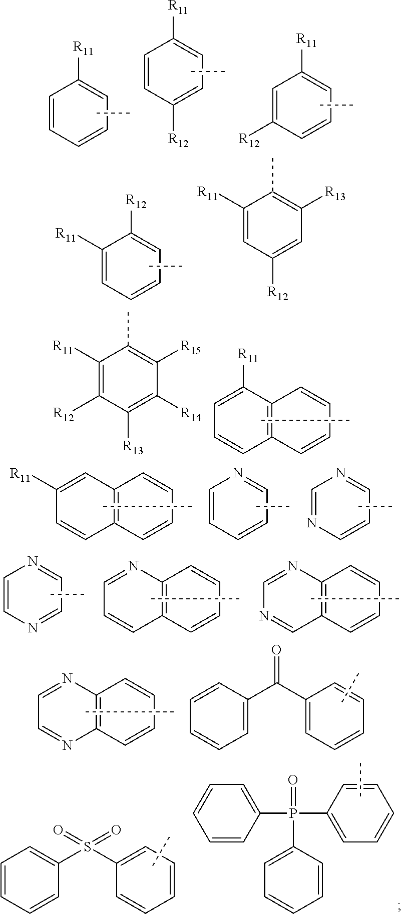

where the dashed line represents a linkage site of the group.

R₁₁, R₁₂, R₁₃, R₁₄ and R₁₅ are each independently selected from any one of fluorine, cyano, isocyano, fluorinated C1 to C6 (for example, C1, C2, C3, C4, C5 or C6) linear or branched alkyl or fluorinated C1 to C6 (for example, C1, C2, C3, C4, C5 or C6) alkoxy.

In some optional embodiments of the present disclosure, one of Ar₁ or Ar₂ is selected from any one of the following groups:

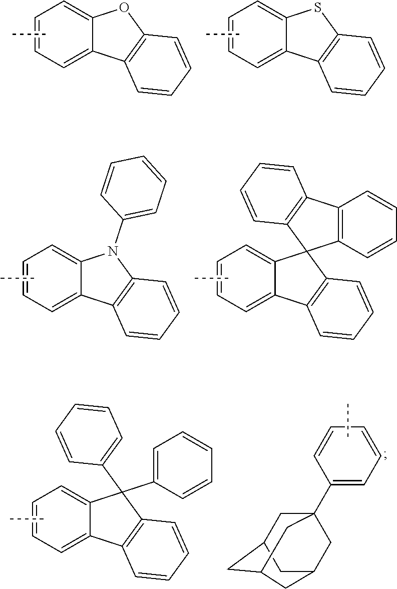

where the dashed line represents a linkage site of the group.

In some optional embodiments of the present disclosure, one of Ar₁ or Ar₂ is the above group with a relatively large steric hindrance that increases a volume of molecules and reduces the refractive index of the material. Moreover, the group is conducive to regulating a build-up structure and film-forming performance of the molecules, improving stability and enabling the organic compound to form a capping layer (CPL) with excellent performance through thermal evaporation, thereby improving the light extraction efficiency and luminescence efficiency of the element and improving the color cast.

In some optional embodiments, the organic compound is selected from any one of the following compounds P1 to P141:

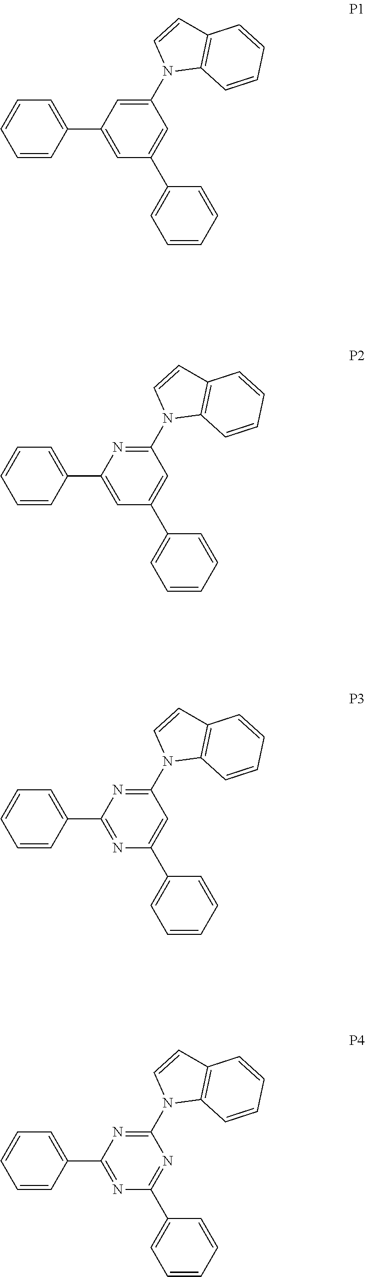 [SMILES: c1ccc(-c2cc(-c3ccccc3)cc(-n3ccc4ccccc43)c2)cc1 ; c1ccc(-c2cc(-c3ccccc3)nc(-n3ccc4ccccc43)c2)cc1 ; c1ccc(-c2cc(-n3ccc4ccccc43)nc(-c3ccccc3)n2)cc1 ; c1ccc(-c2nc(-c3ccccc3)nc(-n3ccc4ccccc43)n2)cc1]

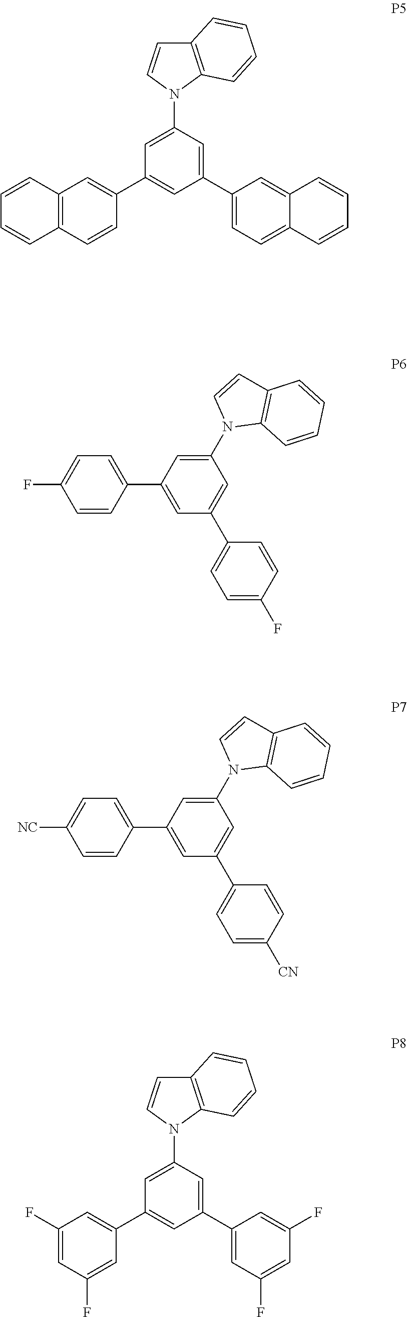 [SMILES: c1ccc2cc(-c3cc(-c4ccc5ccccc5c4)cc(-n4ccc5ccccc54)c3)ccc2c1 ; Fc1ccc(-c2cc(-c3ccc(F)cc3)cc(-n3ccc4ccccc43)c2)cc1 ; N#Cc1ccc(-c2cc(-c3ccc(C#N)cc3)cc(-n3ccc4ccccc43)c2)cc1 ; Fc1cc(F)cc(-c2cc(-c3cc(F)cc(F)c3)cc(-n3ccc4ccccc43)c2)c1]

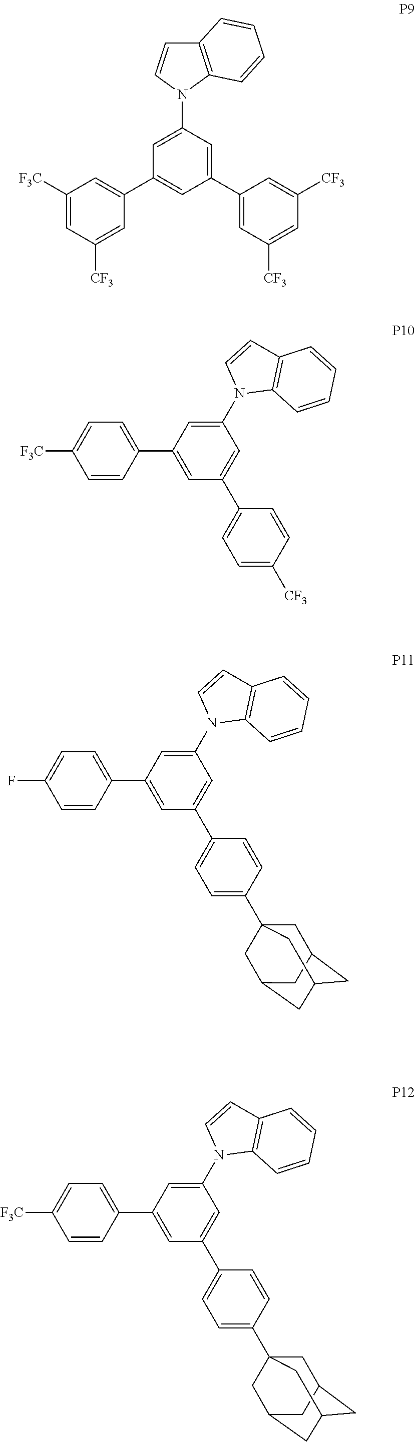 [SMILES: FC(F)(F)c1cc(-c2cc(-c3cc(C(F)(F)F)cc(C(F)(F)F)c3)cc(-n3ccc4ccccc43)c2)cc(C(F)(F)F)c1 ; FC(F)(F)c1ccc(-c2cc(-c3ccc(C(F)(F)F)cc3)cc(-n3ccc4ccccc43)c2)cc1 ; Fc1ccc(-c2cc(-c3ccc(C45CC6CC(CC(C6)C4)C5)cc3)cc(-n3ccc4ccccc43)c2)cc1 ; FC(F)(F)c1ccc(-c2cc(-c3ccc(C45CC6CC(CC(C6)C4)C5)cc3)cc(-n3ccc4ccccc43)c2)cc1]

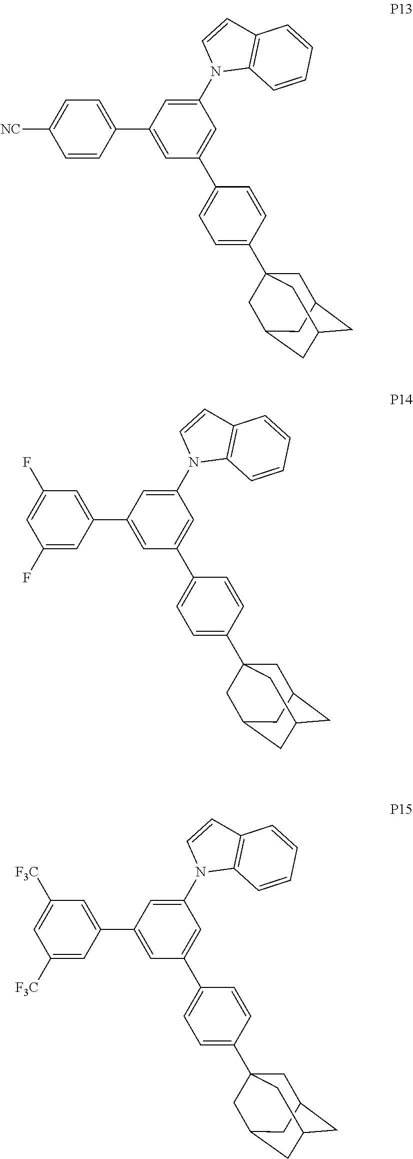 [SMILES: N#Cc1ccc(-c2cc(-c3ccc(C45CC6CC(CC(C6)C4)C5)cc3)cc(-n3ccc4ccccc43)c2)cc1 ; Fc1cc(F)cc(-c2cc(-c3ccc(C45CC6CC(CC(C6)C4)C5)cc3)cc(-n3ccc4ccccc43)c2)c1 ; FC(F)(F)c1cc(-c2cc(-c3ccc(C45CC6CC(CC(C6)C4)C5)cc3)cc(-n3ccc4ccccc43)c2)cc(C(F)(F)F)c1]

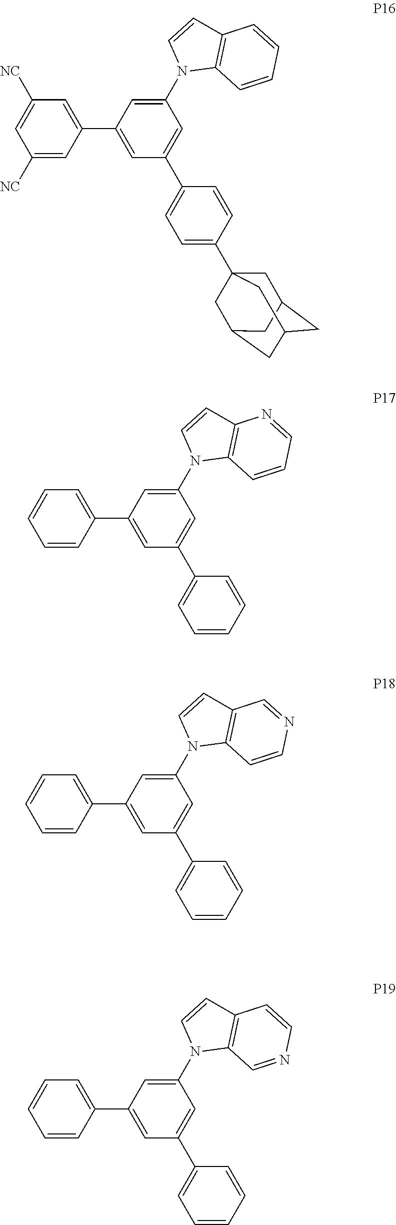 [SMILES: N#Cc1cc(C#N)cc(-c2cc(-c3ccc(C45CC6CC(CC(C6)C4)C5)cc3)cc(-n3ccc4ccccc43)c2)c1 ; c1ccc(-c2cc(-c3ccccc3)cc(-n3ccc4ncccc43)c2)cc1 ; c1ccc(-c2cc(-c3ccccc3)cc(-n3ccc4cnccc43)c2)cc1 ; c1ccc(-c2cc(-c3ccccc3)cc(-n3ccc4ccncc43)c2)cc1]

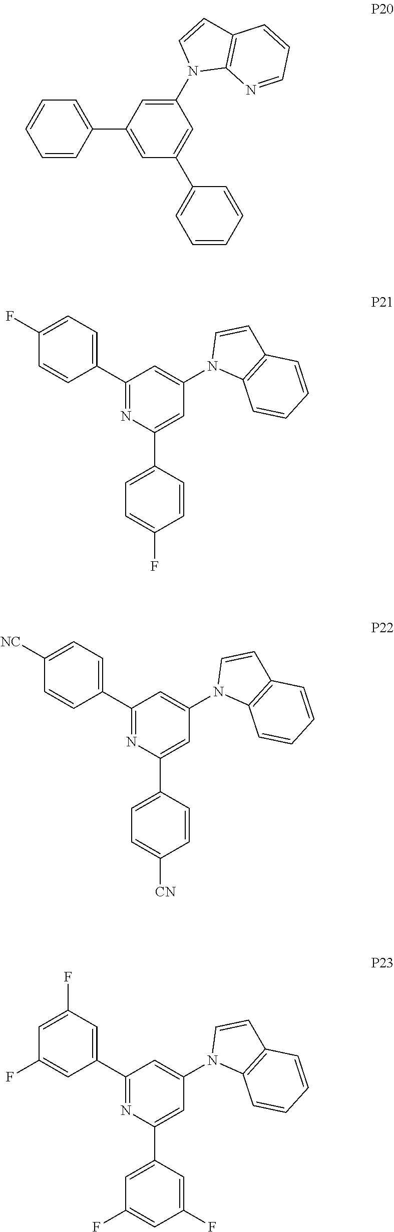 [SMILES: c1ccc(-c2cc(-c3ccccc3)cc(-n3ccc4cccnc43)c2)cc1 ; Fc1ccc(-c2cc(-n3ccc4ccccc43)cc(-c3ccc(F)cc3)n2)cc1 ; N#Cc1ccc(-c2cc(-n3ccc4ccccc43)cc(-c3ccc(C#N)cc3)n2)cc1 ; Fc1cc(F)cc(-c2cc(-n3ccc4ccccc43)cc(-c3cc(F)cc(F)c3)n2)c1]

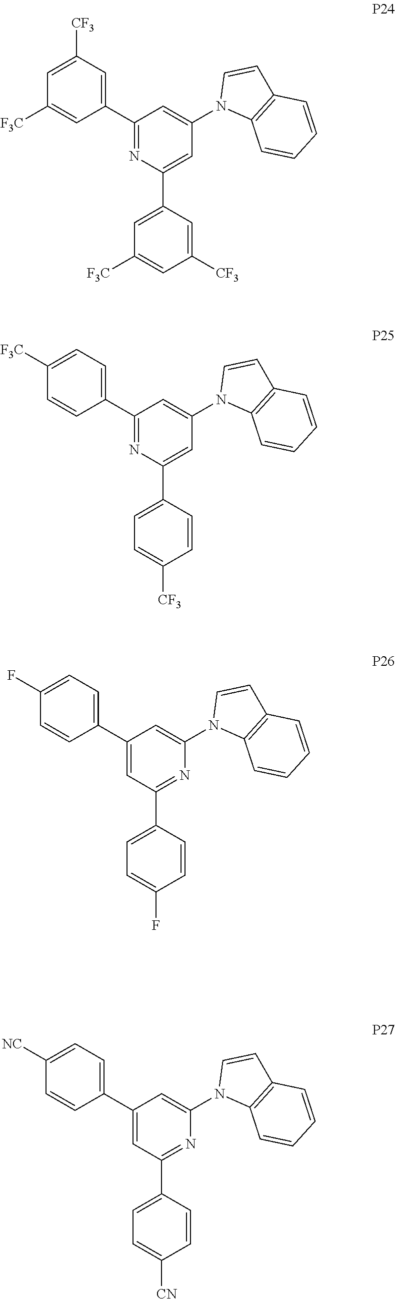 [SMILES: FC(F)(F)c1cc(-c2cc(-n3ccc4ccccc43)cc(-c3cc(C(F)(F)F)cc(C(F)(F)F)c3)n2)cc(C(F)(F)F)c1 ; FC(F)(F)c1ccc(-c2cc(-n3ccc4ccccc43)cc(-c3ccc(C(F)(F)F)cc3)n2)cc1 ; Fc1ccc(-c2cc(-c3ccc(F)cc3)nc(-n3ccc4ccccc43)c2)cc1 ; N#Cc1ccc(-c2cc(-c3ccc(C#N)cc3)nc(-n3ccc4ccccc43)c2)cc1]

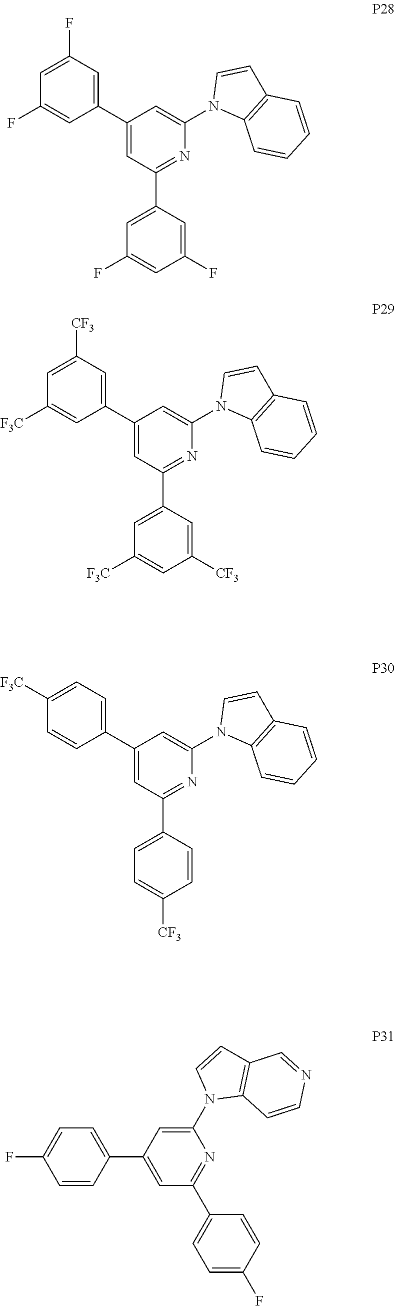 [SMILES: Fc1cc(F)cc(-c2cc(-c3cc(F)cc(F)c3)nc(-n3ccc4ccccc43)c2)c1 ; FC(F)(F)c1cc(-c2cc(-c3cc(C(F)(F)F)cc(C(F)(F)F)c3)nc(-n3ccc4ccccc43)c2)cc(C(F)(F)F)c1 ; FC(F)(F)c1ccc(-c2cc(-c3ccc(C(F)(F)F)cc3)nc(-n3ccc4ccccc43)c2)cc1 ; Fc1ccc(-c2cc(-c3ccc(F)cc3)nc(-n3ccc4cnccc43)c2)cc1]

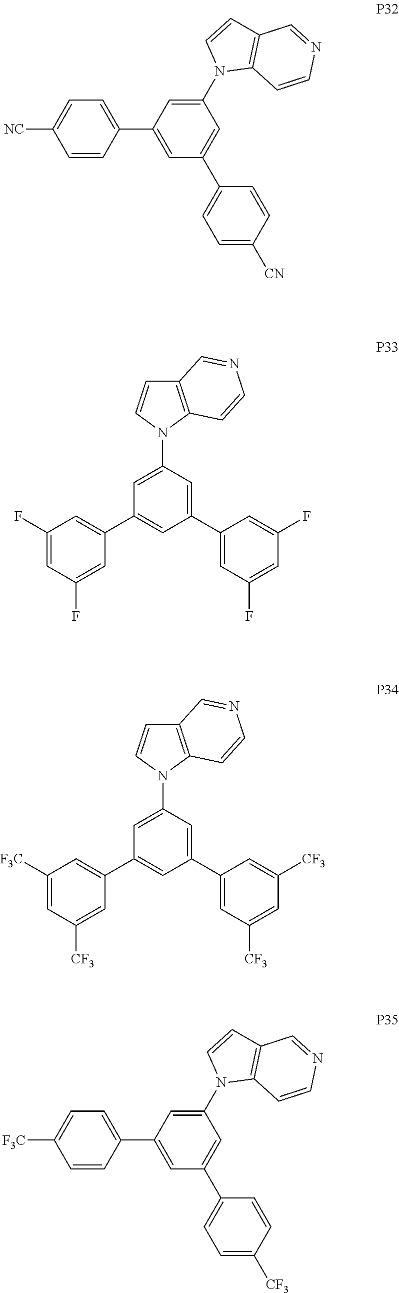 [SMILES: N#Cc1ccc(-c2cc(-c3ccc(C#N)cc3)cc(-n3ccc4cnccc43)c2)cc1 ; Fc1cc(F)cc(-c2cc(-c3cc(F)cc(F)c3)cc(-n3ccc4cnccc43)c2)c1 ; FC(F)(F)c1cc(-c2cc(-c3cc(C(F)(F)F)cc(C(F)(F)F)c3)cc(-n3ccc4cnccc43)c2)cc(C(F)(F)F)c1 ; FC(F)(F)c1ccc(-c2cc(-c3ccc(C(F)(F)F)cc3)cc(-n3ccc4cnccc43)c2)cc1]

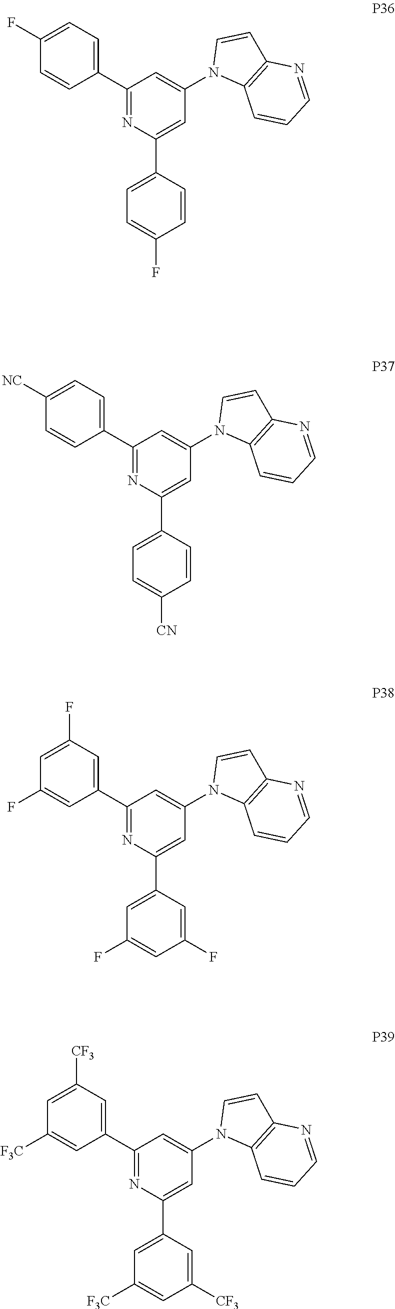 [SMILES: Fc1ccc(-c2cc(-n3ccc4ncccc43)cc(-c3ccc(F)cc3)n2)cc1 ; N#Cc1ccc(-c2cc(-n3ccc4ncccc43)cc(-c3ccc(C#N)cc3)n2)cc1 ; Fc1cc(F)cc(-c2cc(-n3ccc4ncccc43)cc(-c3cc(F)cc(F)c3)n2)c1 ; FC(F)(F)c1cc(-c2cc(-n3ccc4ncccc43)cc(-c3cc(C(F)(F)F)cc(C(F)(F)F)c3)n2)cc(C(F)(F)F)c1]

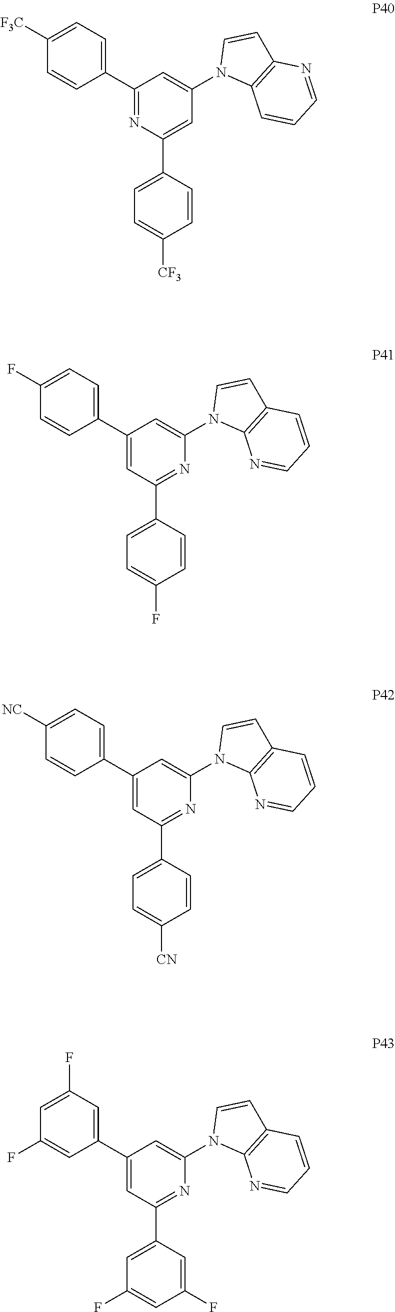 [SMILES: FC(F)(F)c1ccc(-c2cc(-n3ccc4ncccc43)cc(-c3ccc(C(F)(F)F)cc3)n2)cc1 ; Fc1ccc(-c2cc(-c3ccc(F)cc3)nc(-n3ccc4cccnc43)c2)cc1 ; N#Cc1ccc(-c2cc(-c3ccc(C#N)cc3)nc(-n3ccc4cccnc43)c2)cc1 ; Fc1cc(F)cc(-c2cc(-c3cc(F)cc(F)c3)nc(-n3ccc4cccnc43)c2)c1]

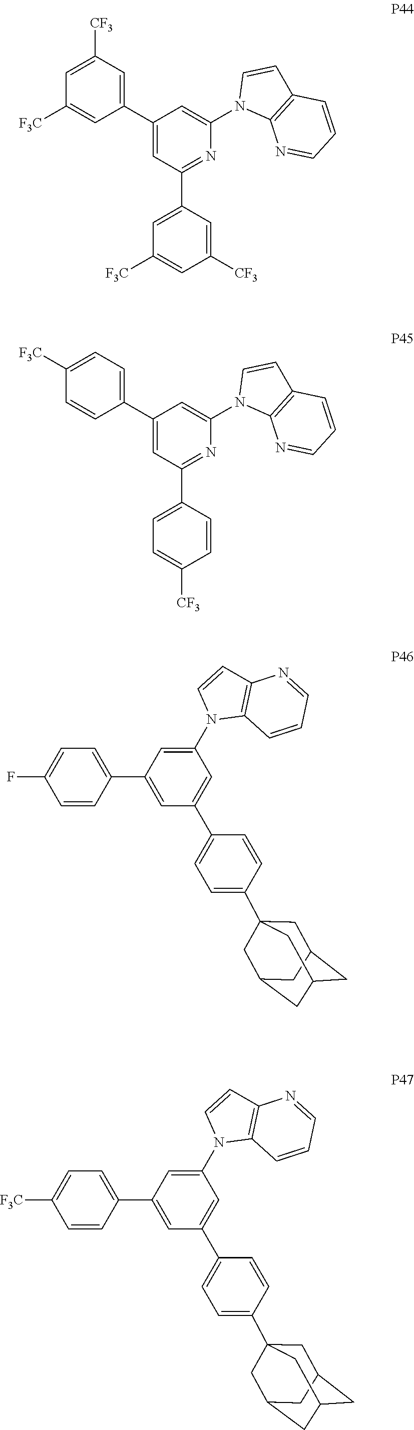 [SMILES: FC(F)(F)c1cc(-c2cc(-c3cc(C(F)(F)F)cc(C(F)(F)F)c3)nc(-n3ccc4cccnc43)c2)cc(C(F)(F)F)c1 ; FC(F)(F)c1ccc(-c2cc(-c3ccc(C(F)(F)F)cc3)nc(-n3ccc4cccnc43)c2)cc1 ; Fc1ccc(-c2cc(-c3ccc(C45CC6CC(CC(C6)C4)C5)cc3)cc(-n3ccc4ncccc43)c2)cc1 ; FC(F)(F)c1ccc(-c2cc(-c3ccc(C45CC6CC(CC(C6)C4)C5)cc3)cc(-n3ccc4ncccc43)c2)cc1]

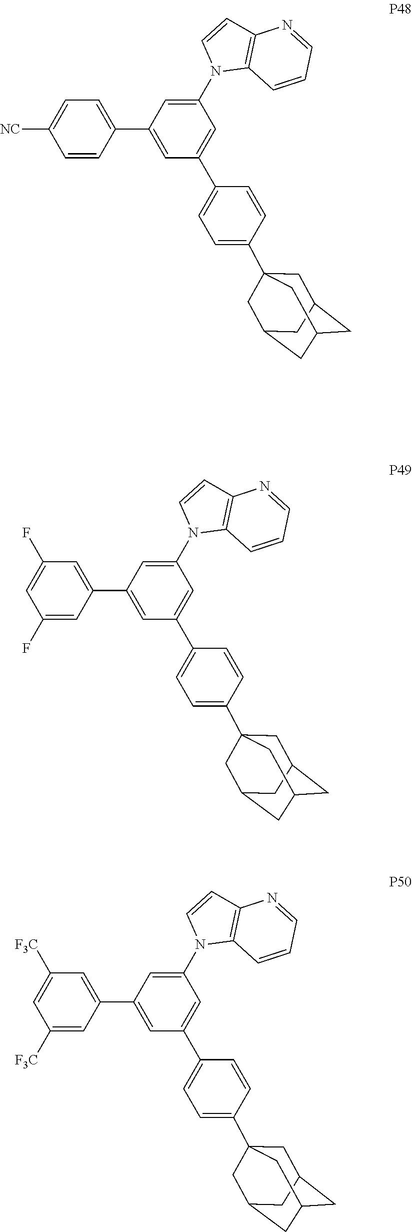 [SMILES: N#Cc1ccc(-c2cc(-c3ccc(C45CC6CC(CC(C6)C4)C5)cc3)cc(-n3ccc4ncccc43)c2)cc1 ; Fc1cc(F)cc(-c2cc(-c3ccc(C45CC6CC(CC(C6)C4)C5)cc3)cc(-n3ccc4ncccc43)c2)c1 ; FC(F)(F)c1cc(-c2cc(-c3ccc(C45CC6CC(CC(C6)C4)C5)cc3)cc(-n3ccc4ncccc43)c2)cc(C(F)(F)F)c1]

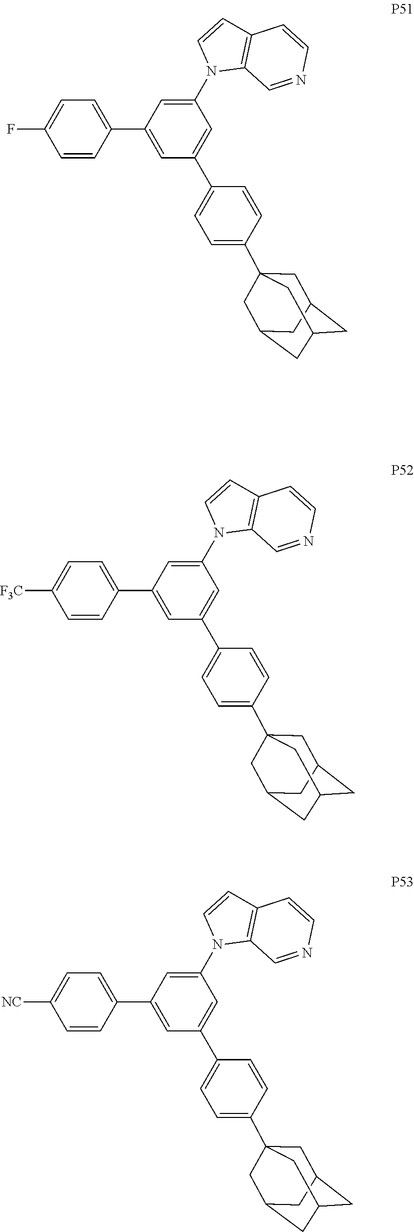 [SMILES: Fc1ccc(-c2cc(-c3ccc(C45CC6CC(CC(C6)C4)C5)cc3)cc(-n3ccc4ccncc43)c2)cc1 ; FC(F)(F)c1ccc(-c2cc(-c3ccc(C45CC6CC(CC(C6)C4)C5)cc3)cc(-n3ccc4ccncc43)c2)cc1 ; N#Cc1ccc(-c2cc(-c3ccc(C45CC6CC(CC(C6)C4)C5)cc3)cc(-n3ccc4ccncc43)c2)cc1]

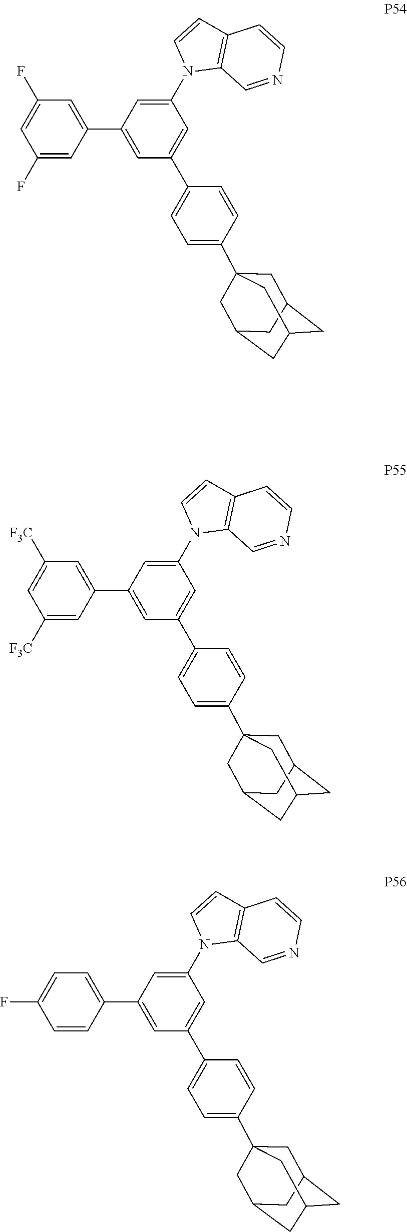 [SMILES: Fc1cc(F)cc(-c2cc(-c3ccc(C45CC6CC(CC(C6)C4)C5)cc3)cc(-n3ccc4ccncc43)c2)c1 ; FC(F)(F)c1cc(-c2cc(-c3ccc(C45CC6CC(CC(C6)C4)C5)cc3)cc(-n3ccc4ccncc43)c2)cc(C(F)(F)F)c1 ; Fc1ccc(-c2cc(-c3ccc(C45CC6CC(CC(C6)C4)C5)cc3)cc(-n3ccc4ccncc43)c2)cc1]

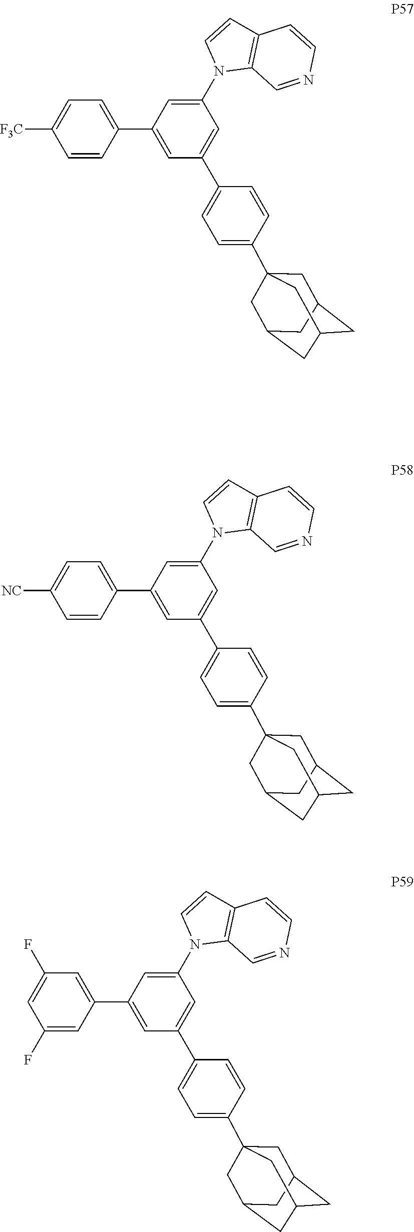 [SMILES: FC(F)(F)c1ccc(-c2cc(-c3ccc(C45CC6CC(CC(C6)C4)C5)cc3)cc(-n3ccc4ccncc43)c2)cc1 ; N#Cc1ccc(-c2cc(-c3ccc(C45CC6CC(CC(C6)C4)C5)cc3)cc(-n3ccc4ccncc43)c2)cc1 ; Fc1cc(F)cc(-c2cc(-c3ccc(C45CC6CC(CC(C6)C4)C5)cc3)cc(-n3ccc4ccncc43)c2)c1]

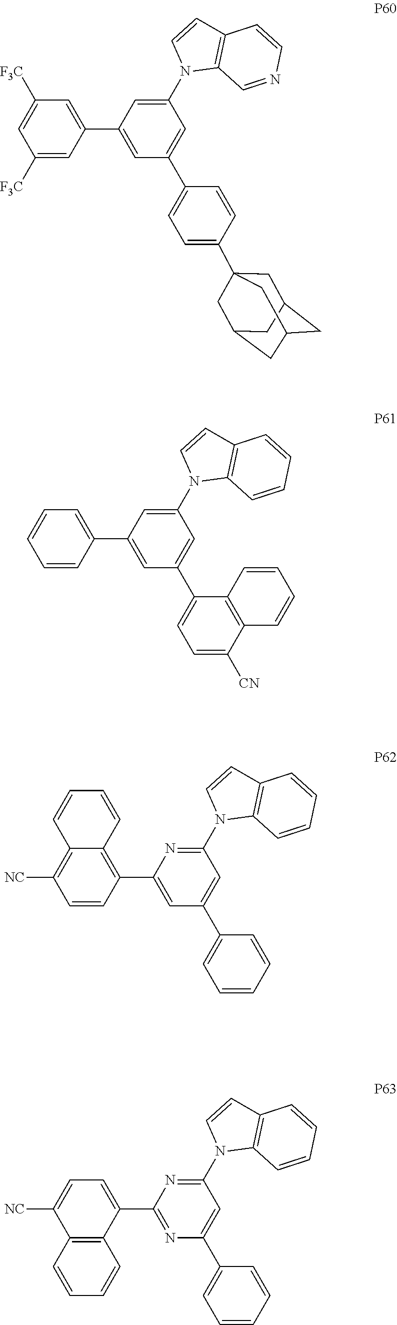 [SMILES: FC(F)(F)c1cc(-c2cc(-c3ccc(C45CC6CC(CC(C6)C4)C5)cc3)cc(-n3ccc4ccncc43)c2)cc(C(F)(F)F)c1 ; N#Cc1ccc(-c2cc(-c3ccccc3)cc(-n3ccc4ccccc43)c2)c2ccccc12 ; N#Cc1ccc(-c2cc(-c3ccccc3)cc(-n3ccc4ccccc43)n2)c2ccccc12 ; N#Cc1ccc(-c2nc(-c3ccccc3)cc(-n3ccc4ccccc43)n2)c2ccccc12]

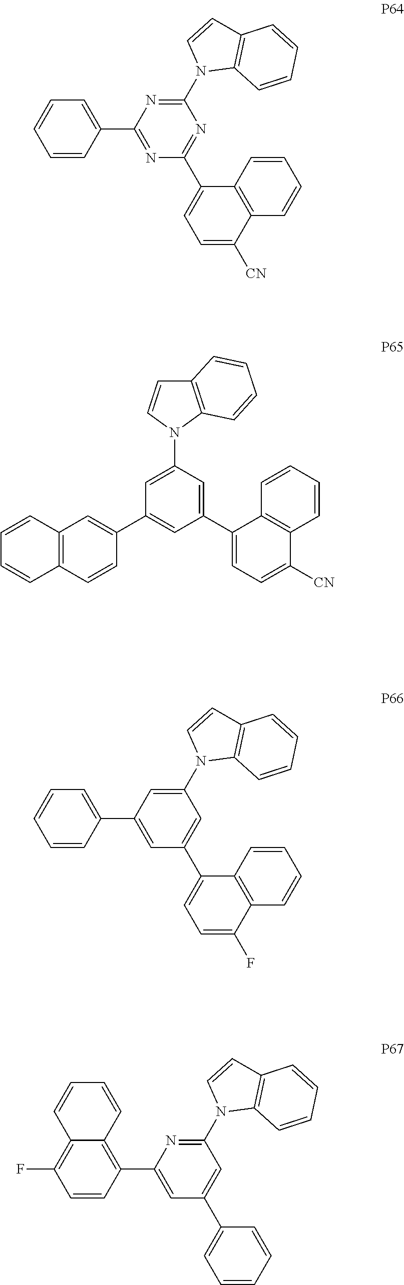 [SMILES: N#Cc1ccc(-c2nc(-c3ccccc3)nc(-n3ccc4ccccc43)n2)c2ccccc12 ; N#Cc1ccc(-c2cc(-c3ccc4ccccc4c3)cc(-n3ccc4ccccc43)c2)c2ccccc12 ; Fc1ccc(-c2cc(-c3ccccc3)cc(-n3ccc4ccccc43)c2)c2ccccc12 ; Fc1ccc(-c2cc(-c3ccccc3)cc(-n3ccc4ccccc43)n2)c2ccccc12]

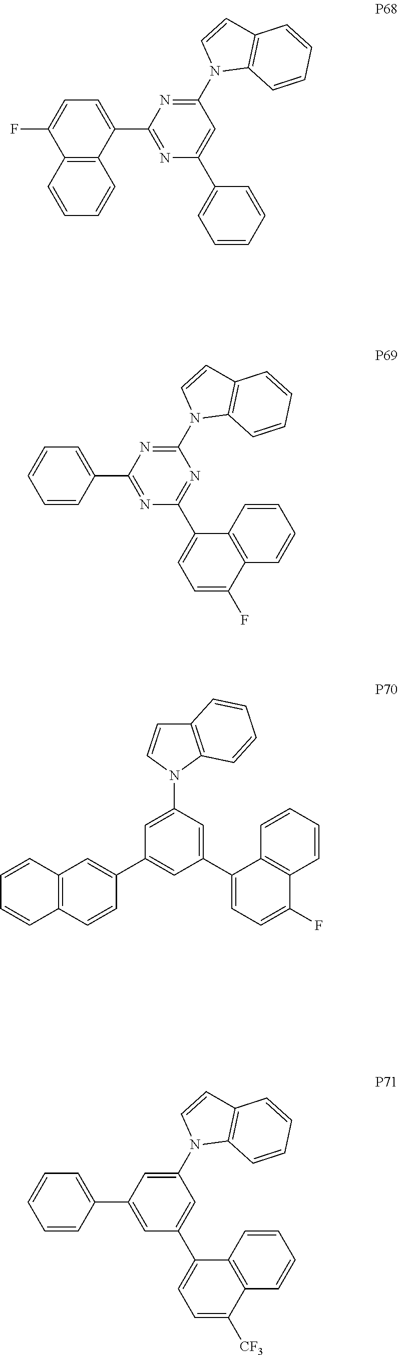 [SMILES: Fc1ccc(-c2nc(-c3ccccc3)cc(-n3ccc4ccccc43)n2)c2ccccc12 ; Fc1ccc(-c2nc(-c3ccccc3)nc(-n3ccc4ccccc43)n2)c2ccccc12 ; Fc1ccc(-c2cc(-c3ccc4ccccc4c3)cc(-n3ccc4ccccc43)c2)c2ccccc12 ; FC(F)(F)c1ccc(-c2cc(-c3ccccc3)cc(-n3ccc4ccccc43)c2)c2ccccc12]

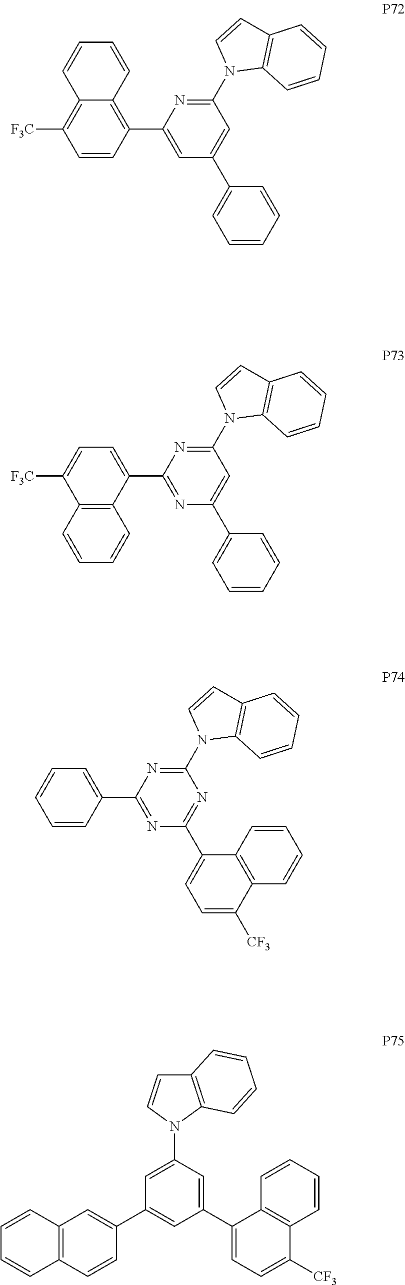 [SMILES: FC(F)(F)c1ccc(-c2cc(-c3ccccc3)cc(-n3ccc4ccccc43)n2)c2ccccc12 ; FC(F)(F)c1ccc(-c2nc(-c3ccccc3)cc(-n3ccc4ccccc43)n2)c2ccccc12 ; FC(F)(F)c1ccc(-c2nc(-c3ccccc3)nc(-n3ccc4ccccc43)n2)c2ccccc12 ; FC(F)(F)c1ccc(-c2cc(-c3ccc4ccccc4c3)cc(-n3ccc4ccccc43)c2)c2ccccc12]

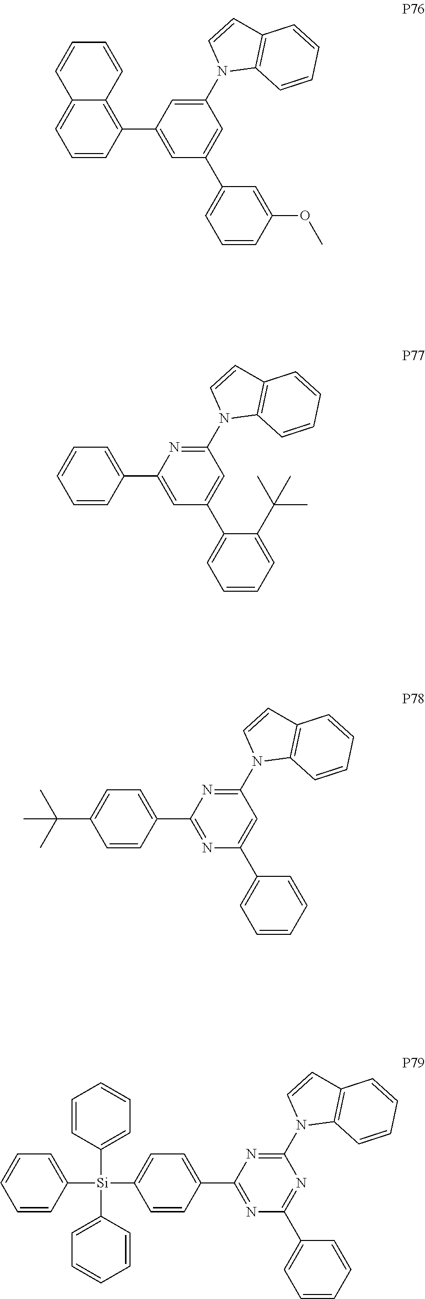

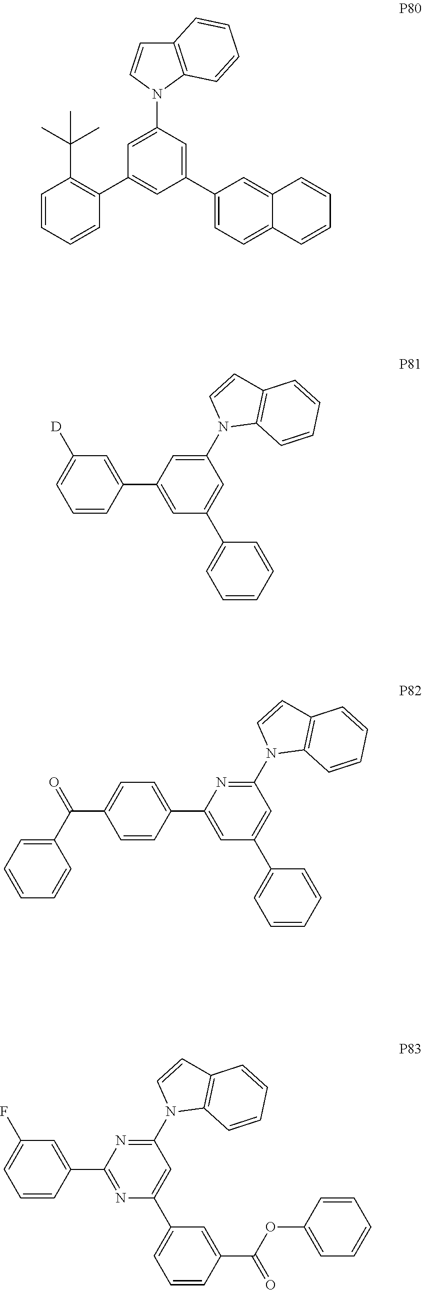 [SMILES: CC(C)(C)c1ccccc1-c1cc(-c2ccc3ccccc3c2)cc(-n2ccc3ccccc32)c1 ; [2H]c1cccc(-c2cc(-c3ccccc3)cc(-n3ccc4ccccc43)c2)c1 ; O=C(c1ccccc1)c1ccc(-c2cc(-c3ccccc3)cc(-n3ccc4ccccc43)n2)cc1 ; O=C(Oc1ccccc1)c1cccc(-c2cc(-n3ccc4ccccc43)nc(-c3cccc(F)c3)n2)c1]

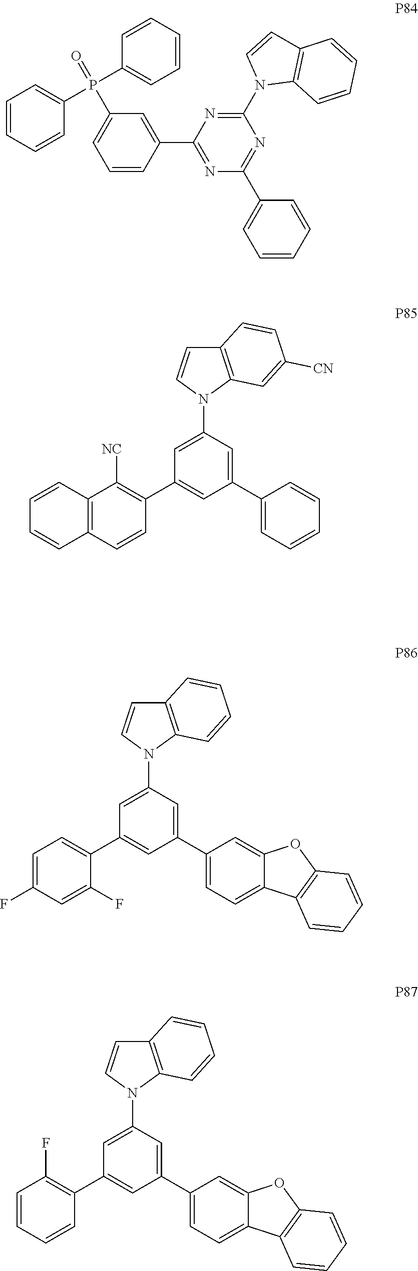 [SMILES: O=P(c1ccccc1)(c1ccccc1)c1cccc(-c2nc(-c3ccccc3)nc(-n3ccc4ccccc43)n2)c1 ; N#Cc1ccc2ccn(-c3cc(-c4ccccc4)cc(-c4ccc5ccccc5c4C#N)c3)c2c1 ; Fc1ccc(-c2cc(-c3ccc4c(c3)oc3ccccc34)cc(-n3ccc4ccccc43)c2)c(F)c1 ; Fc1ccccc1-c1cc(-c2ccc3c(c2)oc2ccccc23)cc(-n2ccc3ccccc32)c1]

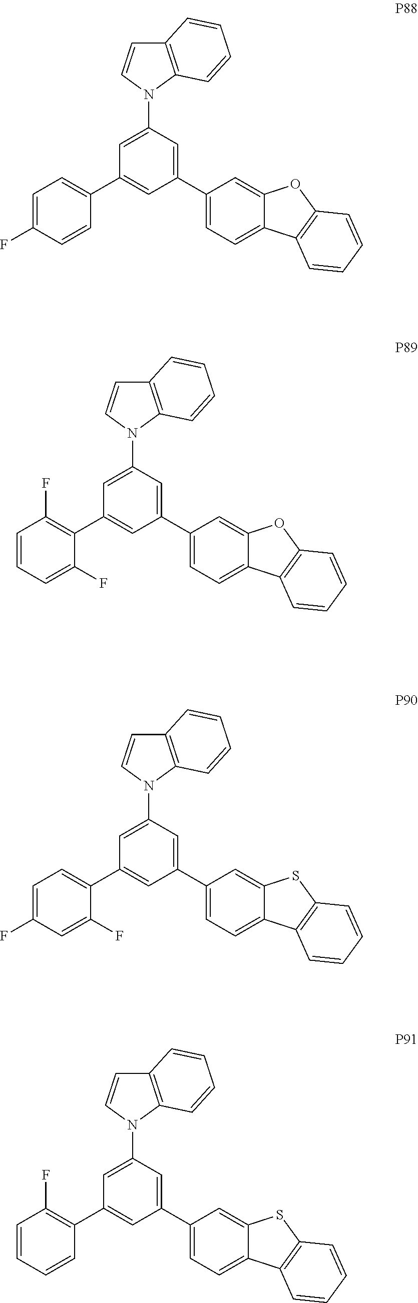 [SMILES: Fc1ccc(-c2cc(-c3ccc4c(c3)oc3ccccc34)cc(-n3ccc4ccccc43)c2)cc1 ; Fc1cccc(F)c1-c1cc(-c2ccc3c(c2)oc2ccccc23)cc(-n2ccc3ccccc32)c1 ; Fc1ccc(-c2cc(-c3ccc4c(c3)sc3ccccc34)cc(-n3ccc4ccccc43)c2)c(F)c1 ; Fc1ccccc1-c1cc(-c2ccc3c(c2)sc2ccccc23)cc(-n2ccc3ccccc32)c1]

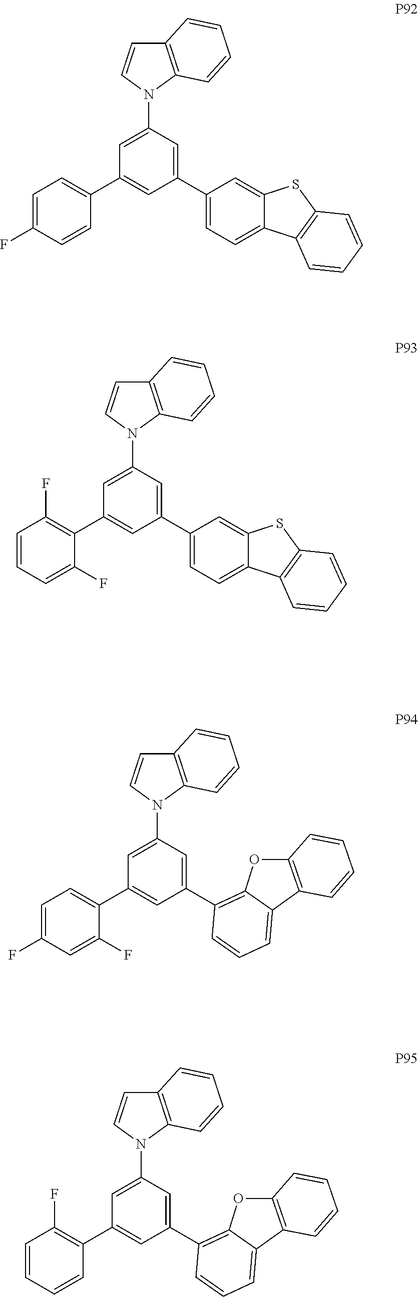 [SMILES: Fc1ccc(-c2cc(-c3ccc4c(c3)sc3ccccc34)cc(-n3ccc4ccccc43)c2)cc1 ; Fc1cccc(F)c1-c1cc(-c2ccc3c(c2)sc2ccccc23)cc(-n2ccc3ccccc32)c1 ; Fc1ccc(-c2cc(-c3cccc4c3oc3ccccc34)cc(-n3ccc4ccccc43)c2)c(F)c1 ; Fc1ccccc1-c1cc(-c2cccc3c2oc2ccccc23)cc(-n2ccc3ccccc32)c1]

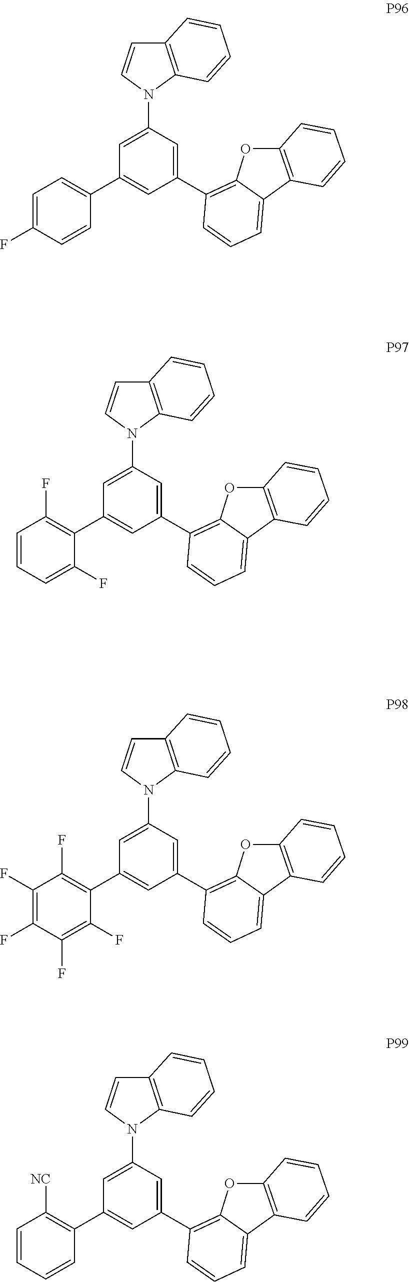 [SMILES: Fc1ccc(-c2cc(-c3cccc4c3oc3ccccc34)cc(-n3ccc4ccccc43)c2)cc1 ; Fc1cccc(F)c1-c1cc(-c2cccc3c2oc2ccccc23)cc(-n2ccc3ccccc32)c1 ; Fc1c(F)c(F)c(-c2cc(-c3cccc4c3oc3ccccc34)cc(-n3ccc4ccccc43)c2)c(F)c1F ; N#Cc1ccccc1-c1cc(-c2cccc3c2oc2ccccc23)cc(-n2ccc3ccccc32)c1]

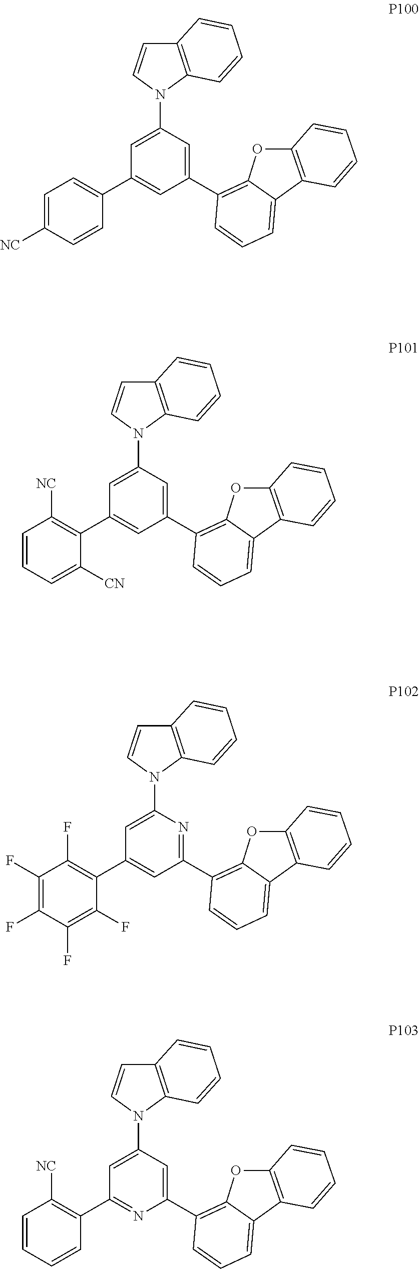 [SMILES: N#Cc1ccc(-c2cc(-c3cccc4c3oc3ccccc34)cc(-n3ccc4ccccc43)c2)cc1 ; N#Cc1cccc(C#N)c1-c1cc(-c2cccc3c2oc2ccccc23)cc(-n2ccc3ccccc32)c1 ; Fc1c(F)c(F)c(-c2cc(-c3cccc4c3oc3ccccc34)nc(-n3ccc4ccccc43)c2)c(F)c1F ; N#Cc1ccccc1-c1cc(-n2ccc3ccccc32)cc(-c2cccc3c2oc2ccccc23)n1]

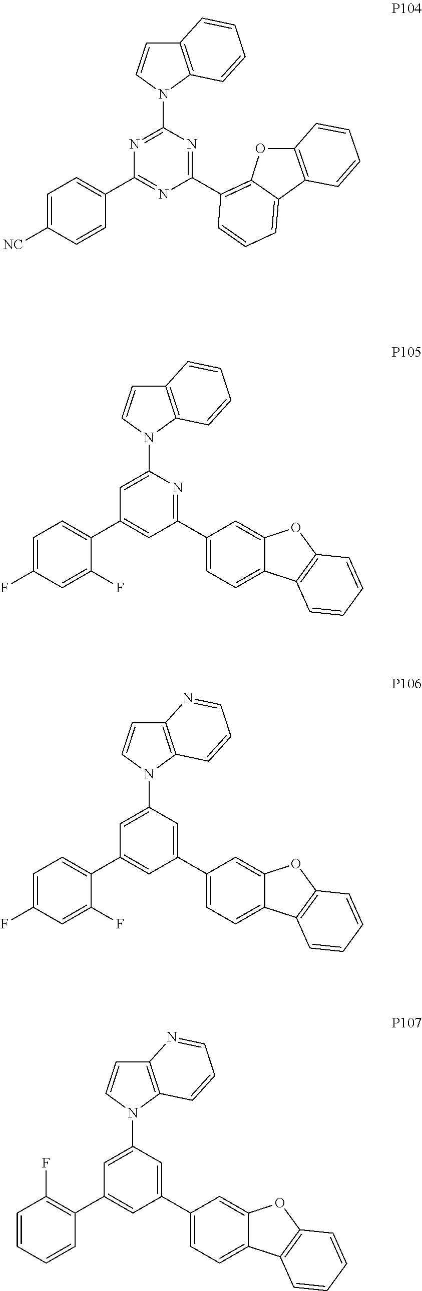 [SMILES: N#Cc1ccc(-c2nc(-c3cccc4c3oc3ccccc34)nc(-n3ccc4ccccc43)n2)cc1 ; Fc1ccc(-c2cc(-c3ccc4c(c3)oc3ccccc34)nc(-n3ccc4ccccc43)c2)c(F)c1 ; Fc1ccc(-c2cc(-c3ccc4c(c3)oc3ccccc34)cc(-n3ccc4ncccc43)c2)c(F)c1 ; Fc1ccccc1-c1cc(-c2ccc3c(c2)oc2ccccc23)cc(-n2ccc3ncccc32)c1]

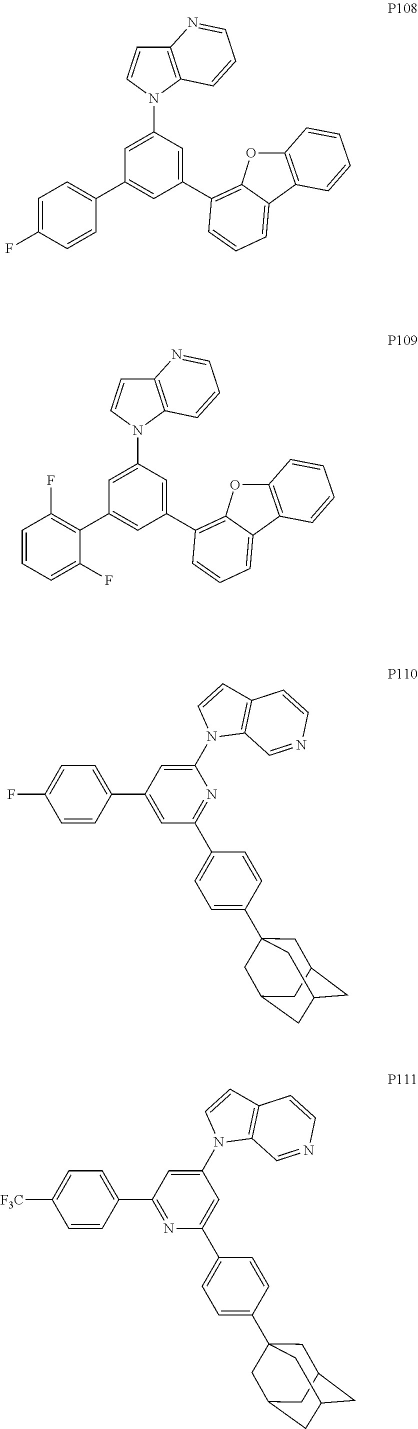 [SMILES: Fc1ccc(-c2cc(-c3cccc4c3oc3ccccc34)cc(-n3ccc4ncccc43)c2)cc1 ; Fc1cccc(F)c1-c1cc(-c2cccc3c2oc2ccccc23)cc(-n2ccc3ncccc32)c1 ; Fc1ccc(-c2cc(-c3ccc(C45CC6CC(CC(C6)C4)C5)cc3)nc(-n3ccc4ccncc43)c2)cc1 ; FC(F)(F)c1ccc(-c2cc(-n3ccc4ccncc43)cc(-c3ccc(C45CC6CC(CC(C6)C4)C5)cc3)n2)cc1]

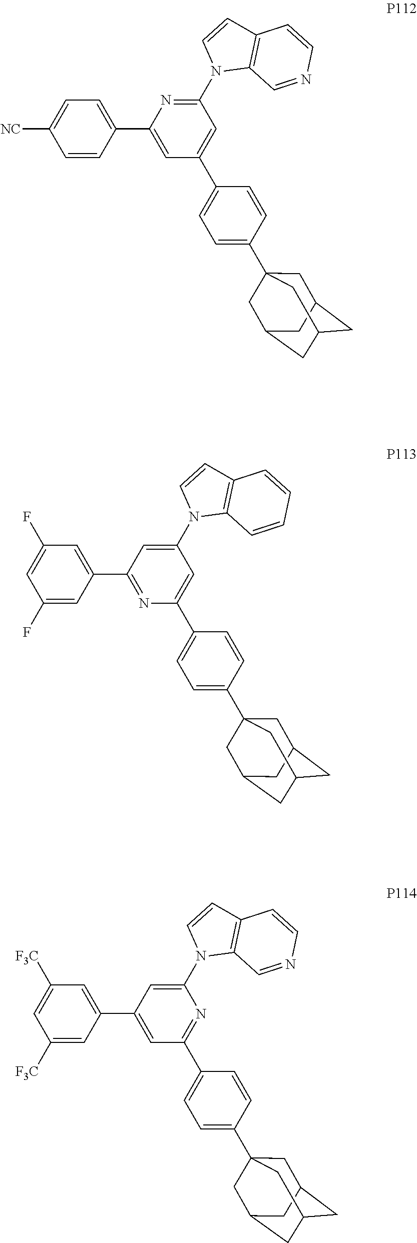 [SMILES: N#Cc1ccc(-c2cc(-c3ccc(C45CC6CC(CC(C6)C4)C5)cc3)cc(-n3ccc4ccncc43)n2)cc1 ; Fc1cc(F)cc(-c2cc(-n3ccc4ccccc43)cc(-c3ccc(C45CC6CC(CC(C6)C4)C5)cc3)n2)c1 ; FC(F)(F)c1cc(-c2cc(-c3ccc(C45CC6CC(CC(C6)C4)C5)cc3)nc(-n3ccc4ccncc43)c2)cc(C(F)(F)F)c1]

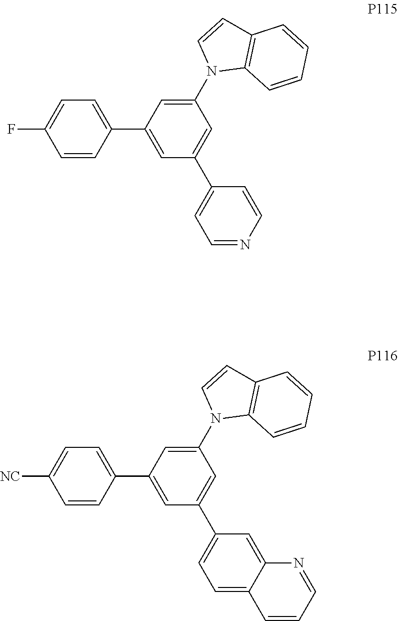 [SMILES: Fc1ccc(-c2cc(-c3ccncc3)cc(-n3ccc4ccccc43)c2)cc1 ; N#Cc1ccc(-c2cc(-c3ccc4cccnc4c3)cc(-n3ccc4ccccc43)c2)cc1]

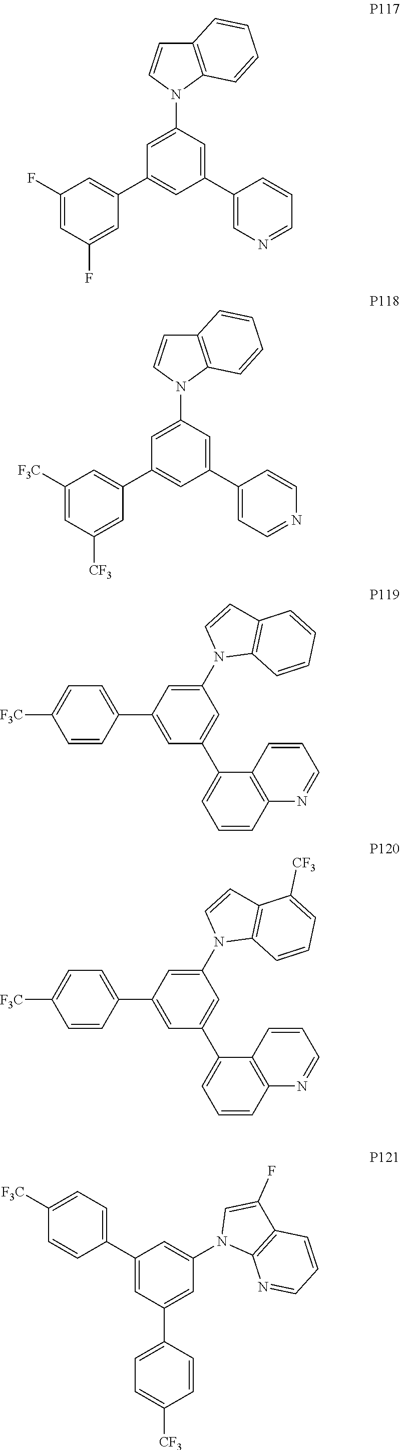 [SMILES: Fc1cc(F)cc(-c2cc(-c3cccnc3)cc(-n3ccc4ccccc43)c2)c1 ; FC(F)(F)c1cc(-c2cc(-c3ccncc3)cc(-n3ccc4ccccc43)c2)cc(C(F)(F)F)c1 ; FC(F)(F)c1ccc(-c2cc(-c3cccc4ncccc34)cc(-n3ccc4ccccc43)c2)cc1 ; FC(F)(F)c1ccc(-c2cc(-c3cccc4ncccc34)cc(-n3ccc4c(C(F)(F)F)cccc43)c2)cc1 ; Fc1cn(-c2cc(-c3ccc(C(F)(F)F)cc3)cc(-c3ccc(C(F)(F)F)cc3)c2)c2ncccc12]

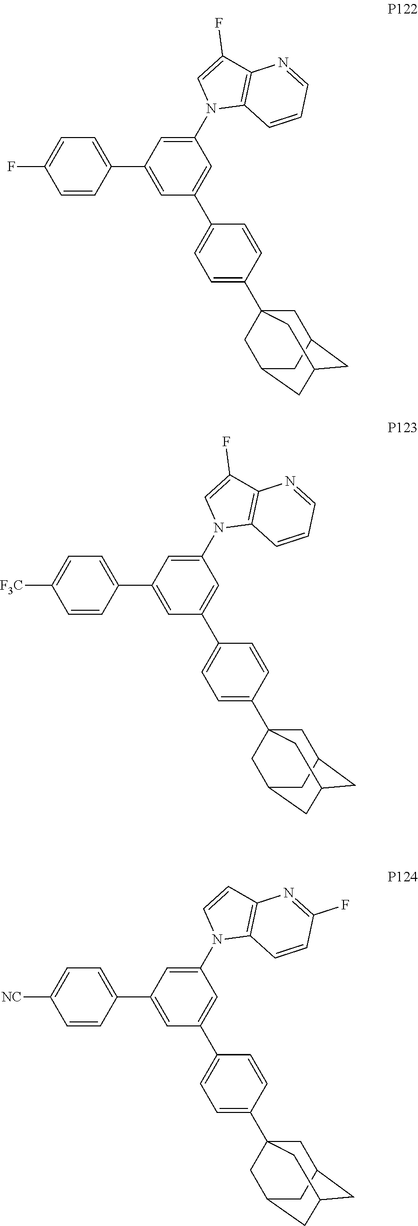 [SMILES: Fc1ccc(-c2cc(-c3ccc(C45CC6CC(CC(C6)C4)C5)cc3)cc(-n3cc(F)c4ncccc43)c2)cc1 ; Fc1cn(-c2cc(-c3ccc(C(F)(F)F)cc3)cc(-c3ccc(C45CC6CC(CC(C6)C4)C5)cc3)c2)c2cccnc12 ; N#Cc1ccc(-c2cc(-c3ccc(C45CC6CC(CC(C6)C4)C5)cc3)cc(-n3ccc4nc(F)ccc43)c2)cc1]

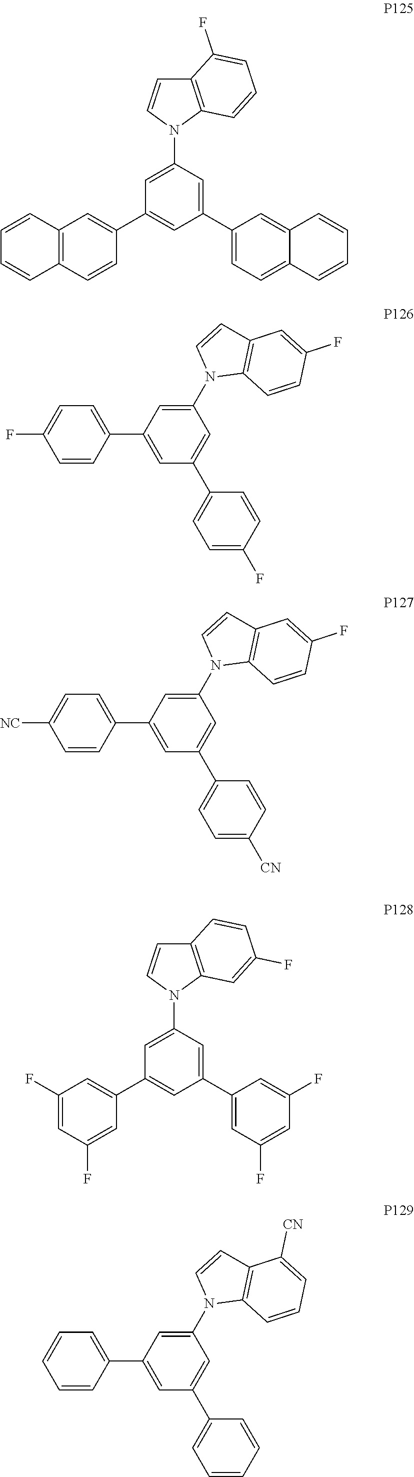 [SMILES: Fc1cccc2c1ccn2-c1cc(-c2ccc3ccccc3c2)cc(-c2ccc3ccccc3c2)c1 ; Fc1ccc(-c2cc(-c3ccc(F)cc3)cc(-n3ccc4cc(F)ccc43)c2)cc1 ; N#Cc1ccc(-c2cc(-c3ccc(C#N)cc3)cc(-n3ccc4cc(F)ccc43)c2)cc1 ; Fc1cc(F)cc(-c2cc(-c3cc(F)cc(F)c3)cc(-n3ccc4ccc(F)cc43)c2)c1 ; N#Cc1cccc2c1ccn2-c1cc(-c2ccccc2)cc(-c2ccccc2)c1]

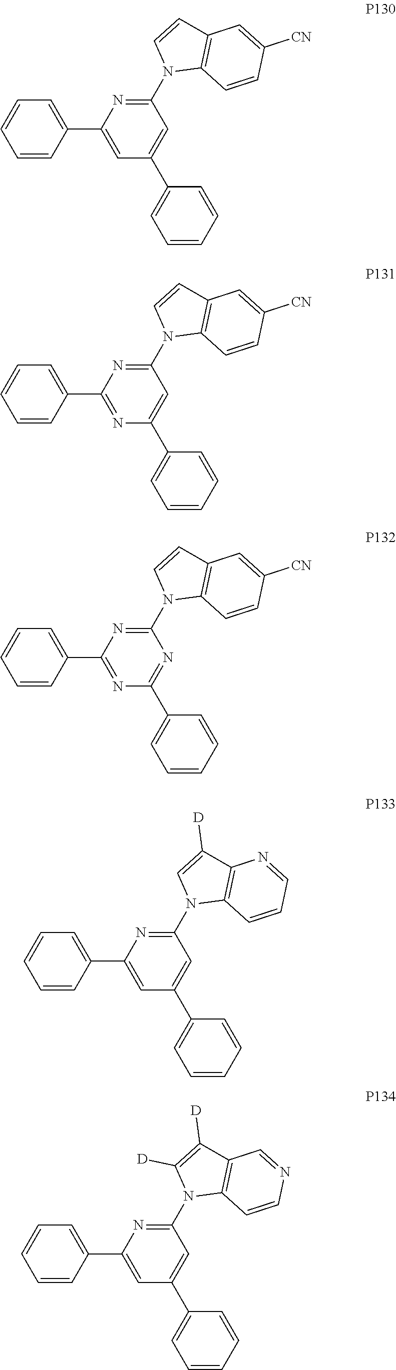

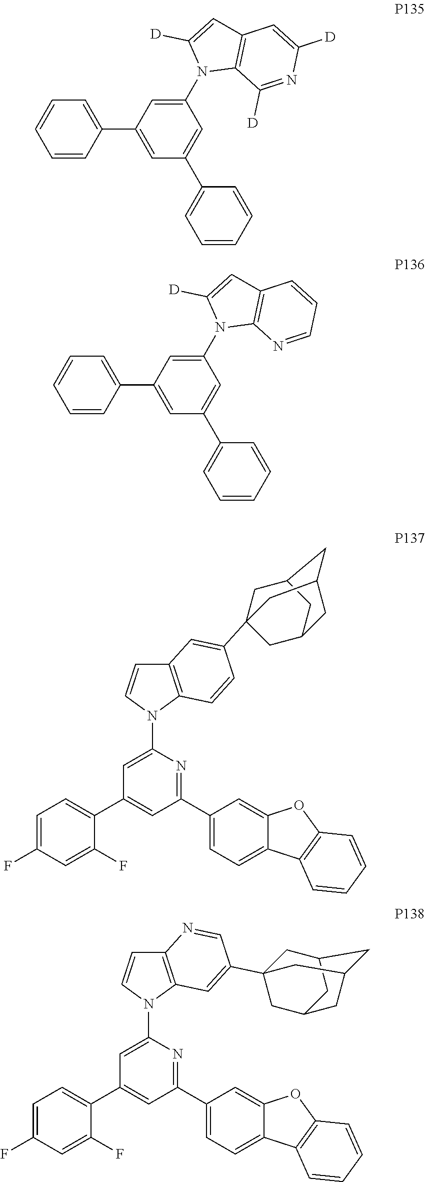

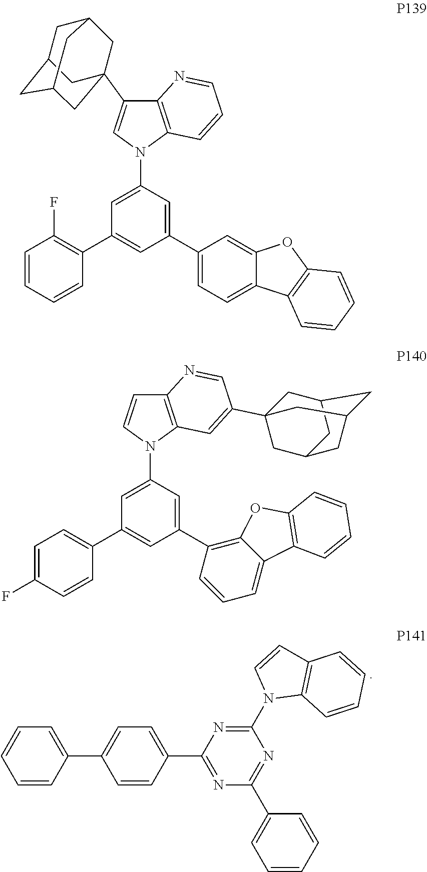 [SMILES: Fc1ccccc1-c1cc(-c2ccc3c(c2)oc2ccccc23)cc(-n2cc(C34CC5CC(CC(C5)C3)C4)c3ncccc32)c1 ; Fc1ccc(-c2cc(-c3cccc4c3oc3ccccc34)cc(-n3ccc4ncc(C56CC7CC(CC(C7)C5)C6)cc43)c2)cc1 ; c1ccc(-c2ccc(-c3nc(-c4ccccc4)nc(-n4ccc5ccccc54)n3)cc2)cc1]

In an embodiment, the present disclosure provides a light extraction material including any one of the organic compounds provided in the embodiments of the present disclosure.

In some optional embodiments of the present disclosure, a refractive index of the light extraction material in a visible light wavelength region is 1.5 to 1.7, which may be, for example, 1.51, 1.52, 1.54, 1.55, 1.56, 1.58, 1.6, 1.62, 1.64, 1.65, 1.66, 1.68 or the like.

In an embodiment, the present disclosure provides an organic electroluminescent element including an anode, a cathode and an organic layer disposed between the anode and the cathode, where a first capping layer is further disposed on a side of the cathode facing away from the anode and includes any one of the organic compounds provided in the embodiments of the present disclosure.

In some optional embodiments of the present disclosure, a second capping layer is further disposed between the first capping layer and the cathode, and a refractive index of a material of the second capping layer>a refractive index of the organic compound.

In some optional embodiments of the present disclosure, the refractive index of the material of the second capping layer>1.9, which may be, for example, 1.92, 1.95, 1.98, 2.0, 2.02, 2.05, 2.08, 2.10, 2.12, 2.15, 2.18, 2.2, 2.22, 2.25, 2.28, 2.3, 2.32, 2.35, 2.38 or the like.

In the present disclosure, the first capping layer formed by the organic compound having the structure represented by Formula I with a low refractive index is used in combination with the second capping layer with a high refractive index so that the light extraction effect of the element can be further optimized and the element has higher luminescence efficiency and lower color cast, thereby improving the accuracy of the color display and comprehensively improving the display effect.

In an embodiment, the present disclosure provides an organic electroluminescent element including an anode, a cathode and an organic layer disposed between the anode and the cathode, where the organic layer includes at least one of the organic compounds provided in the embodiments of the present disclosure.

In an embodiment, the organic layer includes a hole transport region, a light-emitting layer and an electron transport region that are disposed in sequence, where the hole transport region is located between the light-emitting layer and the anode, and the electron transport region is located between the light-emitting layer and the cathode.

In some optional embodiments of the present disclosure, the hole transport region includes any one or a combination of at least two of a hole injection layer, a hole transport layer and an electron blocking layer, and/or the electron transport region includes any one or a combination of at least two of an electron transport layer, an electron injection layer and a hole blocking layer.

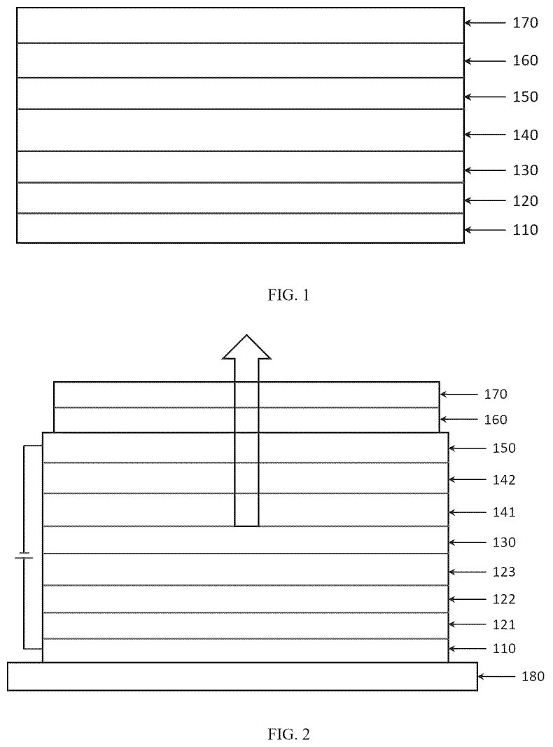

In some optional embodiments of the present disclosure, a structure diagram of the organic electroluminescent element is shown in FIG. 1. The organic electroluminescent element includes an anode 110, a hole transport region 120, a light-emitting layer 130, an electron transport region 140, a cathode 150, a second capping layer 160 and a first capping layer 170 that are disposed in sequence, where the first capping layer 170 includes any one of the organic compounds provided in the embodiments of the present disclosure, the hole transport region 120 includes any one or a combination of at least two of a hole injection layer (HIL), a hole transport layer (HTL) and an electron blocking layer (EBL), and the electron transport region 140 includes any one or a combination of at least two of an electron injection layer (EIL), an electron transport layer (ETL) and a hole blocking layer (HBL).

In some optional embodiments of the present disclosure, a substrate is further disposed on a side of the anode facing away from the hole transport region.

In some optional embodiments of the present disclosure, a structure diagram of the organic electroluminescent element is shown in FIG. 2. The organic electroluminescent element includes a substrate 180, an anode 110, a hole injection layer 121, a first hole transport layer 122, a second hole transport layer 123, a light-emitting layer 130, a first electron transport layer 141, a second electron transport layer 142, a cathode 150, a second capping layer 160 and a first capping layer 170 that are disposed in sequence, where the first capping layer 170 includes any one of the organic compounds provided in the embodiments of the present disclosure.

In some optional embodiments of the present disclosure, an anode material of the organic electroluminescent element may be a metal, a metal oxide or a conductive polymer. The metal includes copper, gold, silver, iron, chromium, nickel, manganese, palladium, platinum or the like and alloys thereof. The metal oxide includes indium tin oxide (ITO), indium zinc oxide (IZO), zinc oxide, indium gallium zinc oxide (IGZO) or the like. The conductive polymer includes polyaniline, polypyrrole, poly(3-methylthiophene) or the like. In addition to the above materials that facilitate hole injection and combinations thereof, the anode material further includes known materials suitable for use as the anode.

In some optional embodiments of the present disclosure, a cathode material of the organic electroluminescent element may be a metal or a multilayer metal material. The metal includes aluminum, magnesium, silver, indium, tin, titanium or the like and alloys thereof. The multilayer metal material includes LiF/Al, LiO₂/Al, BaF₂/Al or the like. In addition to the above materials that facilitate electron injection and combinations thereof, the cathode material further includes known materials suitable for use as the cathode.

The organic electroluminescent element may be prepared by the following method: forming the anode on a transparent or opaque smooth substrate, forming an organic layer on the anode, forming the cathode on the organic layer, and forming the first capping layer and the second capping layer on the cathode. The organic thin layer may be formed by a known film formation method such as evaporation, spin-coating, spin coating, impregnation and ion plating.

In an embodiment, the present disclosure provides a display device including any one of the organic electroluminescent elements provided in the embodiments of the present disclosure.

In a specific embodiment, the organic compound having the structure represented by Formula I in the present disclosure may be prepared according to the following synthesis route:

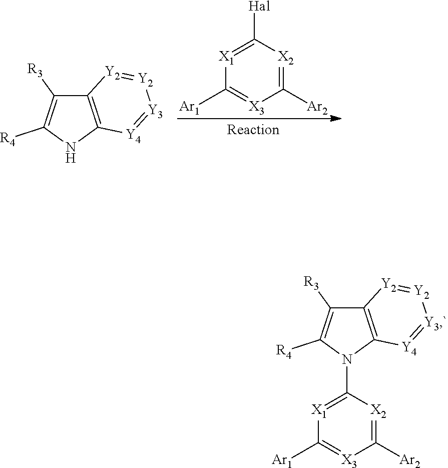

where Y₁, Y₂, Y₃, Y₄, X₁, X₂, X₃, R₃, R₄, Ar₁ and Ar₂ have the same definition as Formula I, and Hal is selected from any one of halogen, further preferably Br.

In a specific embodiment, a reaction is performed in the presence of a palladium catalyst, a ligand and an alkaline substance.

In a specific embodiment, the palladium catalyst is palladium (1-phenylallyl) chloride (Pd (cinnamyl) Cl)₂, and/or the ligand is

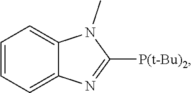

where t-Bu represents tert-butyl, and/or the alkaline substance is potassium t-butoxide KO(t-Bu).

The following compound embodiments exemplarily provide specific synthesis methods for a series of compounds. For compounds whose specific synthesis methods are not mentioned, these compounds may be synthesized by similar methods or other existing methods, which are not specifically limited in the present disclosure. In the following compound embodiments, the palladium catalysts used are (Pd (cinnamyl)Cl)₂, and the ligands used are

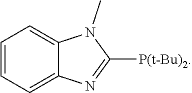

### Example 1

Organic Compound P4 having a structure of

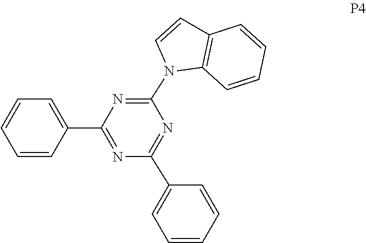 [SMILES: c1ccc(-c2nc(-c3ccccc3)nc(-n3ccc4ccccc43)n2)cc1]

was prepared by a method including the following steps:

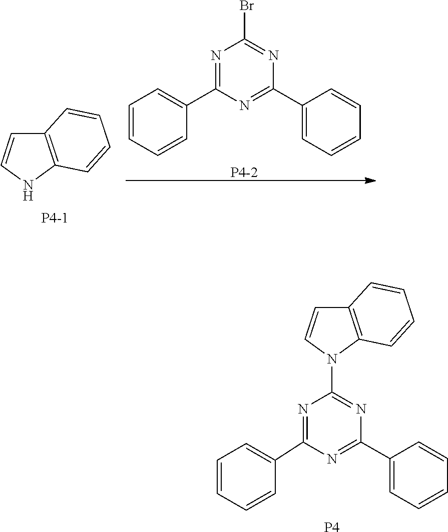

P4-1 (0.5 mmol), P4-2 (0.5 mmol), KO(t-Bu) (0.65 mmol), a palladium catalyst (1.0 mol %, based on an amount of P4-1 of 100 mol %) and a ligand (1.0 mol %, based on an amount of P4-1 of 100 mol %) were added to 3 mL toluene solution, mixed and placed in a 50 mL flask, and a reaction was performed for 12 h at 60° C. The solution was cooled to room temperature and slowly added with a saturated aqueous solution of MgSO₄ and ethyl acetate to be extracted three times. Then, the solvent was removed from the organic layer through a rotary evaporator, and the organic layer was subjected to column chromatography to obtain the product P4.

The structure of the target product P4 was tested through matrix-assisted laser desorption/ionization time-of-flight mass spectrometry (MALDI-TOF-MS) (m/z) to obtain C₂₃H₁₆N₄ whose calculated value was 348.4 and measured value was 348.1.

Elemental analysis (%): theoretical values: C 79.29, H 4.63, N 16.08; measured values: C 79.28, H 4.63, N 16.09.

### Example 2

Organic Compound P8 having a structure of

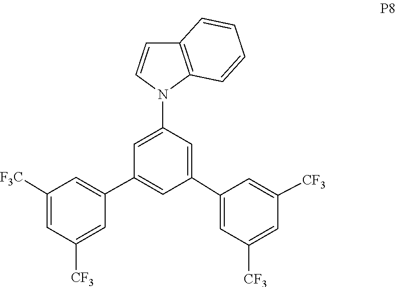 [SMILES: FC(F)(F)c1cc(-c2cc(-c3cc(C(F)(F)F)cc(C(F)(F)F)c3)cc(-n3ccc4ccccc43)c2)cc(C(F)(F)F)c1]

was prepared by a method including the following steps:

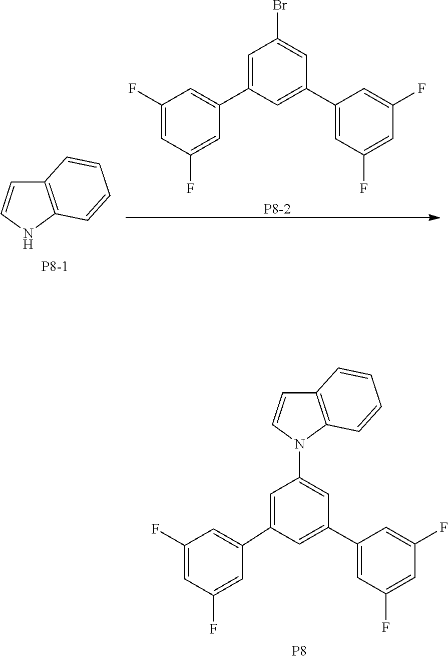

This example differs from Example 1 only in that P4-2 was replaced with an equimolar amount of P8-2, and other raw materials, amounts and process parameters were the same as those of Example 1 to obtain the target product P8.

The structure of the target product P8 was tested through matrix-assisted laser desorption/ionization time-of-flight mass spectrometry (MALDI-TOF-MS) (m/z) to obtain C₂₆H₁₅F₄N whose calculated value was 417.4 and measured value was 417.1.

Elemental analysis (%): theoretical values: C 74.82, H 3.62, N 3.36; measured values: C 74.80, H 3.63, N 3.37.

### Example 3

Organic Compound P9 having a structure of

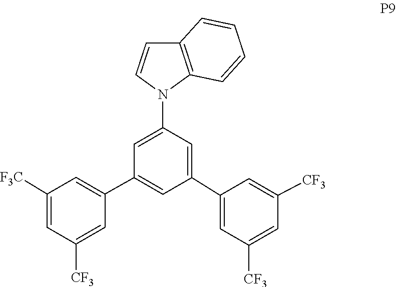 [SMILES: FC(F)(F)c1cc(-c2cc(-c3cc(C(F)(F)F)cc(C(F)(F)F)c3)cc(-n3ccc4ccccc43)c2)cc(C(F)(F)F)c1]

was prepared by a method including the following steps:

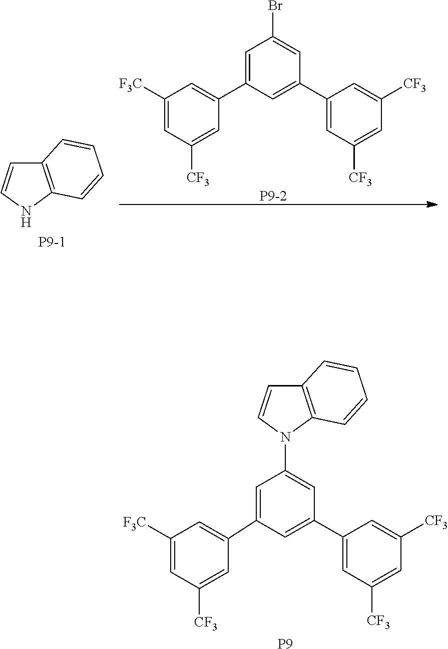

This example differs from Example 1 only in that P4-2 was replaced with an equimolar amount of P9-2, and other raw materials, amounts and process parameters were the same as those of Example 1 to obtain the target product P9.

The structure of the target product P9 was tested through matrix-assisted laser desorption/ionization time-of-flight mass spectrometry (MALDI-TOF-MS) (m/z) to obtain C₃₀H₁₅F₁₂N whose calculated value was 617.4 and measured value was 617.1.

Elemental analysis (%): theoretical values: C 58.36, H 2.45, N 2.27; measured values: C 58.36, H 2.47, N 2.25.

### Example 4

Organic Compound P64 having a structure of

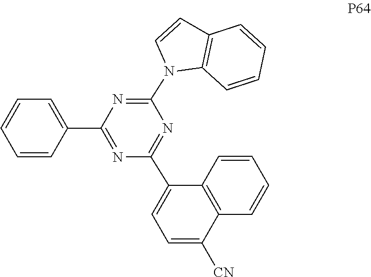 [SMILES: N#Cc1ccc(-c2nc(-c3ccccc3)nc(-n3ccc4ccccc43)n2)c2ccccc12]

was prepared by a method including the following steps:

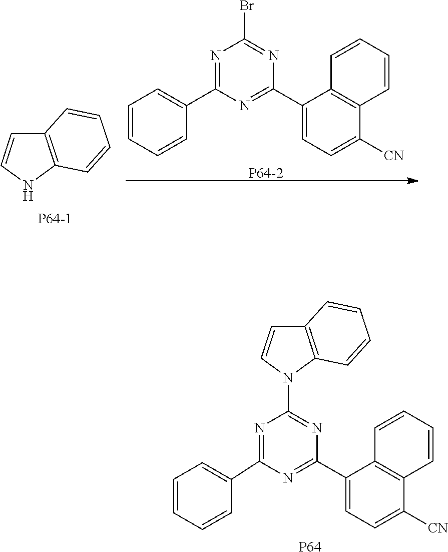

This example differs from Example 1 in that P4-2 was replaced with an equimolar amount of P64-2, and other raw materials and amounts were the same as those of Example 1. A reaction was performed for 12 h at 70° C. The solution was cooled to room temperature and slowly added with a saturated aqueous solution of MgSO₄ and ethyl acetate to be extracted three times. Then, the solvent was removed from the organic layer through a rotary evaporator and the organic layer was subjected to column chromatography to obtain the target product P64.

The structure of the target product P64 was tested through matrix-assisted laser desorption/ionization time-of-flight mass spectrometry (MALDI-TOF-MS) (m/z) to obtain C₂₈H₁₇N₅ whose calculated value was 423.5 and measured value was 423.1.

Elemental analysis (%): theoretical values: C 79.42, H 4.05, N 16.54; measured values: C 79.43, H 4.04, N 16.54.

### Example 5

Organic Compound P87 having a structure of

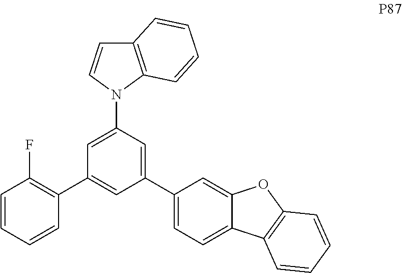 [SMILES: Fc1ccccc1-c1cc(-c2ccc3c(c2)oc2ccccc23)cc(-n2ccc3ccccc32)c1]

was prepared by a method including the following steps:

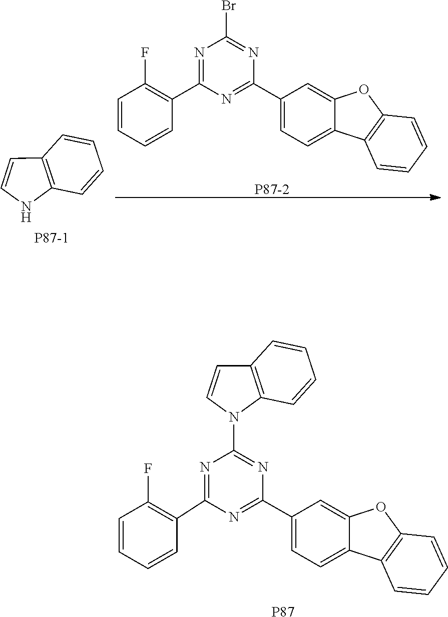

This example differs from Example 1 in that P4-2 was replaced with an equimolar amount of P87-2, and other raw materials and amounts were the same as those of Example 1. A reaction was performed for 12 h at 70° C. The solution was cooled to room temperature and slowly added with a saturated aqueous solution of MgSO₄ and ethyl acetate to be extracted three times. Then, the solvent was removed from the organic layer through a rotary evaporator, and the organic layer was subjected to column chromatography to obtain the target product P87.

The structure of the target product P87 was tested through matrix-assisted laser desorption/ionization time-of-flight mass spectrometry (MALDI-TOF-MS) (m/z) to obtain C₃₂H₂₀FNO whose calculated value was 453.5 and measured value was 453.2.

Elemental analysis (%): theoretical values: C 84.75, H 4.45, N 3.09; measured values: C 84.74, H 4.45, N 3.10.

The preparation methods for other organic compounds of the present disclosure are all similar to the preceding methods and not repeated herein. Only the characterization results of mass spectrometry and elemental analysis of some other compounds of the present disclosure are provided, which are shown in Table 1.

TABLE 1

| | Mass Spectrometry | | | |
|---|---|---|---|---|
| | Result | | | |
| Organic | Calculated | Measured | Elemental Analysis Result (%) | |
| Compound | value | Value | Theoretical Value | Measured Value |
| P15 | 615.7 | 615.2 | C, 74.13; H, 5.08; N, 2.28; | C, 74.13; H, 5.08; N, 2.29; |
| P16 | 529.7 | 529.3 | C, 86.17; H, 5.90; N, 7.93; | C, 86.17; H, 5.90; N, 7.92; |
| P24 | 618.4 | 618.1 | C, 56.32; H, 2.28; N, 4.53; | C, 56.32; H, 2.29; N, 4.53; |
| P35 | 482.4 | 482.1 | C, 67.22; H, 3.34; N, 5.81; | C, 67.22; H, 3.35; N, 5.81; |
| P39 | 619.4 | 619.1 | C, 54.29; H, 2.12; N, 6.78; | C, 54.29; H, 2.11; N, 6.78; |
| P79 | 606.8 | 606.2 | C, 81.15; H, 4.98; N, 9.23; | C, 81.15; H, 4.99; N, 9.23; |
| P80 | 451.6 | 451.2 | C, 90.43; H, 6.47; N, 3.10; | C, 90.43; H, 6.47; N, 3.11; |
| P124 | 523.7 | 523.2 | C, 82.57; H, 5.77; N, 8.02; | C, 82.57; H, 5.77; N, 8.01; |
| P135 | 343.4 | 343.1 | C, 87.44; H, 4.40; N, 8.16; | C, 87.44; H, 4.40; N, 8.17; |
| P139 | 588.7 | 588.3 | C, 83.65; H, 5.65; N, 4.76; | C, 83.65; H, 5.66; N, 4.76; |
| P141 | 424.5 | 424.2 | C, 82.05; H, 4.75; N, 13.20; | C, 82.05; H, 4.76; N, 13.20; |

### Performance Test Characterization of Refractive Indexes of Materials

The refractive indexes of the organic compounds at wavelengths of 460 nm, 530 nm and 620 nm were tested by an ellipsometer. A difference Δn₁ between the refractive index at a wavelength of 460 nm and the refractive index at a wavelength of 530 nm, a difference Δn₂ between the refractive index at a wavelength of 530 nm and the refractive index at a wavelength of 620 nm, and a difference Δn₃ between the refractive index at a wavelength of 460 nm and the refractive index at a wavelength of 620 nm were calculated. The test results are shown in Table 2.

TABLE 2

| Organic | | | | | | |
|---|---|---|---|---|---|---|
| Compound | n_{460 nm} | n_{530 nm} | n_{620 nm} | Δn₁ | Δn₂ | Δn₃ |
| P4 | 1.70 | 1.65 | 1.63 | 0.05 | 0.02 | 0.07 |
| P8 | 1.66 | 1.62 | 1.60 | 0.04 | 0.02 | 0.06 |
| P9 | 1.58 | 1.55 | 1.54 | 0.03 | 0.01 | 0.04 |
| P15 | 1.63 | 1.59 | 1.57 | 0.04 | 0.02 | 0.06 |
| P16 | 1.67 | 1.63 | 1.61 | 0.04 | 0.02 | 0.06 |
| P24 | 1.57 | 1.54 | 1.53 | 0.03 | 0.01 | 0.04 |
| P35 | 1.63 | 1.59 | 1.57 | 0.04 | 0.02 | 0.06 |
| P39 | 1.55 | 1.53 | 1.52 | 0.02 | 0.01 | 0.03 |
| P64 | 1.70 | 1.65 | 1.63 | 0.05 | 0.02 | 0.07 |
| P79 | 1.68 | 1.64 | 1.63 | 0.04 | 0.01 | 0.05 |
| P80 | 1.68 | 1.64 | 1.61 | 0.04 | 0.03 | 0.07 |
| P87 | 1.69 | 1.65 | 1.62 | 0.04 | 0.03 | 0.07 |
| P124 | 1.68 | 1.64 | 1.61 | 0.04 | 0.03 | 0.07 |
| P135 | 1.67 | 1.63 | 1.61 | 0.04 | 0.02 | 0.06 |
| P139 | 1.65 | 1.61 | 1.59 | 0.04 | 0.02 | 0.06 |
| P141 | 1.75 | 1.70 | 1.67 | 0.05 | 0.03 | 0.08 |
| C1 | 2.09 | 1.99 | 1.90 | 0.10 | 0.09 | 0.19 |
| C2 | 2.21 | 2.08 | 1.99 | 0.13 | 0.09 | 0.22 |
| C3 | 2.18 | 2.05 | 1.95 | 0.13 | 0.10 | 0.23 |
| C4 | 2.22 | 2.08 | 1.98 | 0.14 | 0.10 | 0.24 |
| C5 | 2.00 | 1.90 | 1.82 | 0.10 | 0.08 | 0.18 |

In Table 2, structures of C1 to C5 are as follows:

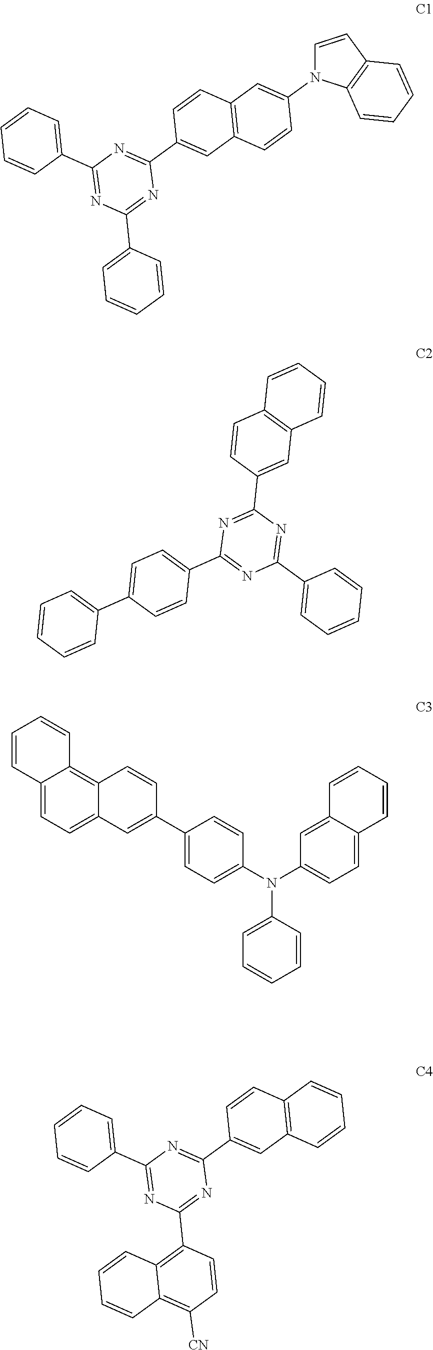 [SMILES: c1ccc(-c2nc(-c3ccccc3)nc(-c3ccc4cc(-n5ccc6ccccc65)ccc4c3)n2)cc1 ; c1ccc(-c2ccc(-c3nc(-c4ccccc4)nc(-c4ccc5ccccc5c4)n3)cc2)cc1 ; c1ccc(N(c2ccc(-c3ccc4c(ccc5ccccc54)c3)cc2)c2ccc3ccccc3c2)cc1 ; N#Cc1ccc(-c2nc(-c3ccccc3)nc(-c3ccc4ccccc4c3)n2)c2ccccc12]

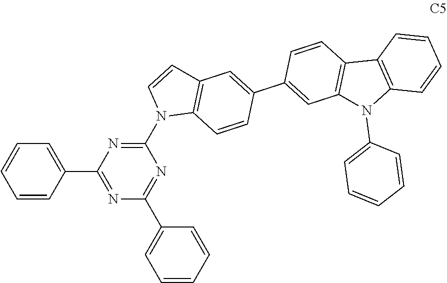 [SMILES: c1ccc(-c2nc(-c3ccccc3)nc(-n3ccc4cc(-c5ccc6c7ccccc7n(-c7ccccc7)c6c5)ccc43)n2)cc1]

As can be seen from Table 2, the refractive indexes of the organic compounds provided in the present disclosure at wavelengths of 460 nm, 530 nm and 620 nm are 1.5 to 1.7, the difference between the refractive index at a wavelength of 460 nm and the refractive index at a wavelength of 530 nm is 0.02 to 0.05, the difference between the refractive index at a wavelength of 530 nm and the refractive index at a wavelength of 620 nm is 0.01 to 0.03, and the difference between the refractive index at a wavelength of 460 nm and the refractive index at a wavelength of 620 nm is 0.04 to 0.08. When the organic compounds provided in the present disclosure are used for organic electroluminescent elements, since the difference Δn between the refractive indexes at different wavelengths is relatively small, a difference between refractive indexes in different light colors is small, and when display is performed at multiple angles, the color cast can be effectively improved.

Several application examples of the organic compounds of the present disclosure applied to organic electroluminescent elements (OLED elements) are listed below.

### Application Example 1

A structure diagram of an organic electroluminescent element is shown in FIG. 2. A substrate 180, an anode 110 (indium tin oxide, ITO), a hole injection layer 121, a first hole transport layer 122, a second hole transport layer 123, a light-emitting layer 130, a first electron transport layer 141, a second electron transport layer 142, a cathode 150 (a magnesium-silver electrode with a mass ratio of magnesium to silver of 9:1), a second capping layer 160 and a first capping layer 170 are stacked in sequence.

A method for preparing the organic electroluminescent element includes the steps described below.
(1) A glass substrate having an ITO anode (with a thickness of 15 nm) was cut into a size of 50 mm×50 mm×0.7 mm, sonicated in isopropyl alcohol and deionized water for 30 min separately, and cleaned under ozone for about 10 min. The cleaned anode substrate was installed onto a vacuum deposition apparatus.
(2) A material of the hole injection layer (Compound a) and a p-type doping material (Compound b) were deposited through vacuum evaporation on the ITO anode layer at a doping ratio (mass ratio) of 3% for use as the hole injection layer with a thickness of 5 nm.
(3) A material of the hole transport layer (Compound b) was deposited through vacuum evaporation on the hole injection layer for use as the first hole transport layer with a thickness of 100 nm.
(4) A hole transport material (Compound c) was deposited through vacuum evaporation on the first hole transport layer for use as the second hole transport layer with a thickness of 5 nm.
(5) The light-emitting layer with a thickness of 30 nm was deposited through vacuum evaporation on the second hole transport layer, where Compound d was doped as a host material with Compound e as a doping material at a doping ratio (mass ratio) of 3%.
(6) An electron transport material (Compound f) was deposited through vacuum evaporation on the light-emitting layer for use as the first electron transport layer with a thickness of 30 nm.
(7) An electron transport material (Compound g) and an n-type doping material (Compound h) were co-deposited through vacuum evaporation on the first electron transport layer at a doping ratio (mass ratio) of 1:1 for use as the second electron transport layer with a thickness of 5 nm.
(8) The magnesium-silver electrode (Mg:Ag was 9:1) was deposited through vacuum evaporation on the second electron transport layer for use as the cathode with a thickness of 10 nm.
(9) Compound i was deposited through vacuum evaporation on the cathode for use as the second capping layer with a thickness of 100 nm.
(10) Small Organic Molecular Compound P4 provided in the present disclosure was deposited through vacuum evaporation on the second capping layer for use as the first capping layer with a thickness of 20 nm to obtain the organic electroluminescent element.

Structures of the materials used in the organic electroluminescent element are as follows:

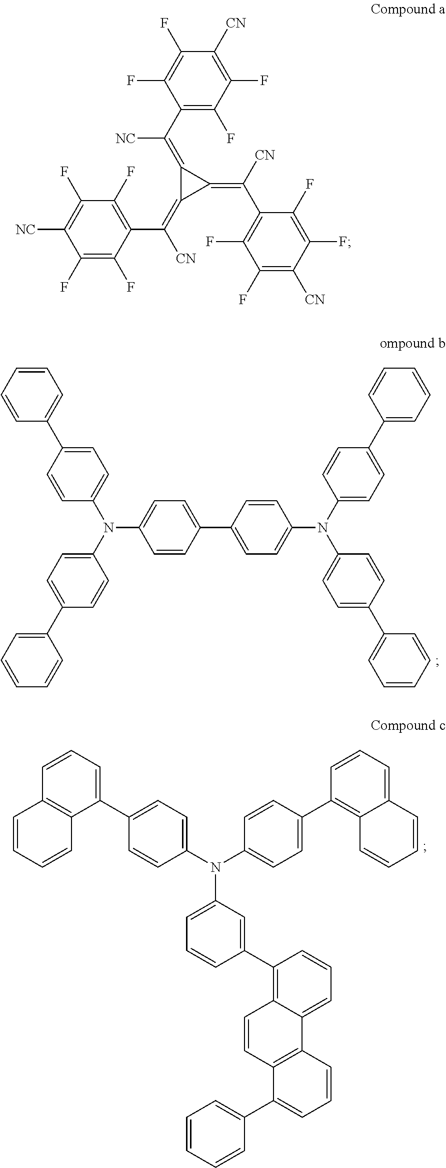 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2cccc3c2ccc2c(-c4cccc(N(c5ccc(-c6cccc7ccccc67)cc5)c5ccc(-c6cccc7ccccc67)cc5)c4)cccc23)cc1]

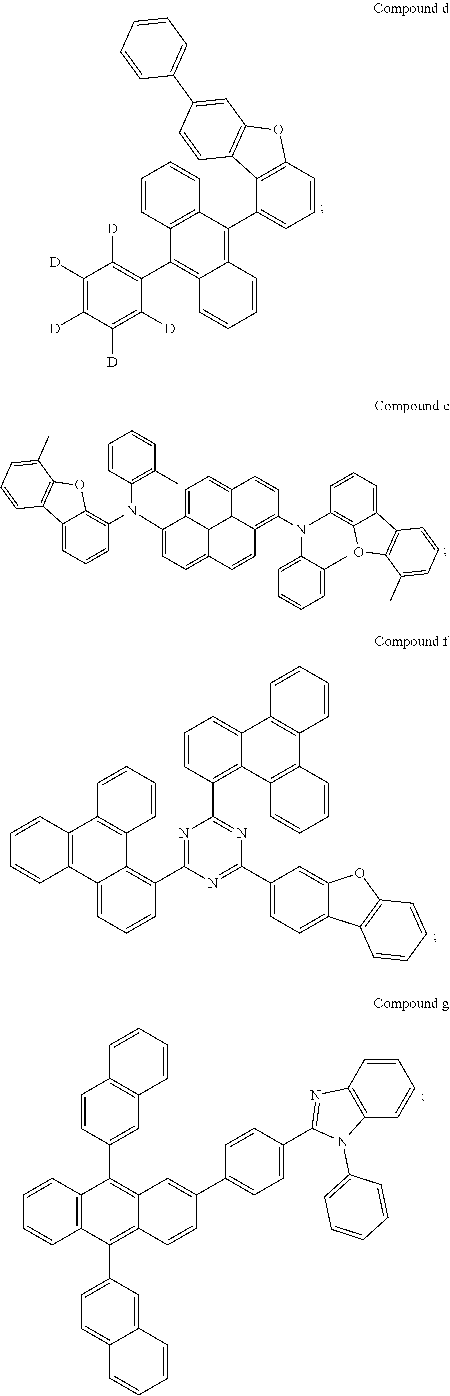 [SMILES: [2H]c1c([2H])c([2H])c(-c2c3ccccc3c(-c3cccc4oc5cc(-c6ccccc6)ccc5c34)c3ccccc23)c([2H])c1[2H] ; Cc1ccccc1N(C1=CC=C2C=CC3=C(N(c4ccccc4C)c4cccc5c4oc4c(C)cccc45)C=CC4=CC=C1C2C43)c1cccc2c1oc1c(C)cccc12 ; c1ccc2c(c1)oc1cc(-c3nc(-c4cccc5c6ccccc6c6ccccc6c45)nc(-c4cccc5c6ccccc6c6ccccc6c45)n3)ccc12 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

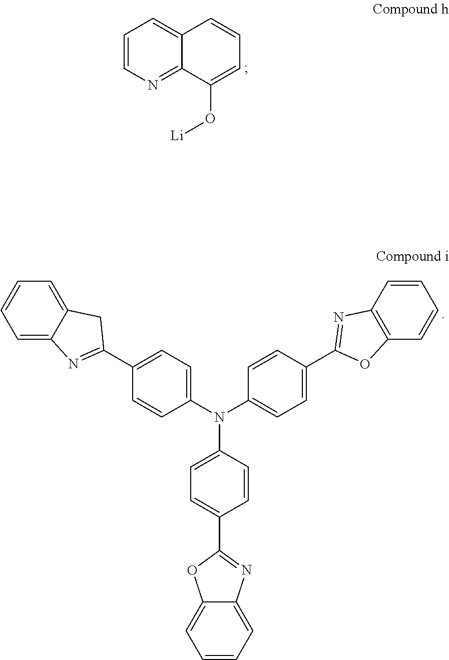

### Application Examples 2 to 16 and Comparative Examples 1 to 5

An organic electroluminescent element differs from Application Example 1 only in that the material of the first capping layer in step (10) was replaced with a respective compound shown in Table 3, and the structure of the element, thicknesses, other materials and the preparation method were the same as those of Application Example 1.

The OLED element was subjected to the performance test described below.

A Keithley 2365A digital nanovoltmeter was used for testing currents of the OLED element at different voltages, and then the currents were divided by a luminescence area to obtain current densities of the OLED element at different voltages. A Konicaminolta CS-2000 spectroradiometer was used for testing the brightness and radiation energy flux densities of the OLED element at different voltages. According to the current densities and brightness of the organic electroluminescent element at different voltages, an operating voltage Von (V), current efficiency CE (cd/A) and multi-angle color cast JNCD) (30°/45°/60° at the same current density (10 mA/cm²) were obtained. The results are shown in Table 3.

TABLE 3

| | First Capping | Vₒₙ | CE | JNCD |
|---|---|---|---|---|
| Element No. | Layer | (V) | (cd/A) | (30°/45°/60°) |
| Application | P4 | 3.48 | 8.28 | 4/2/1 |
| Example 1 | | | | |
| Application | P8 | 3.50 | 8.32 | 2/2/2 |
| Example 2 | | | | |
| Application | P9 | 3.50 | 8.35 | 3/2/1 |
| Example 3 | | | | |
| Application | P15 | 3.49 | 8.30 | 3/2/1 |
| Example 4 | | | | |
| Application | P16 | 3.48 | 8.32 | 2/2/1 |
| Example 5 | | | | |
| Application | P24 | 3.51 | 8.36 | 2/2/1 |
| Example 6 | | | | |
| Application | P35 | 3.50 | 8.31 | 3/3/1 |
| Example 7 | | | | |
| Application | P39 | 3.49 | 8.40 | 2/1/1 |
| Example 8 | | | | |
| Application | P64 | 3.50 | 8.32 | 3/2/1 |
| Example 9 | | | | |
| Application | P79 | 3.50 | 8.33 | 2/1/2 |
| Example 10 | | | | |
| Application | P80 | 3.50 | 8.30 | 3/2/2 |
| Example 11 | | | | |
| Application | P87 | 3.50 | 8.36 | 3/1/2 |
| Example 12 | | | | |
| Application | P124 | 3.50 | 8.38 | 3/3/1 |
| Example 13 | | | | |
| Application | P135 | 3.49 | 8.27 | 3/3/1 |
| Example 14 | | | | |
| Application | P139 | 3.49 | 8.33 | 3/3/2 |
| Example 15 | | | | |
| Application | P141 | 3.50 | 8.24 | 4/2/1 |
| Example 16 | | | | |
| Comparative | C1 | 3.50 | 7.13 | 6/4/3 |
| Example 1 | | | | |
| Comparative | C2 | 3.50 | 6.83 | 7/6/5 |
| Example 2 | | | | |
| Comparative | C3 | 3.50 | 6.89 | 6/4/2 |
| Example 3 | | | | |
| Comparative | C4 | 3.50 | 6.82 | 7/5/6 |
| Example 4 | | | | |
| Comparative | C5 | 3.49 | 7.34 | 6/3/3 |
| Example 5 | | | | |

As can be seen from Table 3, the voltage of the element using the organic compound provided in the present disclosure as the first capping layer in combination with the second capping layer containing a small organic molecule with a high refractive index is basically equivalent to that of the element using Compounds C1 to C5 as the first capping layer in combination with the second capping layer. The organic compound of the present disclosure can more significantly improve the luminescence efficiency of the element and, in particular, has a very significant advantage in improving the color cast so that the element has more prominent accuracy of color display.

The applicant states that although the organic compound and the application thereof of the present disclosure are described through the preceding examples, the present disclosure is not limited to the preceding process steps, which means that the implementation of the present disclosure does not necessarily depend on the preceding process steps. Those skilled in the art are to understand that any improvements made to the present disclosure, equivalent substitutions of selected raw materials, additions of adjuvant ingredients, selections of specific manners or the like in the present disclosure all fall within the protection scope and the disclosure scope of the present disclosure.

What is claimed is:

1. An organic compound, having a structure represented by Formula I: 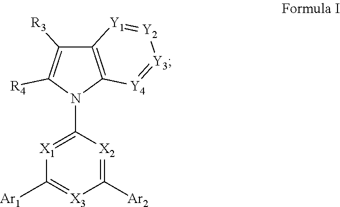
wherein Y₁, Y₂, Y₃ and Y₄ are each independently selected from CR₁ or N;
X₁, X₂ and X₃ are each independently selected from CR₂ or N;
R₁, R₂, R₃ and R₄ are each independently selected from any one of hydrogen, deuterium, halogen, cyano, isocyano, substituted or unsubstituted C1 to C20 linear or branched alkyl, substituted or unsubstituted C1 to C20 alkoxy, substituted or unsubstituted C1 to C20 alkylthio, substituted or unsubstituted C3 to C20 cycloalkyl, substituted or unsubstituted C6 to C30 aryl or substituted or unsubstituted C6 to C30 arylsilyl;
Ar₁ and Ar₂ are each independently selected from any one of halogen, cyano, isocyano, substituted or unsubstituted C1 to C20 linear or branched alkyl, substituted or unsubstituted C1 to C20 alkoxy, substituted or unsubstituted C1 to C20 alkylthio, substituted or unsubstituted C3 to C20 cycloalkyl, substituted or unsubstituted C6 to C30 aryl, substituted or unsubstituted C3 to C30 heteroaryl, substituted or unsubstituted C6 to C30 ketoaryl, substituted or unsubstituted C3 to C30 ketoheteroaryl, substituted or unsubstituted C6 to C30 aryl sulfonyl, substituted or unsubstituted C3 to C30 heteroaryl sulfonyl, substituted or unsubstituted C6 to C30 aryl phosphorus oxygen or substituted or unsubstituted C3 to C30 heteroaryl phosphorus oxygen; and substituents for substitutions in R₁, R₂, R₃, R₄, Ar₁ and Ar₂ are each independently selected from at least one of deuterium, halogen, cyano, isocyano, unsubstituted or halogenated C1 to C12 linear or branched alkyl, C3 to C12 cycloalkyl, unsubstituted or halogenated C1 to C12 alkoxy, unsubstituted or halogenated C1 to C12 alkylthio, C6 to C20 aryl, C3 to C20 heteroaryl, C6 to C20 aryl ester or C6 to C20 arylsilyl.

2. The organic compound according to claim 1, wherein Y₁, Y₂, Y₃ and Y₄ are each independently selected from CR₁ or N, and a number of N is less than or equal to 2.

3. The organic compound according to claim 1, wherein the organic compound has a structure represented by any one of Formula II-1, Formula II-2, Formula II-3, Formula II-4 or Formula II-5: 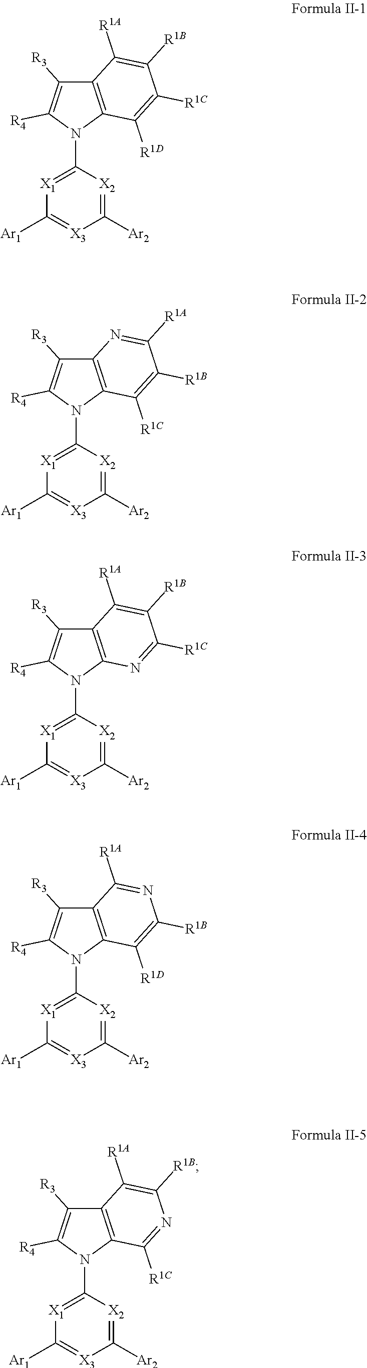
wherein X₁, X₂, X₃, R₃, R₄, Ar₁ and Ar₂ have a same defined range as Formula I;
R^{1A}, R^{1B}, R^{1C} and R^{1D} are each independently selected from any one of hydrogen, deuterium, halogen, cyano, isocyano, substituted or unsubstituted C1 to C20 linear or branched alkyl, substituted or unsubstituted C1 to C20 alkoxy, substituted or unsubstituted C1 to C20 alkylthio, substituted or unsubstituted C3 to C20 cycloalkyl, substituted or unsubstituted C6 to C30 aryl, substituted or unsubstituted C3 to C30 heteroaryl or substituted or unsubstituted C6 to C30 arylsilyl; and
Substituents for substitutions in R^{1A}, R^{1B}, R^{1C} and R^{1D} are each independently selected from at least one of deuterium, halogen, cyano, isocyano, unsubstituted or halogenated C1 to C12 linear or branched alkyl, C3 to C12 cycloalkyl, unsubstituted or halogenated C1 to C12 alkoxy, unsubstituted or halogenated C1 to C12 alkylthio, C6 to C20 aryl, C3 to C20 heteroaryl, C6 to C20 aryl ester or C6 to C20 arylsilyl.

4. The organic compound according to claim 1, wherein X₁, X₂ and X₃ are each independently selected from CH or N.

5. The organic compound according to claim 1, wherein R₁, R₃ and R₄ are each independently selected from any one of hydrogen, deuterium, halogen, cyano, unsubstituted or halogenated C1 to C12 linear or branched alkyl, C3 to C12 cycloalkyl, unsubstituted or halogenated C1 to C12 alkoxy or unsubstituted or halogenated C1 to C12 alkylthio.

6. The organic compound according to claim 3, wherein R^{1A}, R^{1B}, R^{1C}, R^{1D}, R₃ and R₄ are each independently selected from any one of hydrogen, deuterium, halogen, cyano, unsubstituted or halogenated C1 to C12 linear or branched alkyl, C3 to C12 cycloalkyl, unsubstituted or halogenated C1 to C12 alkoxy or unsubstituted or halogenated C1 to C12 alkylthio.

7. The organic compound according to claim 1, wherein Ar₁ and Ar₂ are each independently selected from any one of substituted or unsubstituted C6 to C30 aryl, substituted or unsubstituted C3 to C30 heteroaryl, substituted or unsubstituted C6 to C30 ketoaryl, substituted or unsubstituted C3 to C30 ketoheteroaryl, substituted or unsubstituted C6 to C30 aryl sulfonyl or substituted or unsubstituted C6 to C30 aryl phosphorus oxygen; and
substituents for substitutions are each independently selected from at least one of deuterium, halogen, cyano, isocyano, unsubstituted or halogenated C1 to C12 linear or branched alkyl, C3 to C12 cycloalkyl, unsubstituted or halogenated C1 to C12 alkoxy, unsubstituted or halogenated C1 to C12 alkylthio, C6 to C20 aryl, C3 to C20 heteroaryl, C6 to C20 aryl ester or C6 to C20 arylsilyl.

8. The organic compound according to claim 1, wherein Ar₁ and Ar₂ are each independently selected from any one of the following substituted or unsubstituted groups: 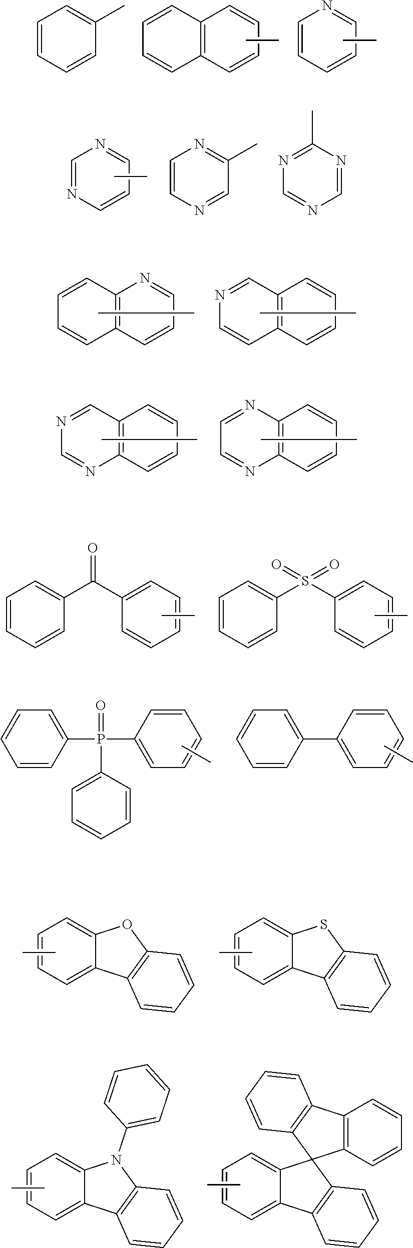 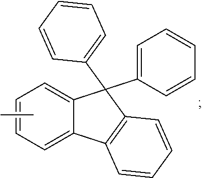
wherein the dashed line represents a linkage site of the group; and
substituents for substitutions in Ar₁ and Ar₂ are each independently selected from at least one of deuterium, halogen, cyano, isocyano, unsubstituted or halogenated C1 to C12 linear or branched alkyl, C3 to C12 cycloalkyl, unsubstituted or halogenated C1 to C12 alkoxy, unsubstituted or halogenated C1 to C12 alkylthio, C6 to C20 aryl, C6 to C20 aryl ester or C6 to C20 arylsilyl.

9. The organic compound according to claim 8, wherein the substituents for the substitutions in Ar₁ and Ar₂ are each independently selected from at least one of deuterium, fluorine, cyano, isocyano, unsubstituted or fluorinated C1 to C6 linear or branched alkyl, cyclohexyl, adamantyl, unsubstituted or fluorinated C1 to C6 alkoxy, phenyl, naphthyl, phenyl ester or triphenylsilyl.

10. The organic compound according to claim 1, wherein at least one of Ar₁ or Ar₂ comprises an electron withdrawing group.

11. The organic compound according to claim 1, wherein at least one of Ar₁ or Ar₂ is selected from any one of the following groups: 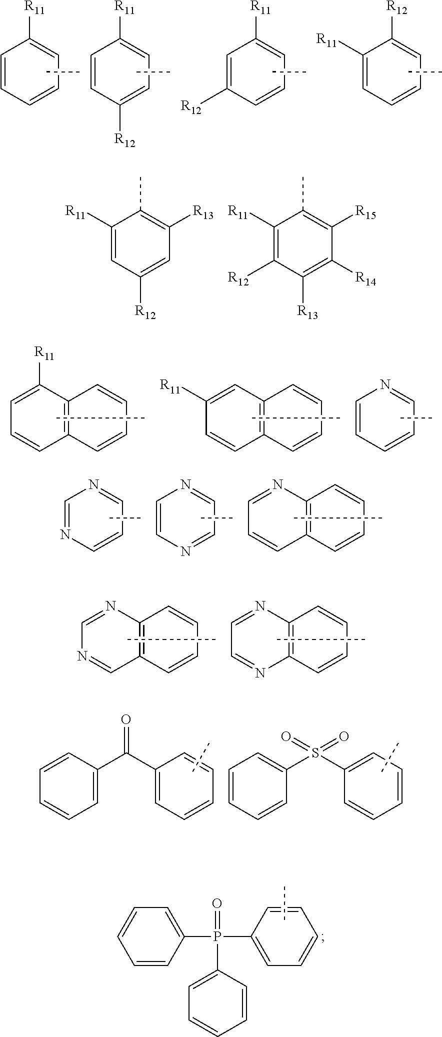
wherein the dashed line represents a linkage site of the group; and
R₁₁, R₁₂, R₁₃, R₁₄ and R₁₅ are each independently selected from any one of fluorine, cyano, isocyano, fluorinated C1 to C6 linear or branched alkyl or fluorinated C1 to C6 alkoxy.

12. The organic compound according to claim 1, wherein one of Ar₁ or Ar₂ is selected from any one of the following groups: 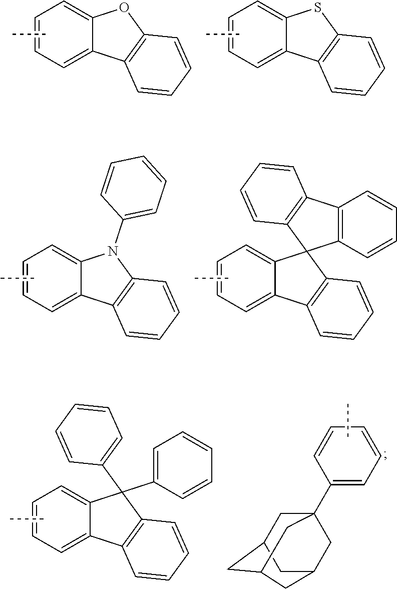
wherein the dashed line represents a linkage site of the group.

13. The organic compound according to claim 1, wherein the organic compound is selected from any one of the following compounds P1 to P141:

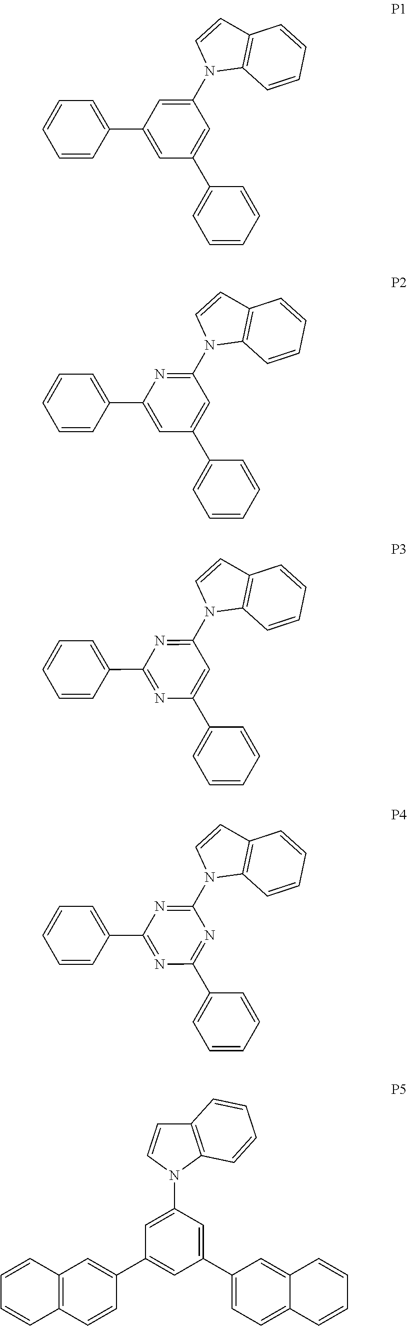

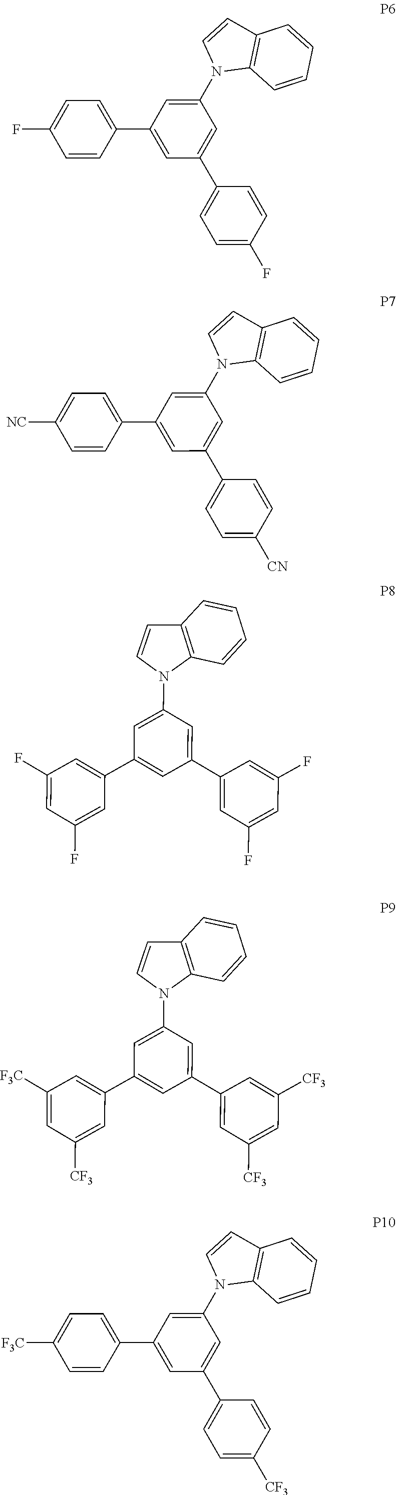 [SMILES: Fc1ccc(-c2cc(-c3ccc(F)cc3)cc(-n3ccc4ccccc43)c2)cc1 ; N#Cc1ccc(-c2cc(-c3ccc(C#N)cc3)cc(-n3ccc4ccccc43)c2)cc1 ; Fc1cc(F)cc(-c2cc(-c3cc(F)cc(F)c3)cc(-n3ccc4ccccc43)c2)c1 ; FC(F)(F)c1cc(-c2cc(-c3cc(C(F)(F)F)cc(C(F)(F)F)c3)cc(-n3ccc4ccccc43)c2)cc(C(F)(F)F)c1 ; FC(F)(F)c1ccc(-c2cc(-c3ccc(C(F)(F)F)cc3)cc(-n3ccc4ccccc43)c2)cc1]

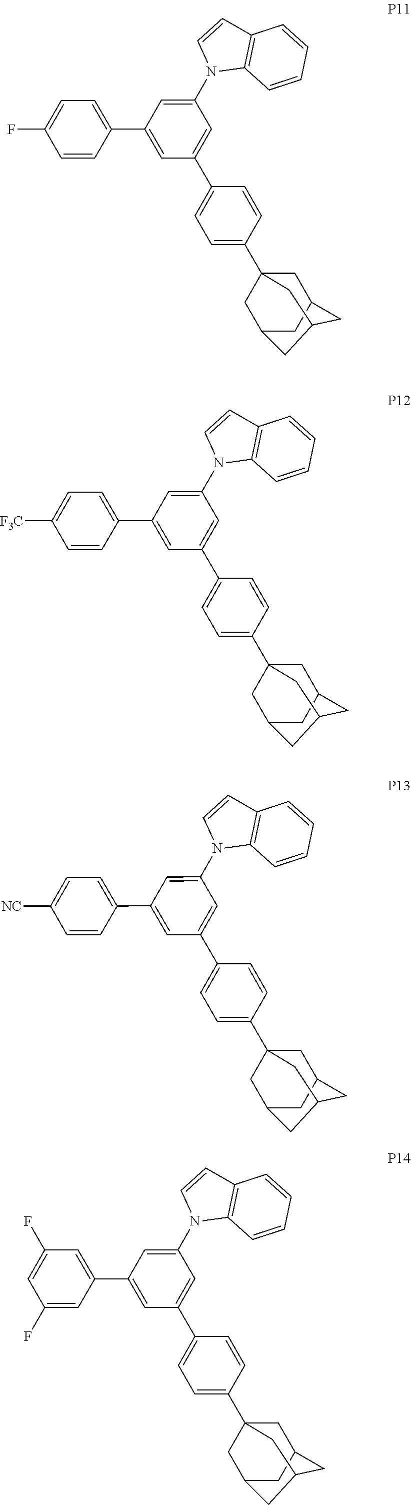 [SMILES: Fc1ccc(-c2cc(-c3ccc(C45CC6CC(CC(C6)C4)C5)cc3)cc(-n3ccc4ccccc43)c2)cc1 ; FC(F)(F)c1ccc(-c2cc(-c3ccc(C45CC6CC(CC(C6)C4)C5)cc3)cc(-n3ccc4ccccc43)c2)cc1 ; N#Cc1ccc(-c2cc(-c3ccc(C45CC6CC(CC(C6)C4)C5)cc3)cc(-n3ccc4ccccc43)c2)cc1 ; Fc1cc(F)cc(-c2cc(-c3ccc(C45CC6CC(CC(C6)C4)C5)cc3)cc(-n3ccc4ccccc43)c2)c1]

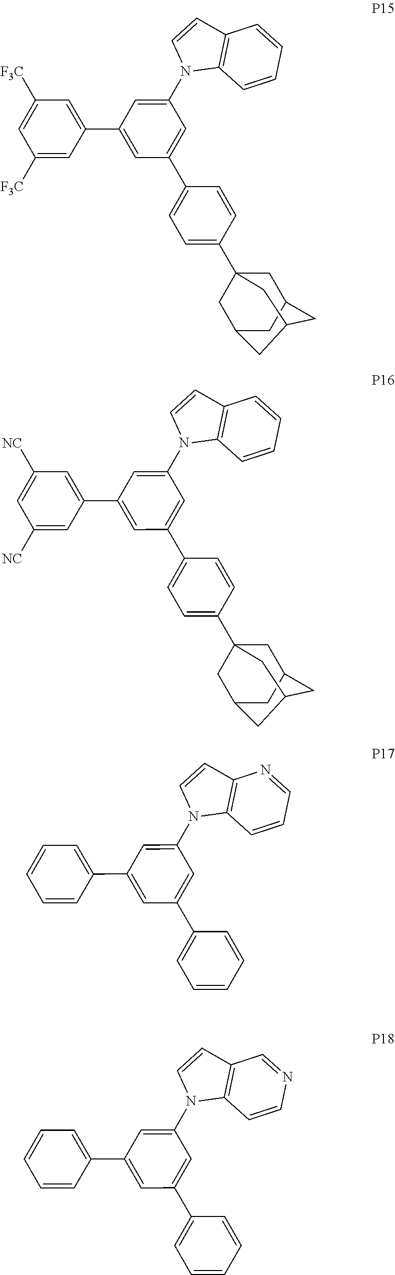

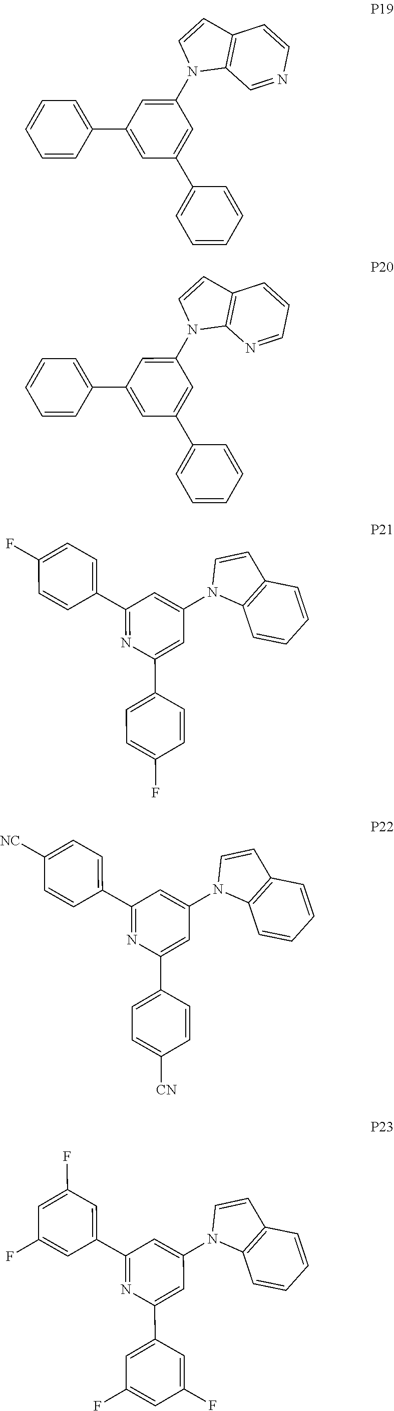

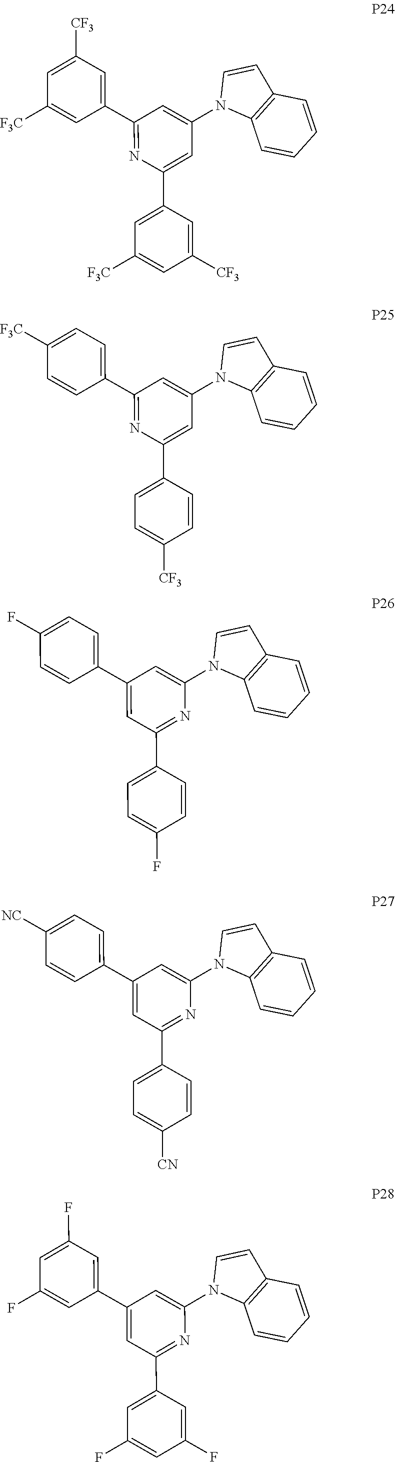 [SMILES: FC(F)(F)c1cc(-c2cc(-n3ccc4ccccc43)cc(-c3cc(C(F)(F)F)cc(C(F)(F)F)c3)n2)cc(C(F)(F)F)c1 ; FC(F)(F)c1ccc(-c2cc(-n3ccc4ccccc43)cc(-c3ccc(C(F)(F)F)cc3)n2)cc1 ; Fc1ccc(-c2cc(-c3ccc(F)cc3)nc(-n3ccc4ccccc43)c2)cc1 ; N#Cc1ccc(-c2cc(-c3ccc(C#N)cc3)nc(-n3ccc4ccccc43)c2)cc1 ; Fc1cc(F)cc(-c2cc(-c3cc(F)cc(F)c3)nc(-n3ccc4ccccc43)c2)c1]

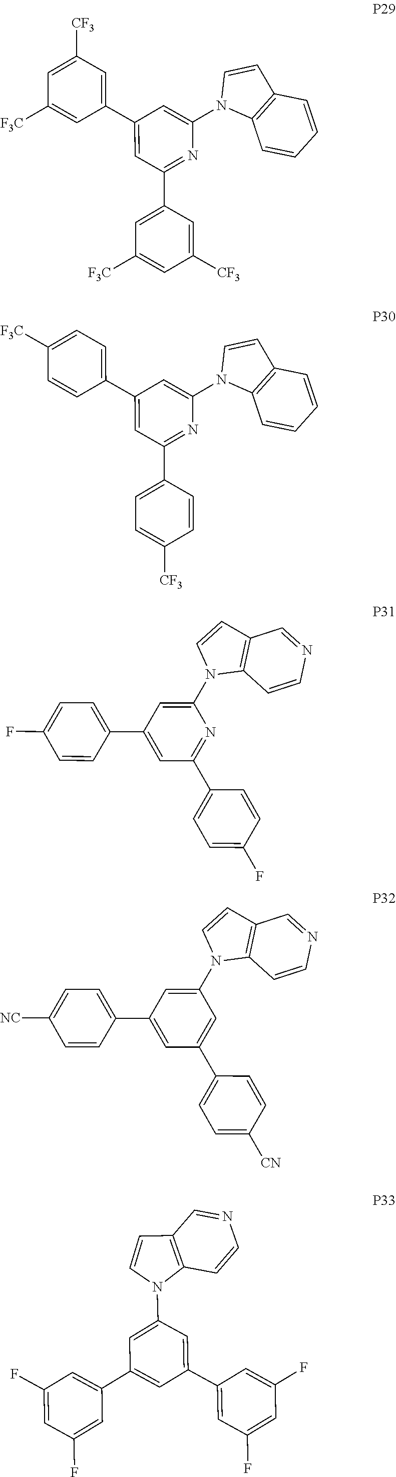

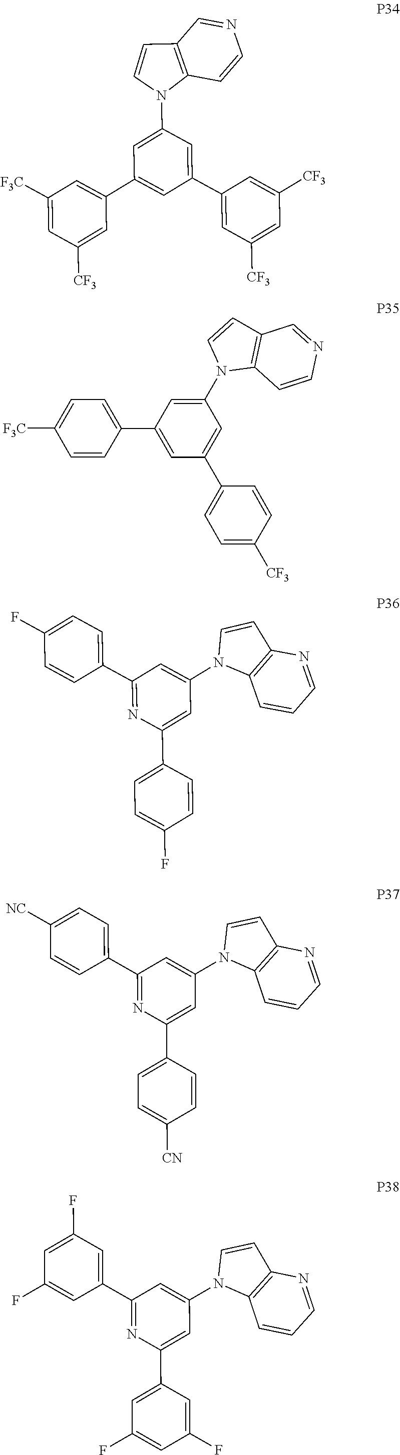

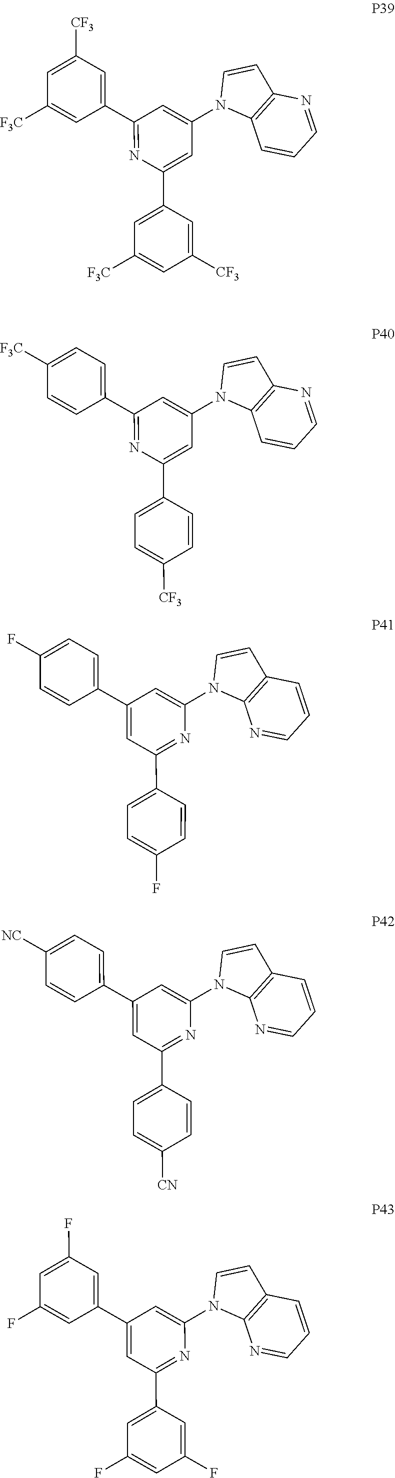 [SMILES: FC(F)(F)c1cc(-c2cc(-n3ccc4ncccc43)cc(-c3cc(C(F)(F)F)cc(C(F)(F)F)c3)n2)cc(C(F)(F)F)c1 ; FC(F)(F)c1ccc(-c2cc(-n3ccc4ncccc43)cc(-c3ccc(C(F)(F)F)cc3)n2)cc1 ; Fc1ccc(-c2cc(-c3ccc(F)cc3)nc(-n3ccc4cccnc43)c2)cc1 ; N#Cc1ccc(-c2cc(-c3ccc(C#N)cc3)nc(-n3ccc4cccnc43)c2)cc1 ; Fc1cc(F)cc(-c2cc(-c3cc(F)cc(F)c3)nc(-n3ccc4cccnc43)c2)c1]

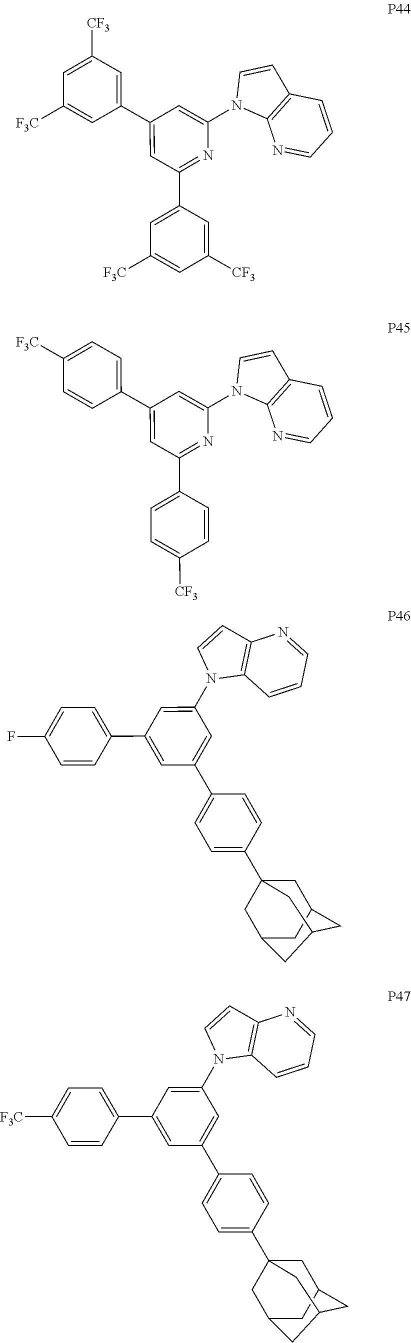 [SMILES: FC(F)(F)c1cc(-c2cc(-c3cc(C(F)(F)F)cc(C(F)(F)F)c3)nc(-n3ccc4cccnc43)c2)cc(C(F)(F)F)c1 ; FC(F)(F)c1ccc(-c2cc(-c3ccc(C(F)(F)F)cc3)nc(-n3ccc4cccnc43)c2)cc1 ; Fc1ccc(-c2cc(-c3ccc(C45CC6CC(CC(C6)C4)C5)cc3)cc(-n3ccc4ncccc43)c2)cc1 ; FC(F)(F)c1ccc(-c2cc(-c3ccc(C45CC6CC(CC(C6)C4)C5)cc3)cc(-n3ccc4ncccc43)c2)cc1]

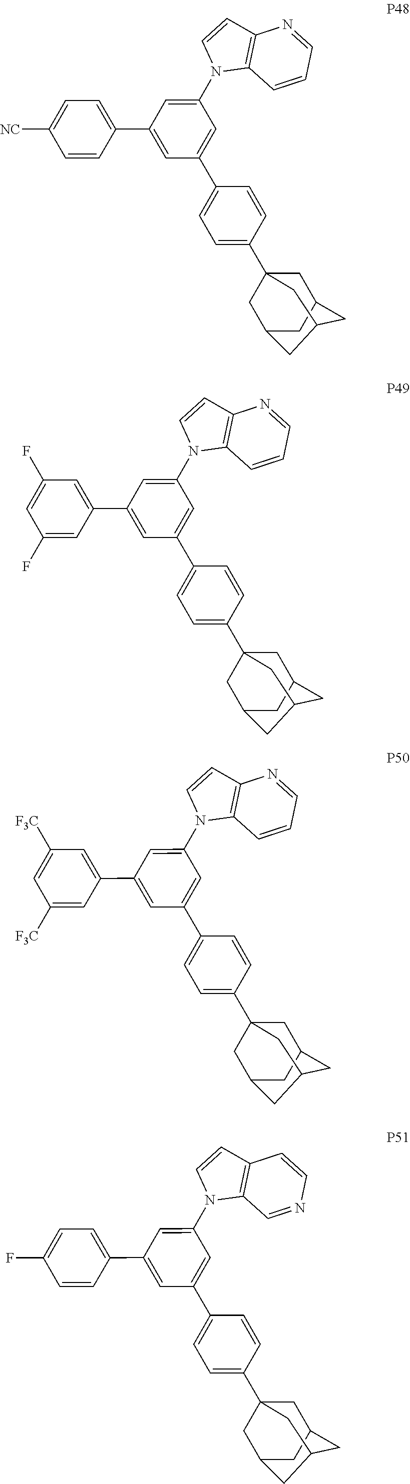

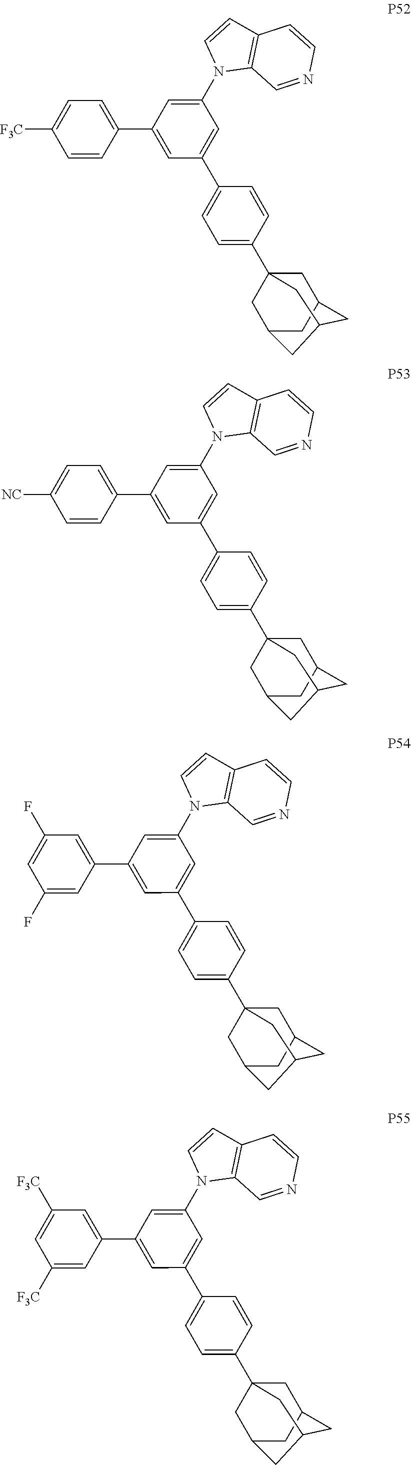

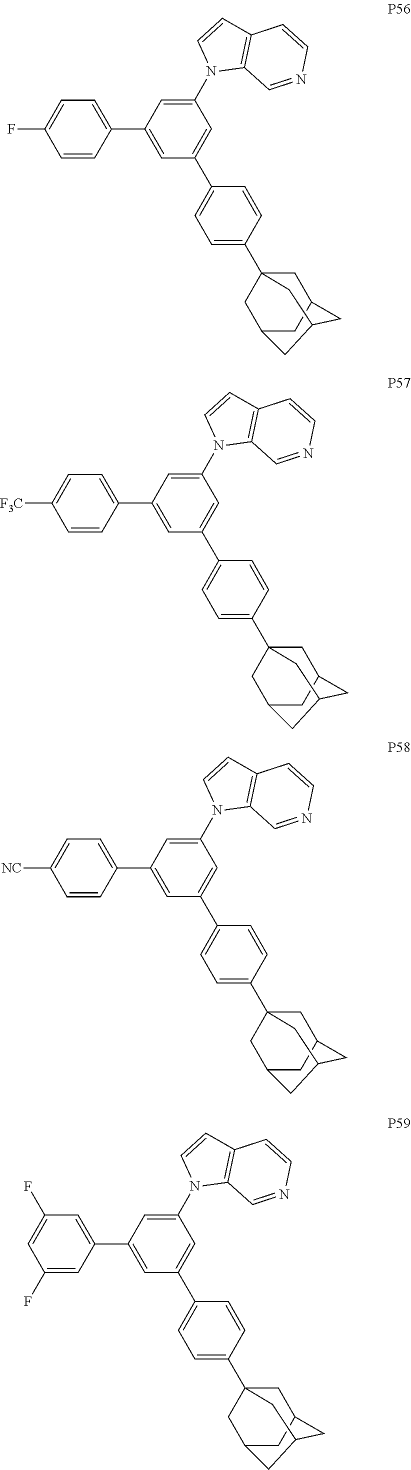

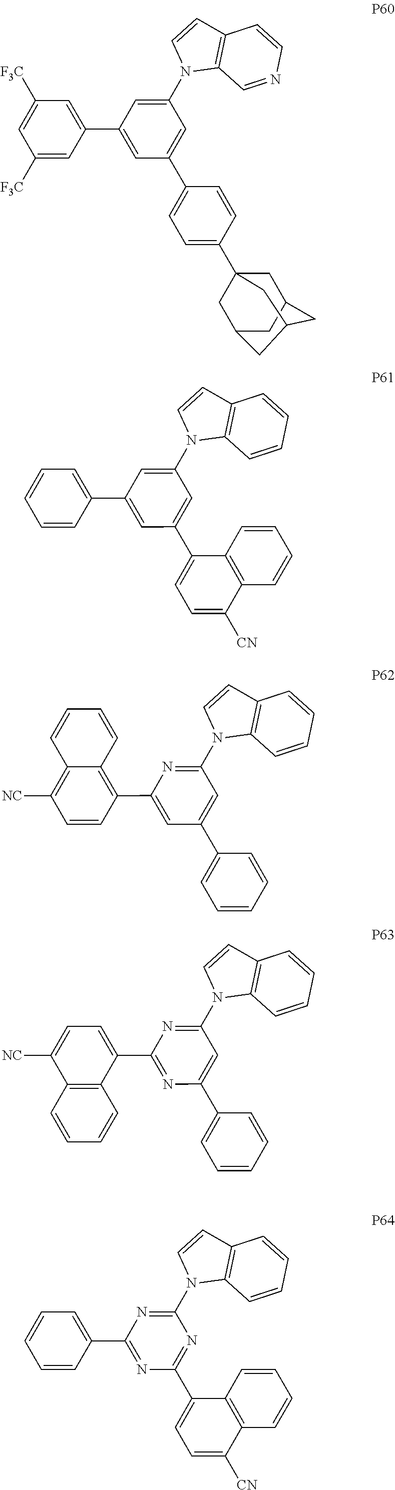

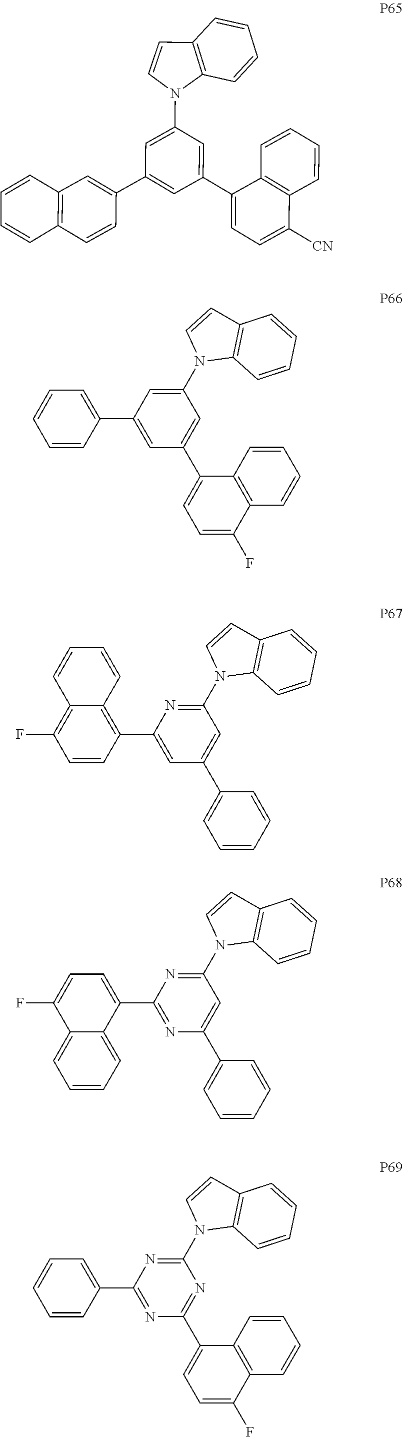

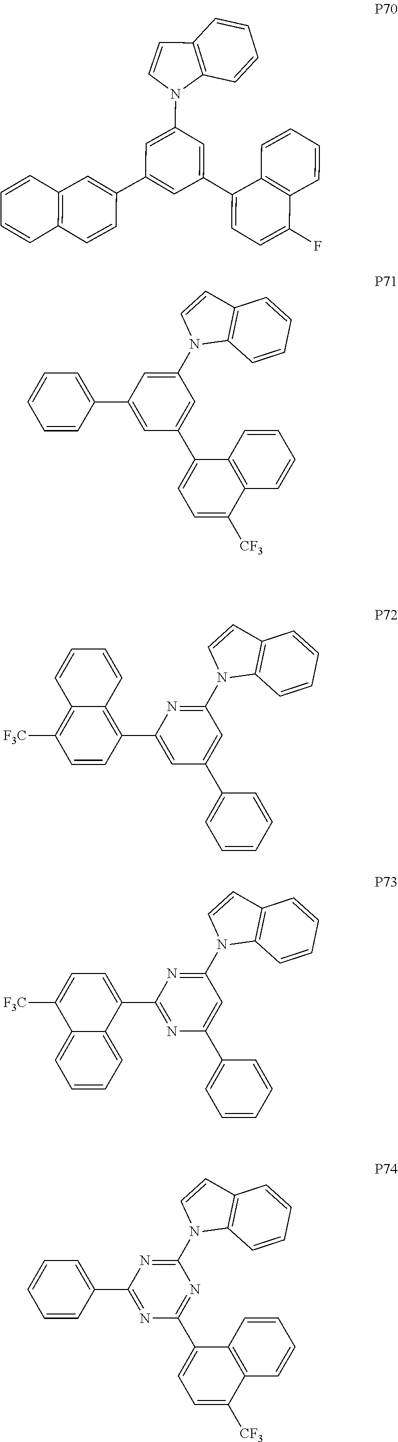

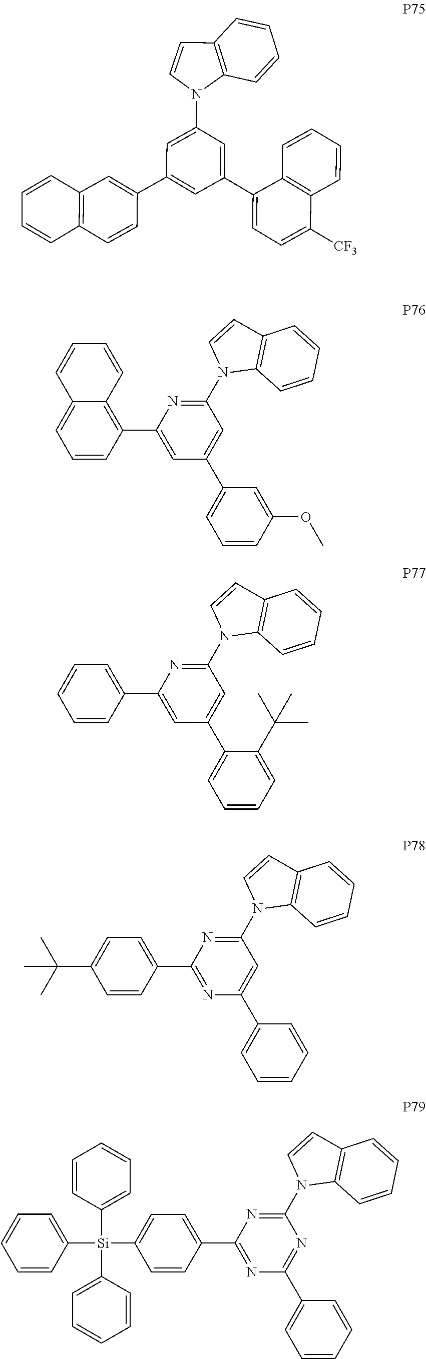

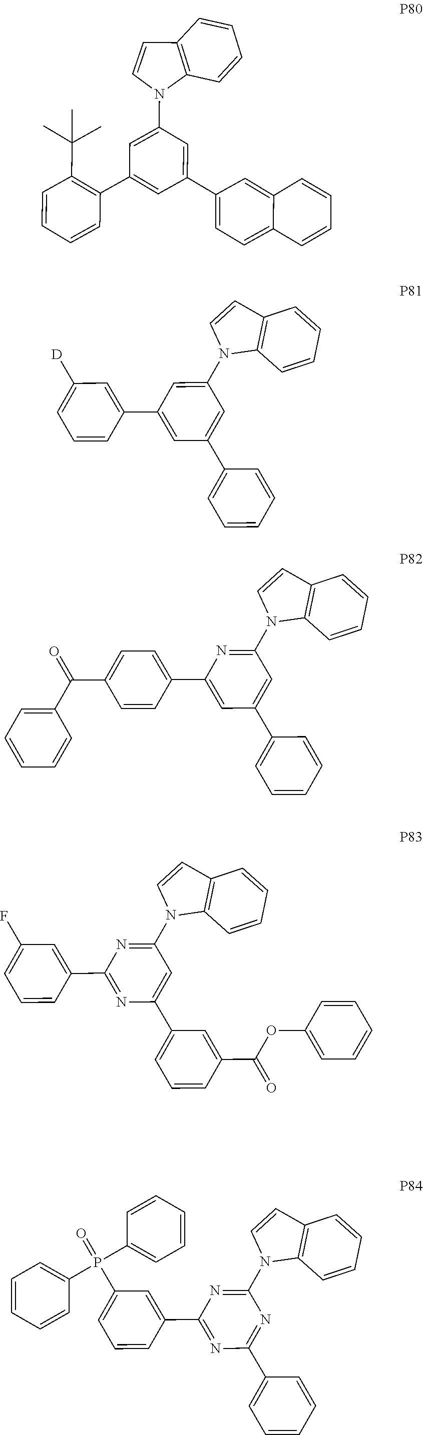 [SMILES: CC(C)(C)c1ccccc1-c1cc(-c2ccc3ccccc3c2)cc(-n2ccc3ccccc32)c1 ; [2H]c1cccc(-c2cc(-c3ccccc3)cc(-n3ccc4ccccc43)c2)c1 ; O=C(c1ccccc1)c1ccc(-c2cc(-c3ccccc3)cc(-n3ccc4ccccc43)n2)cc1 ; O=C(Oc1ccccc1)c1cccc(-c2cc(-n3ccc4ccccc43)nc(-c3cccc(F)c3)n2)c1 ; O=P(c1ccccc1)(c1ccccc1)c1cccc(-c2nc(-c3ccccc3)nc(-n3ccc4ccccc43)n2)c1]

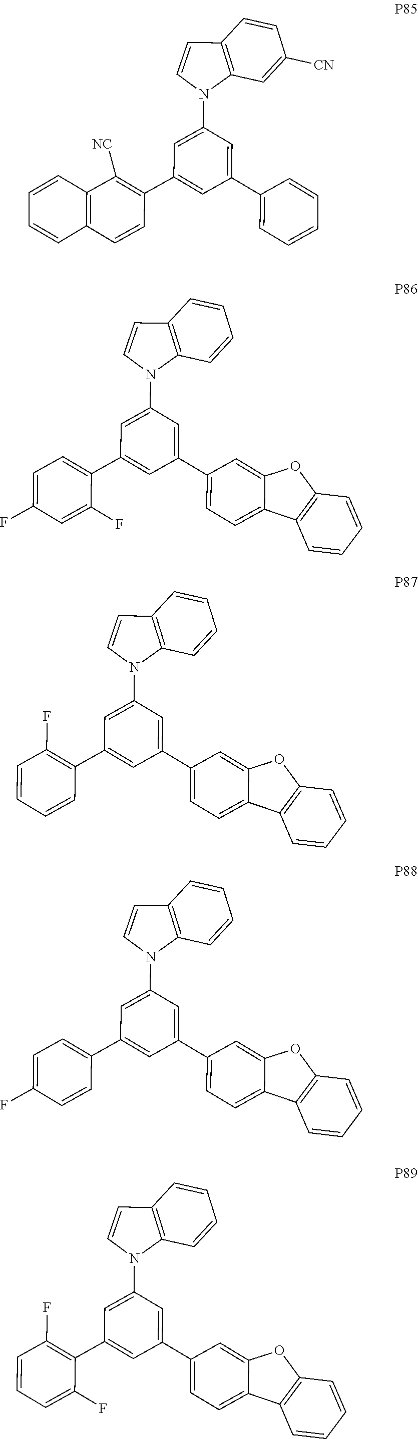 [SMILES: N#Cc1ccc2ccn(-c3cc(-c4ccccc4)cc(-c4ccc5ccccc5c4C#N)c3)c2c1 ; Fc1ccc(-c2cc(-c3ccc4c(c3)oc3ccccc34)cc(-n3ccc4ccccc43)c2)c(F)c1 ; Fc1ccccc1-c1cc(-c2ccc3c(c2)oc2ccccc23)cc(-n2ccc3ccccc32)c1 ; Fc1ccc(-c2cc(-c3ccc4c(c3)oc3ccccc34)cc(-n3ccc4ccccc43)c2)cc1 ; Fc1cccc(F)c1-c1cc(-c2ccc3c(c2)oc2ccccc23)cc(-n2ccc3ccccc32)c1]

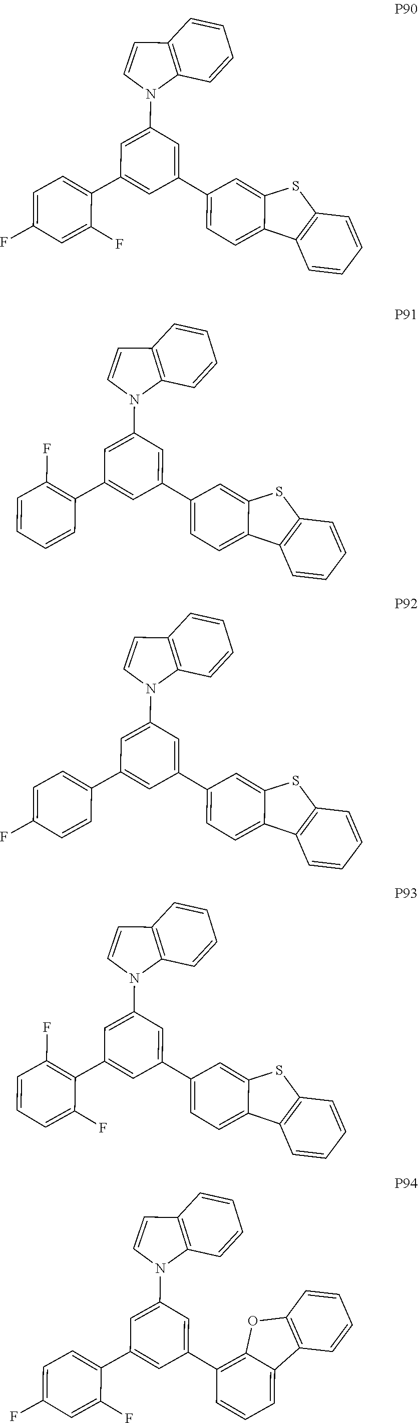 [SMILES: Fc1ccc(-c2cc(-c3ccc4c(c3)sc3ccccc34)cc(-n3ccc4ccccc43)c2)c(F)c1 ; Fc1ccccc1-c1cc(-c2ccc3c(c2)sc2ccccc23)cc(-n2ccc3ccccc32)c1 ; Fc1ccc(-c2cc(-c3ccc4c(c3)sc3ccccc34)cc(-n3ccc4ccccc43)c2)cc1 ; Fc1cccc(F)c1-c1cc(-c2ccc3c(c2)sc2ccccc23)cc(-n2ccc3ccccc32)c1 ; Fc1ccc(-c2cc(-c3cccc4c3oc3ccccc34)cc(-n3ccc4ccccc43)c2)c(F)c1]

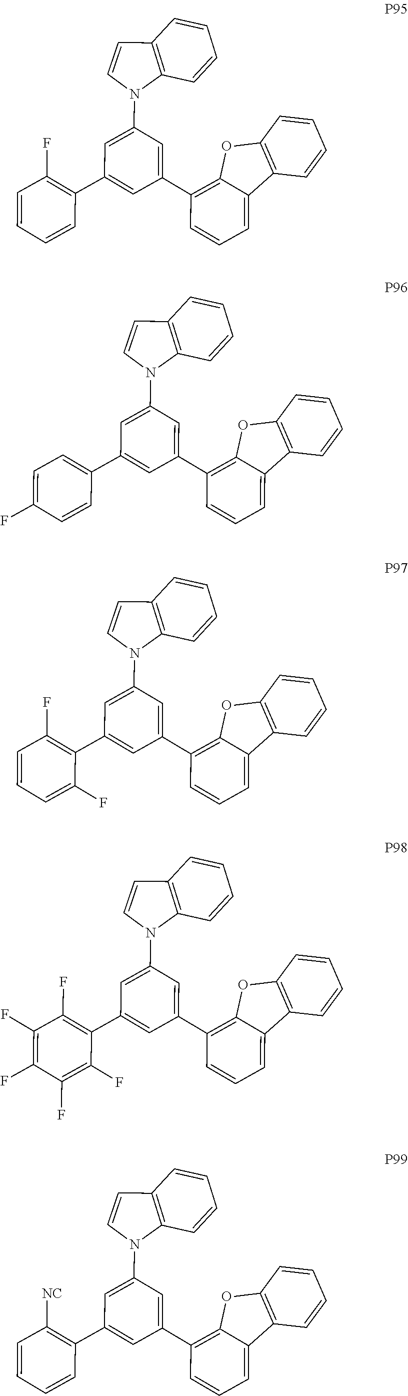

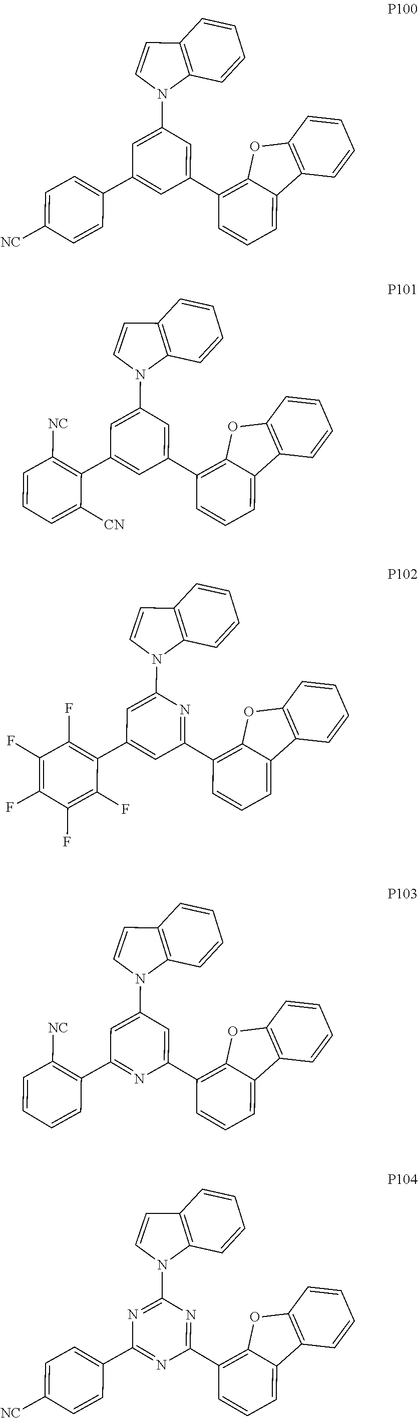

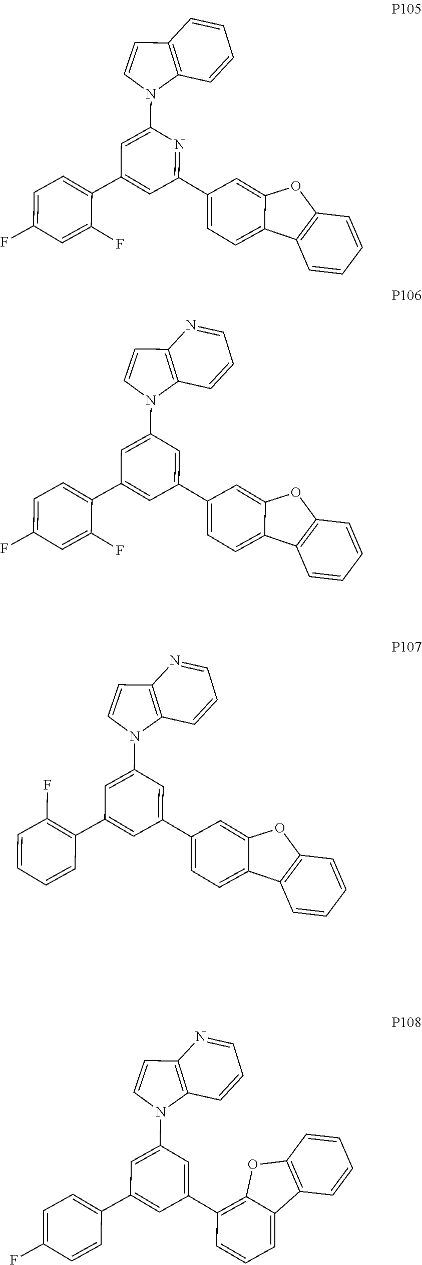 [SMILES: Fc1ccc(-c2cc(-c3ccc4c(c3)oc3ccccc34)nc(-n3ccc4ccccc43)c2)c(F)c1 ; Fc1ccc(-c2cc(-c3ccc4c(c3)oc3ccccc34)cc(-n3ccc4ncccc43)c2)c(F)c1 ; Fc1ccccc1-c1cc(-c2ccc3c(c2)oc2ccccc23)cc(-n2ccc3ncccc32)c1 ; Fc1ccc(-c2cc(-c3cccc4c3oc3ccccc34)cc(-n3ccc4ncccc43)c2)cc1]

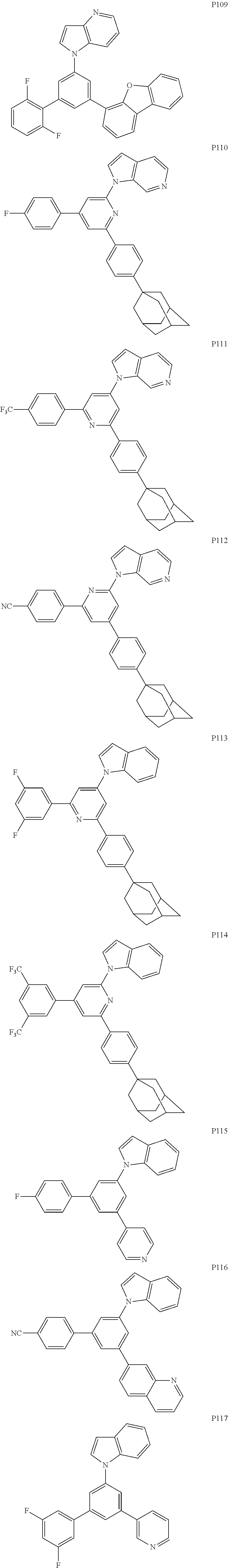

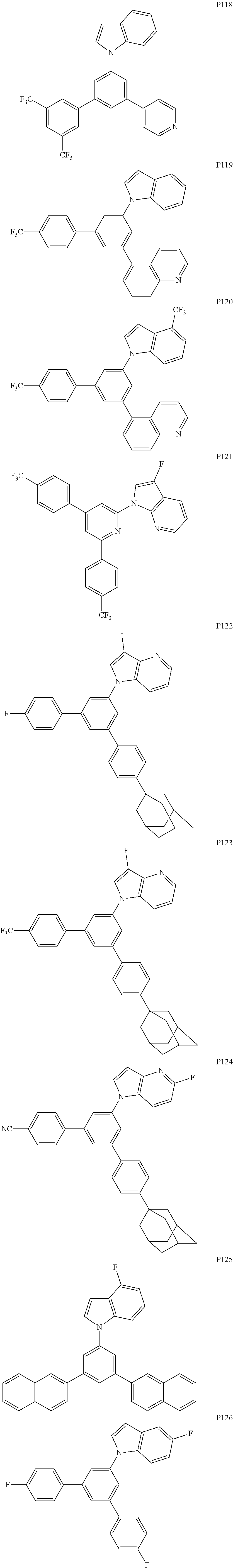

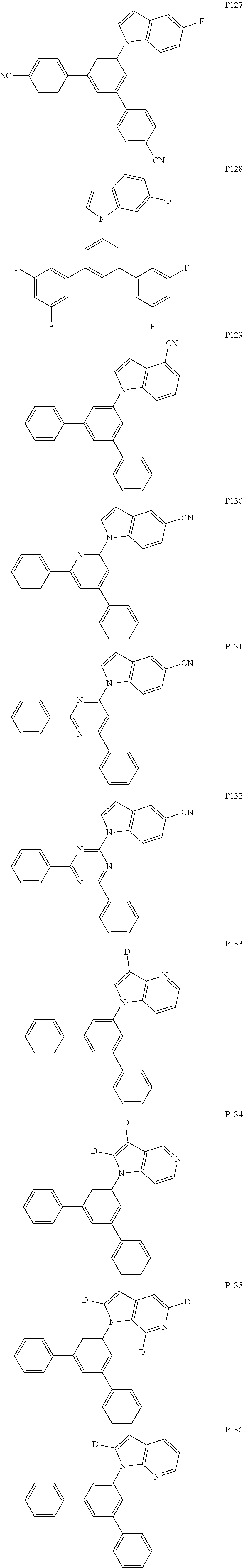

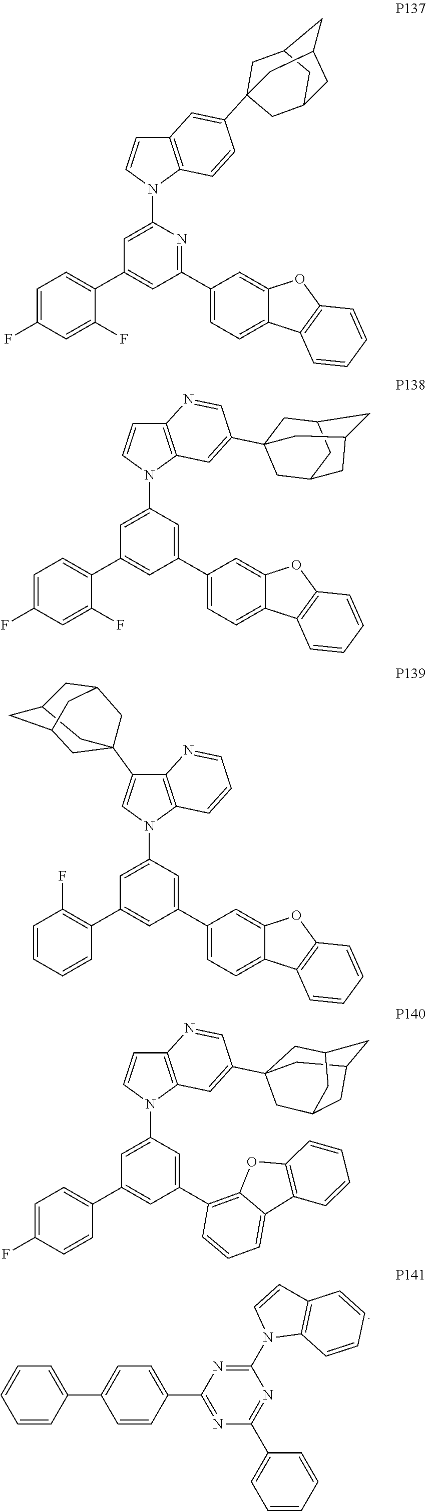

14. An organic electroluminescent element, comprising an anode, a cathode and an organic layer disposed between the anode and the cathode, wherein a first capping layer is further disposed on a side of the cathode facing away from the anode, and the first capping layer comprises the organic compound according to claim 1.

15. The organic electroluminescent element according to claim 14, wherein a second capping layer is further disposed between the first capping layer and the cathode, a refractive index of a material of the second capping layer is larger than a refractive index of the organic compound, and the refractive index of the organic compound in a visible light wavelength region is 1.5 to 1.7.

16. A display device, comprising the organic electroluminescent element according to claim 14.
